(19) United States
(12) Patent Application Publication
(10) Pub. No.: US 2021/0015857 A1
(43) Pub. Date: Jan. 21, 2021

(54) METHOD FOR IMMUNOTHERAPY DRUG TREATMENT

(71) Applicant: The University of Western Australia, Nedlands (AU)

(72) Inventors: Willem Joost Lesterhuis, Swanbourne (AU); Richard Lake, Nedlands (AU); Rachael Zemek, Shenton Park (AU); Anthony Bosco, Perth (AU); Emma De Jong, East Victoria Park (AU)

(21) Appl. No.: 16/979,962

(22) PCT Filed: Mar. 22, 2019
(86) PCT No.: PCT/AU2019/050259

§ 371 Date: Sep. 11, 2020

(30) Foreign Application Priority Data
Mar. 23, 2018 (AU) 2018900962

(51) Int. Cl. A61K 35/13; C07K 16/28; C07K 16/24; C07K 14/57; C12N 1/38

(52) U.S. Cl. CPC A61K 35/13; C07K 16/2878; C12N 1/38; C07K 14/57; C07K 16/244

(57) ABSTRACT

The present invention provides a method that at least promotes, drives or directs a non-responsive neoplastic microenvironment towards a responsive phenotype. More particularly, the invention provides a method for enhancing the sensitivity of one or more neoplastic tumour to check point blockade agents. The present invention also provides a method for predicting response to immune checkpoint blockade.

### TECHNICAL FIELD

The present invention relates to a method that promotes, drives or directs neoplastic cells and/or tumours which are non-responsive to checkpoint blockade agents towards a responsive phenotype. More particularly, the invention provides a method for enhancing the sensitivity of one or more neoplastic cells and/or tumours to checkpoint blockade agents. The invention also provides a method for predicting a response to certain immunotherapy.

### BACKGROUND ART

The following discussion of the background art is intended to facilitate an understanding of the present invention only. The discussion is not an acknowledgement or admission that any of the material referred to herein is or was part of the prior art base or formed part of the common general knowledge as at the priority date of the claims of this application.

Immune checkpoints are pathways which regulate the immune system and play a role in self-tolerance which prevents the immune system from attacking cells indiscriminately. Inhibitory checkpoint molecules are targets for cancer immunotherapy due to their potential for use against multiple types of cancer. For example, currently approved check point inhibitors block CTLA4 and PD-1 and PD-L1. Drugs, drug candidates or other molecules such as monoclonal antibodies that inhibit/block the inhibitory checkpoint molecules are frequently referred to as immune checkpoint inhibitors or, more simply, checkpoint inhibitors. The inhibition of immune checkpoint is referred to as immune checkpoint blockade (ICB), or simply checkpoint blockade.

Cancer immunotherapy using antibodies that target immune check points has shown outstanding results in the past few years (Lesterhuis, W. J., et al., (2011) Nat Rev Drug Discov 10, 591-600). Specifically, antibodies targeting immune checkpoints such as programmed death receptor 1 (PD-1) and cytotoxic T cell associated protein 4 (CTLA-4) can prolong survival in some patients with various cancer types including melanoma, non-small cell lung cancer and several other various cancer types (Hodi, F. S. et al. (2010) New Engl J Med 363, 711-723; Reck, M. et al., (2016) N Engl J Med 375, 1823-1833; and Wolchok, J. D. et al. (2013) N Engl J Med 369, 122-133). However, responses to such immunotherapies occurs only in a subset of patients with a large proportion of patients not responding and not benefiting. For example, some patients have an immediate and complete regression of all their tumors, sometimes within weeks (P. B. Chapman et al., (2015) N Engl J Med 372, 2073-2074), while other patients do not experience any therapeutic response whatsoever (Wolchok, J. D. et al. (2013) N Engl J Med 369, 122-133. Accordingly, although treatment with ICB improves survival in many cancers, many patients do not benefit, and some cancer types seem less sensitive/responsive (The Lancet, O. (2017) Lancet Oncol 18, 981). Furthermore, it is not fully understood what biological processes determine an effective outcome (Lesterhuis, W. J. et al. (2017) Nat Rev Drug Discov 16, 264-272). This lack of understanding hinders the development of rational combination treatments.

Although several correlates of response to immune checkpoint blockade (ICB) have been reported, such as expression of checkpoint ligands (Herbst, R. S. et al. (2014) Nature 515, 563-567), mutational load (M. Yarchoan et al., (2017) N Engl J Med 377, 2500-2501), neoantigen expression (Van Allen, E. M. et al. (2015) Science 350, 207-211), interferon signatures (Ayers, M. et al. (2017) J Clin Invest 127, 2930-2940) and an inflammatory tumour microenvironment (Ji, R. R. et al. (2012) Cancer Immunol Immunother 61, 1019-1031), no definitive predictive biomarkers have been identified (Lesterhuis, W. J. et al. (2017) Nat Rev Drug Discov 16, 264-272).

More importantly, it has been unclear if it is possible to manipulate a non-responsive tumour microenvironment towards a responsive phenotype and, if so, how. Consequently, the clinical development of combination therapies has been largely empiric, focusing on modulating specific cell types or pathways, and sometimes based on scant preclinical data (Farkona, S., et al., (2016) BMC Med 14, 73).

With over 2000 current clinical trials involving ICB worldwide (J. Tang et al., (2018) Nat Rev Drug Discov 17, 854-855), there is a clear need to prioritize immunotherapy combinations, preferably based on preclinical data, so as to limit patient exposure to futile treatments and potentially severe side effects. Ideally, this would involve pharmacologically altering the tumour microenvironment to a favourable phenotype before therapy, followed by subsequent assessment of the tumour state to determine whether or not to proceed with checkpoint-targeted treatment. Such a strategy would prioritize immunotherapeutic combinations to test clinically in patients and would also allow effective patient stratification in experimental trials and clinical practice.

Previous attempts to define a signature predicting response to ICB have not been successful, potentially due to differences in germline genetics, environmental factors and the diverse genetic and cellular make up of cancers (Hugo, W. et al. (2017) Cell 168, 542; and Riaz, N. et al. (2017) Cell 171, 934-949).

Interestingly, even in the highly homogeneous setting of inbred mouse strains bearing tumours derived from monoclonal cancer cell lines, there remains a dichotomy in responsiveness to immunotherapy treatment with immune checkpoint blockade agents (W. J. Lesterhuis et al., (2017) Nat Rev Drug Discov 16, 264-272; W. J. Lesterhuis et al., (2015) Sci Rep 5, 12298; S. Chen et al., (2015) Cancer Immunol Res 3, 149-160; S. R. Woo et al., (2012) Cancer Res 72, 917-927; R. P. Sutmuller et al., (2001) J Exp Med 194, 823-832; M. A. Curran, et al., Proceedings of the National Academy of Sciences of the United States of America (2010)107, 4275-4280; and J. F. Gross, & M. N. Jure-Kunkel, (2013) Cancer immunity 13, 5), see also FIG. 1a. This is remarkable since their genomes are identical and the tumours are derived from a clonal cell line, thus excluding differences in self and mutated tumour rejection antigens. In these experiments, the mice were of the same age and gender, were kept under controlled, pathogen-free conditions, and receive identical treatment.

It is against this uncertain background that the present invention has been developed.

### SUMMARY OF INVENTION

In the work leading to the present invention, the inventors sought to identify a signature in the microenvironment of a population of neoplastic cells such as tumours before treatment with ICB agents that would correlate with response to immunotherapy with ICB agents.

The inventors also sought to develop a method that would turn non-responsive neoplastic cellular microenvironment towards a responsive phenotype for certain immunotherapeutic agents. In particular, the inventors sought to develop a method for promoting or enhancing the sensitivity of neoplastic cell population and/or tumours to one or more immune checkpoint blockade agents.

The inventors also sought to provide a therapeutic composition comprising one or more sensitising agents that can promote or enhance sensitivity of neoplastic cell population and/or tumours to one or more immune checkpoint blockade agents.

They also sought to develop a diagnostic method for predicting a response to immunotherapy of neoplastic cell population and/or tumours to immunotherapy with one or more immune checkpoint blockade agents.

Accordingly, the present invention provides a principal of very general application that seeks to drive an immunotherapeutic non-responsive neoplastic cellular microenvironment towards a responsive phenotype for certain immunotherapeutic agents. Using this method neoplastic cell populations and/or neoplastic tumours can be sensitised, changing their phenotype, to make them susceptible or more susceptible to immune checkpoint blockade agents. Furthermore, using this method effector immune cells such as interferon gamma (IFNγ) and/or activated signal transducer and activator of transcription 1 (STAT1) protein producing natural killer cells (NK) are targeted and become immobilised and infiltrate the non-responsive neoplastic cellular microenvironment (such as at tumour site) to promote or enhance sensitivity of neoplastic cells and/or tumours to ICB agents. In addition, using this method immune effectors such as activated STAT1 and IFNγ are increased in the neoplastic cellular microenvironment (such as at tumour site) to promote or enhance sensitivity of non-responsive neoplastic cells and/or tumours to ICB agents. In particular, the invention provides for one or more sensitising agents that can make immunotherapy-resistant neoplastic cells and/or tumours, sensitive. In one example, this particularly relates to treatment with checkpoint-blocking antibodies.

In addition, or in the alternative, the invention delivers a method for predicting a response to immune checkpoint blockade. Particularly, the inventors have identified a method to assess whether the method of the invention has successfully sensitized neoplastic cells prior to immunotherapy.

In one aspect, the invention resides in a method for promoting or enhancing the sensitivity of one or more neoplastic cells and/or neoplastic tumours to immune checkpoint blockade agents, said method comprising the step of: administering to a neoplastic cell and/or a neoplastic tumour, prior to treatment of immune checkpoint blockade agents, one or more immune checkpoint sensitising agents or exposing the cell and/or tumour to the one or more sensitising agents to thereby cause the cells and/or tumour to become sensitized to an immune checkpoint blockade agent.

In a related embodiment of this aspect, there is provided a method for promoting or enhancing the sensitivity of one or more neoplastic cells and/or neoplastic tumours to immune checkpoint blockade agents, said method comprising the step of:
a. administering to a neoplastic cell and/or a neoplastic tumour, prior to treatment of immune checkpoint blockade agents, one or more immune checkpoint sensitising agents, or exposing the cell and/or tumour to the one or more sensitising agents, for a period of time and/or at a therapeutic amount that causes a tumour to become sensitized to an immune checkpoint blockade agent.

In one example, the immune checkpoint sensitising agents are selected from: a CD40 agonist, an anti-IL10 antibody, an inducer of interferon alpha/beta signalling, an interferon gamma or a functional variant thereof and/or a retinoid.

In one such example, the CD40 agonist may be an agonistic CD40 antibody or a CD40 ligand. In one preferred example, the CD40 agonist is an agonistic CD40 antibody.

In another example, the inducer of interferon alpha/beta signalling is a toll-like receptor 3 (TLR3) ligand. For example, the inducer of interferon alpha/beta signalling may be a TLR3 ligand selected from the group consisting of: poly(I:C), poly(A:U), poly ICLC, polyl:polyC12U, and sODN-dsRNA. Preferably, the inducer of interferon alpha/beta signalling is or comprises poly(I:C).

In another example, the retinoid is selected from tretinoin, retinol, retinal, isotretinoin, alitretinoin, etretinate, acitretin, adapalene, bexarotene, and/or tazarotene. Preferably, the retinoid is tretinoin and/or bexarotene and/or isotretinoin. More preferably, the retinoid is tretinoin.

Preferably, the immune checkpoint sensitising agents are selected from the group comprising: an agonistic CD40 antibody, anti-IL10, Poly(I:C), a retinoid and/or Interferon gamma.

Preferably, the sensitising agents are administered to the neoplastic cell and/or tumour for sufficient time, prior to the introduction of the immune checkpoint blockade agent, to sensitize the cell and/or tumour to the immune checkpoint blockade agent(s). Alternatively, or in addition, the neoplastic cell and/or tumour is exposed to the sensitising agents for sufficient time, prior to the introduction of the immune checkpoint blockade agent, to sensitize the cell and/or tumour to the immune checkpoint blockade agent(s). In one exemplary form of the invention, the sensitising agent is brought in contact with a neoplastic cell and/or tumour that is non-responsive to immune checkpoint agents for at least 3 days prior to immunotherapy. More particularly, the sensitising therapeutic is made to contact with the tumour for between 3 days and 5 weeks at a clinical standard non-toxic dose. In an alternate form of the invention, the sensitising agent is brought in contact with a neoplastic cell and/or tumour that is non-responsive to immune checkpoint agents for such time to activate signal transducer and activator of transcription 1 (STAT1) protein.

Preferably, the sensitising agents are administered or contacted with the cell or tumour at a concentration or effective dose that is sufficient to cause a neoplastic cell and/or tumour to be sensitized prior to administration of the immune checkpoint blockade agents. Notably, the amounts of the agent(s) effective for this purpose will vary depending on the type of agent used, as well as the particular factors of each case, including the type of condition, the stage of the condition, the subject's weight, the severity of the subject's condition, and the method of administration. Ideally the concentration of sensitising agent used in the method will be sufficient to activate STAT1 protein in a tumour cell population.

In one embodiment of the first aspect of the invention there is provided a method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase the numbers of NK cells and thereby promote or enhance the sensitivity of the neoplastic cell and/or tumour to an immune check point blockage agent.

Preferably, the NK cells according to any broad aspect, embodiment, form or example of the invention described herein throughout are NK cells which produce IFNγ and/or activated STAT1 protein.

The activated STAT1 protein according to any broad aspect, embodiment, form or example of the invention described herein throughout is typically a phosphorylated STAT1 protein.

In one preferred example, the method causes an increase in the numbers of NK cells at the site of the neoplastic cell and/or tumour and/or at the cellular microenvironment of the neoplastic cell and/or tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In another embodiment of the first aspect of the invention there is provided a method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by the cell and/or tumour thereby promoting or enhancing the sensitivity of the one or more neoplastic cells an immune check point blockage agent.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In yet another embodiment of this first aspect of the invention, there is provided a method for promoting or enhancing the sensitivity of a neoplastic cell and/or neoplastic tumour to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by NK cells thereby promoting or enhancing the sensitivity of the neoplastic cell and/or tumour to an immune check point blockage agent.

In one preferred example, the increased production of IFNγ and/or activated STAT1 protein by NK cells occurs at the site of the neoplastic cell and/or tumour and/or in the microenvironment of the neoplastic cell and/or tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In another embodiment of the first aspect of the invention there is provided a method for promoting or enhancing the sensitivity of a neoplastic cell and/or tumour to immune checkpoint blockade agents, said method comprising the step of:
a. identifying a neoplastic cell and/or tumour that is resistant to one or more immune checkpoint blockade agents; and
b. administering or exposing the neoplastic cell and/or tumour identified in step (a) to a therapeutically effective amount of one or more immune checkpoint sensitising agents, for at least 3 days prior to immunotherapy or until the tumour is at least partially sensitized to an immune checkpoint blockade agent.

In one example, the neoplastic cell is a neoplastic cell in a tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

More preferably, the immune checkpoint sensitising agents are selected from the group comprising: an agonistic CD40 antibody, anti-IL10, Poly(I:C), a retinoid (such as all-trans retinoic acid) and/or Interferon gamma.

In a preferred form of the method, a combination of immune checkpoint sensitising agents are used in the method, said combination being at least a plurality of the identified sensitising agents selected from the group comprising: a CD40 agonist, an anti-IL10 antibody, an inducer of interferon alpha/beta signalling, and an interferon gamma or a functional variant thereof.

Preferably, the immune checkpoint sensitising agents comprise at least a retinoid, for example tretinoin. In one example, the immune checkpoint sensitising agents comprise at least a retinoid and any one or more of a CD40 agonist and/or an anti-IL10 antibody and/or an inducer of interferon alpha/beta signalling and/or an interferon gamma or a functional variant thereof.

Preferably, the immune checkpoint sensitising agents comprise at least an inducer of interferon alpha/beta signalling such as Poly(I:C). In one example, the immune checkpoint sensitising agents comprise an inducer of interferon alpha/beta signalling such as Poly(I:C), an anti-IL10 antibody and interferon gamma or a functional variant thereof. In another example, the immune checkpoint sensitising agents comprise at least an inducer of interferon alpha/beta signalling such as Poly(I:C), and any one or more of: anti-IL10 antibody and/or interferon gamma or a functional variant thereof and/or a CD40 agonist such as agonistic CD40 antibody. In one such example, the immune checkpoint sensitising agents comprise at least an inducer of interferon alpha/beta signalling such as Poly(I:C), and any one or both of an anti-IL10 antibody and/or interferon gamma or a functional variant thereof. In another example, the immune checkpoint sensitising agents comprise at least a CD40 agonist such as an agonistic anti-CD40 antibody.

Preferably, said combination being at least a plurality of the identified sensitising agents selected from the group comprising: an agonistic CD40 antibody, anti-IL10, Poly(I:C), a retinoid (such as all-trans retinoic acid) and/or Interferon gamma. Ideally, the combination will be a combination of at least three of anti-IL10, Poly(I:C) and interferon gamma or anti-CD40, anti-IL10, a retinoid such as all-trans retinoic acid combination of a STAT1-activating cytokine IFNγ. For example, the combination can be a STAT1-activating cytokine IFNγ, the TLR3 ligand poly(I:C) and an anti-IL-10 antibody.

A tumour will be partially sensitized to an immune check point blockade agent when there is at least a 5% response of the neoplastic cells in the tumour to immune checkpoint blockade with an immune checkpoint blockade agent.

Preferably, the neoplastic cell population in step (a) of this method is selected by either (i) exposing the cells to one or more immune checkpoint blockade agents and identifying those cells that are resistant to the immune checkpoint blockade agents or (ii) by measuring the activity of STAT1 in a cell population, which cell population may be of tumour or immune origin, wherein the absence of activation of the STAT1 protein (which may be measured by either nuclear STAT1 or phosphorylated STAT1 in a cell population, with a threshold of 50%) presents as biomarker for resistance of that cell population in step (a) to immune checkpoint blockade agents. In one example, a threshold of 50% measure for nuclear STAT1 presents a biomarker for resistance for that cell population in step (a) to immune checkpoint blockade agents. In another example, a threshold of 5% measured for phosphorylated STAT1 presents a biomarker for resistance for that cell population in step (a) to immune checkpoint blockade agents.

In another form of this method, the cells of step (b) will have been exposed to an immune checkpoint blockade agent for a sufficient period of time when measurable amounts of the STAT1 biomarker is detected in the cell population. Such measurable amounts are preferably at least a 40% response, but more preferably at least 50% response in a nuclear STAT1 test and/or at least 5% response in phosphorylated STAT1 test, as herein described.

In this preferred form of the invention, the cell population in step (b) is measured on a periodic basis (optionally, hourly or every 2, 6, 12 or 24 hours) for activation of STAT1, wherein the activation and/or presence of the biomarker STAT1 is indicative of cell sensitivity to one or more immune checkpoint blockade agents.

In another embodiment of the first aspect of the invention there is provided a use of one or more immune checkpoint sensitising agents, for promoting or enhancing the sensitivity of a tumour to immune checkpoint blockade agents wherein the sensitising agent(s) is administered at a therapeutically effective amount at least 3 days prior to immunotherapy to a tumour that is resistant to one or more immune checkpoint blockade agents.

In a preferred form of the invention the method also includes the step of administering an immunotherapy or an immune checkpoint blockade agent once the one or more checkpoint sensitising agents have attracted sufficient effector immune cells (e.g. IFNγ and/or activated STAT1 producing NK cells) to the tumour or neoplastic cell population environment for enhancing the efficacy of the immune therapy or immune checkpoint blockade agent(s) on a malignant condition.

In another preferred form, the method includes the step of administering an immunotherapy or an immune checkpoint blockade agent once the one or more checkpoint sensitising agents have resulted in sufficient increase in the amount of the immune effectors IFNγ and/or activated STAT1 at the tumour or neoplastic cell population environment for enhancing the efficacy of the immune therapy or immune checkpoint blockade agent(s) on a malignant condition. For example, the IFNγ and/or activated STAT1 is produced by NK cells and/or by the tumour or neoplastic cell population.

According to the invention, the immune checkpoint blockade agent(s) that is selected for use in the method is an agent that targets the inhibitory T cell molecule CTLA-4 and/or targets the Programmed Death receptor (PD-1) and/or or PD-Ligand (PD-L) pathway and/or and/or glucocorticoid-induced tumour necrosis factor receptor (GITR) and/or Lymphocyte-activation gene (LAG)3 tumor necrosis factor receptor superfamily, member 4, also known as CD134 and/or OX40 and/or 41BB and/or t-cell immunoglobulin and mucin-domain containing-(TIM)3. An example of an agent that targets the inhibitory T cell molecule CTLA-4 is a CTLA-4 antagonist such as ipilimumab or tremelimumab. An example of an agent that targets PD-1 is a PD-1 antagonist such as nivolumab, AMP-224, pidilizumab, spartalizumab, cemiplimab, camrelizumab, tislelizumab or pembrolizumab. An example of an agent that targets PD-L1 is a PD-L1 antagonist such as Atezolizumab, Avelumab or Durvalumab. Examples of agents that target GITR are antagonists such as TRX518 or MK4166. Examples of agents that target LAG3 are BMS-986016, BI 754111, LAG-525 or REGN-3767. An example of an agent that targets OX40 is BMS 986178, MED16469, GSK3174998, PF-04518600. Examples of agents that target TIM3 are LY3321367, MBG453 or TSR-022. An example of an agent that targets 41BB is PF-05082566.

According to a second aspect, the invention resides in the use of a therapeutically effective amount of one or more immune checkpoint sensitising agents, in the manufacture of a medicament for sensitising a tumour wherein said tumour is resistant to an immune checkpoint blockade agent.

In a related embodiment the invention resides in the use of a therapeutically effective amount of one or more immune checkpoint sensitising agents, in the manufacture of a medicament for sensitising a tumour wherein said tumour is resistant to an immune checkpoint blockade agent, wherein said medicament increases the numbers of NK cells (such as NK cells producing activated STAT1- and/or IFNγ) and/or increases IFNγ and/or activated STAT1 production by neoplastic cells and/or tumour cells and/or NK cells.

Preferably, the medicament includes instructions to administered to a tumour that is resistant to one or more immune checkpoint blockade agents the immune checkpoint sensitising agents at least 3 days prior to an immunotherapy.

Preferably, the immune checkpoint sensitising agents are selected from: a CD40 agonist, an anti-IL10 antibody, an inducer of interferon alpha/beta signalling, an interferon gamma or a functional variant thereof and/or a retinoid. More preferably, the immune checkpoint sensitising agents are selected from the group comprising: an agonistic CD40 antibody, anti-IL10, Poly(I:C), a retinoid (such as all-trans retinoic acid) and/or Interferon gamma. In a particularly preferred form, a combination of immune checkpoint sensitising agents are used in the method, said combination being at least a plurality of the identified sensitising agents selected from the group comprising: an agonistic CD40 antibody, anti-IL10, Poly(I:C), a retinoid (such as all-trans retinoic acid) and/or Interferon gamma. Ideally, the combination will be a combination of at least three of an anti-IL10, Poly(I:C) and interferon gamma or anti-CD40, anti-IL10, a retinoid such as all-trans retinoic acid or a STAT1-activating cytokine IFNγ. For example, the combination can be a STAT1-activating cytokine IFNγ, the TLR3 ligand poly(I:C) and an anti-IL-10 antibody.

According to a third aspect, the invention resides in a method for treating a patient with either (1) a malignant condition or (2) a post-operative surgical resection of cancer or (3) in advance of, during or following any other form of adjuvant immunotherapy, said method comprising the step of:
a. identifying a tumour or neoplastic cell population(s) that is resistant to one or more immune checkpoint blockade agents; and
b. administering or exposing the tumour or neoplastic cell population(s) identified in step (a) to a therapeutically effective amount of one or more immune checkpoint sensitising agents, for at least 3 days prior to immunotherapy until the tumour is at least partially sensitized to an immune checkpoint blockade agent.

In a preferred form of the third aspect of the invention the method also includes the step of administering an immunotherapy or an immune checkpoint blockade agent once the one or more checkpoint sensitising agents have attracted sufficient effector immune cells (e.g. IFNγ producing and/or STAT1 expressing NK cells) to the tumour or neoplastic cell population environment to enhancing the efficacy of the immune therapy or immune checkpoint blockade agent(s) on a malignant condition.

Preferably, the tumour or neoplastic cell population(s) in step (a) of this method is selected by either (i) exposing the cells to one or more immune checkpoint blockade agents and identifying those cells that are resistant to the immune checkpoint blockade agents or (ii) by measuring the activity of STAT1 in a cell population which cell population may be of tumour or immune origin, wherein the absence of activation of the STAT1 protein presents as a biomarker for resistance of that cell population in step (a) to immune checkpoint blockade agents.

In another preferred form of this method, said method includes the additional step of exposing the cells of step (b) to an immune checkpoint blockade agent when measurable amounts of (i) the activated STAT1 and/or IFNγ are detected in the cell population and/or (ii) measurable amount of natural killer cells (such as NK cells containing activated STAT1 and/or IFNγ) are detected in the tumour or cell population cellular microenvironment.

In an embodiment of the third aspect of the invention there is provided a use of one or more immune checkpoint sensitising agents, for promoting or enhancing, in a patient, the sensitivity of a tumour to immune checkpoint blockade agents wherein the sensitising agent(s) is/are administered to the tumour that is resistant to one or more immune checkpoint blockade agents at least 3 days prior to immunotherapy. In one example, the one of more sensitising agent(s) may then be further administered concurrently with the administration of the one or more immune checkpoint blockade agents. For example, the one of more sensitising agent(s) may then be further administered concurrently with the administration of the one or more immune checkpoint blockade agents for the duration of the ICB therapy, for example up to at least 3 months, or at least 6, or at least 9 months, or at least 10 months, or at least 11 months, or at least 12 months or at least 13 months or at least 14 months or at least 15 months or at least 16 months or at least 17 months or at least 18 months or at least 19 months or at least 20 months or at least 21 months or at least 22 months or at least 23 months or at least 24 months or more than two years.

According to a fourth aspect, the invention resides in a method of treating a patient with a tumour or neoplastic cell population comprising the step of: treating the tumour or neoplastic cell population with combination of a therapeutically effective amount of a STAT1-activating cytokine IFNγ, a TLR3 ligand poly(I:C) and an anti-IL-10 antibody for sufficient time prior to immunotherapy to attract immune cells and in particular IFNγ producing NK cells into the tumour, sensitizing the tumors to immune checkpoint blockade. In a form of this aspect of the invention, the combination is brought in contact with a tumour for at least 3 days prior to immunotherapy. More particularly, the combination is made to contact with the tumour for between 3 days and 5 weeks at a clinical standard non-toxic dose prior to immunotherapy.

According to a fifth aspect, the invention resides in a sensitising therapeutic comprising at least one immune checkpoint sensitising agent(s), for enhancing the efficacy of immune checkpoint blockade agents on a malignant condition. Preferably, the sensitising composition is a combination of at least a plurality of the identified agents. , Preferably, the combination will be a combination of at least two or at least three of a CD40 agonist, an anti-IL10 antibody, an inducer of interferon alpha/beta signalling, an interferon gamma or a functional variant thereof and/or a retinoid. Ideally, the combination will be a combination of at least three of anti-IL10 antibody, Poly(I:C) and interferon gamma or anti-CD40, anti-IL10, or a retinoid such as all-trans retinoic acid and interferon gamma. For example, the combination can be a STAT1-activating cytokine IFNγ, the TLR3 ligand poly(I:C) and an anti-IL-10 antibody. With each composition there may be included an immunotherapeutic agent that can be administered after the sensitising therapeutic composition once the effect of the sensitising therapeutic composition has had effect.

According to the invention there is also provided a sensitising therapeutic comprising:
a) a therapeutically effective amount of one or more immune checkpoint sensitising agents, and
b) a pharmaceutically acceptable carrier.

The sensitising therapeutic according to the invention can comprise one or more of a CD40 agonist, an anti-IL10 antibody, an inducer of interferon alpha/beta signalling, an interferon gamma or a functional variant thereof and/or a retinoid. In one example, the sensitising therapeutic can comprise an agonistic CD40 antibody, anti-IL10, TLR3 ligand Poly(I:C), a retinoid such as all-trans retinoic acid and/or Interferon gamma. For example, the sensitising therapeutic may be provided as a monotherapy. Preferably, the sensitising therapeutic is provided as a combination of therapeutics which together work to exert their biological effect of sensitizing tumour cells.

Preferably, the administration of the sensitising agents in the therapeutic combination occurs concurrently, sequentially, or alternately. In one example, concurrent administration refers to administration of the sensitising agent and the immune checkpoint blockade agent at essentially the same time. For concurrent co-administration, the courses of treatment may also be run simultaneously. For example, a single, combined formulation of the agents may be administered to the patient.

In one example, the administration of the sensitising agents in the therapeutic combination occurs concurrently with the administration of the one or more immune checkpoint blockade agents. For example, the one of more sensitising agent(s) may be administered concurrently with the administration of the one or more immune checkpoint blockade agents for the duration of the ICB therapy, for example up to at least 3 months, or at least 6, or at least 9 months, or at least 10 months, or at least 11 months, or at least 12 months or at least 13 months or at least 14 months or at least 15 months or at least 16 months or at least 17 months or at least 18 months or at least 19 months or at least 20 months or at least 21 months or at least 22 months or at least 23 months or at least 24 months or more than two years.

According to a sixth aspect, the invention resides in a kit for treating a tumour or a population of neoplastic cells the kit comprising:
a) a therapeutically effective amount of one or more immune checkpoint sensitising agents, and
b) instructions to administer the immune checkpoint sensitising agents to a tumour that is resistant to one or more immune checkpoint blockade agents at least 3 days prior to an immunotherapy.

Preferably, the kit also includes one or more immune checkpoint blockade agents and/or immunotherapeutic agents, and instructions to administer the agent or agents to the tumour or neoplastic cell population once the one or more checkpoint sensitising agents have attracted sufficient effector immune cells (e.g. IFNγ and/or activated STAT1 producing NK cells) to the tumour or neoplastic cell population environment. The effect of this is to cause tumour cell sensitisation, enhancing the efficacy of immune checkpoint blockade agents on a malignant condition.

According to a seventh aspect, the invention resides a diagnostic method for predicting a response to immune checkpoint blockade comprising the steps of:
a. measuring STAT1 activation in a cell population; and
b. determining whether the tumour is resistant to immune checkpoint blockade agents wherein the activation and/or presence of the biomarker STAT1 is indicative of the tumour cells developing sensitivity to one or more immune checkpoint blockade agents.

According to an eighth aspect, the invention resides a diagnostic method for predicting a response to immune checkpoint blockade comprising the steps of:
a. measuring natural killer cell activation and/or abundance in a tumour cell population; and
b. determining whether the tumour is resistant to immune checkpoint blockade agents wherein the activation and/or presence of natural killer cells is indicative of the cells developing sensitivity to one or more immune checkpoint blockade agents.

According to a further aspect, the invention resides a diagnostic method for predicting a response to immune checkpoint blockade comprising the steps of:
a. measuring STAT1 activation and/or IFNγ production in a cell population; and
b. determining whether the tumour is resistant to immune checkpoint blockade agents wherein the activation and/or presence of the biomarker STAT1 is indicative of the tumour cells developing sensitivity to one or more immune checkpoint blockade agents.

Preferably, measuring STAT1 activation and/or IFNγ production comprises measuring STAT1 activation and/or IFNγ and/or a neoplastic cell population and/or a tumour.

According to a ninth aspect of the invention, there is provided a method for immobilising NK cells or increasing the number of NK cells at site of a neoplastic cell and/or tumour in a subject and/or to the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

Preferably, the NK cells produce IFNγ and/or activated STAT1 protein.

According to a tenth aspect of invention, there is provided a method of inducing or increasing production of IFNγ and/or activated STAT1 protein by NK cells in a subject, for example at a site of the neoplastic cell and/or tumour in the subject and/or in the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

In an eleventh aspect of the invention, there is provided a method of inducing or increasing production of IFNγ and/or activated STAT1 protein by a neoplastic cell and/or tumour in a subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

In one example, the method comprises administering to the one or more sensitising agents to a neoplastic cell and/or tumour in a subject or exposing a neoplastic cell and/or tumour in the subject to the one or more sensitising agents prior to treatment with the one or more immune checkpoint blockade agents.

In one example according to any broad aspect, embodiment, form or example of the invention described herein the immune checkpoint sensitising agents are selected from: a CD40 agonist, an anti-IL10 antibody, an inducer of interferon alpha/beta signalling, an interferon gamma or a functional variant thereof and/or a retinoid.

In one example, the neoplastic tumour according to any aspect, embodiment, form or example of the invention as described herein throughout is a malignant or tumour or benign tumour.

In one example, the neoplastic cell or cell population according to any aspect, embodiment, form or example of the invention as described herein throughout is malignant or benign.

In one preferred example, the neoplastic cell or cell population comprise one or more cancer tumour cells selected from melanoma tumours, non-small cell lung cancer tumours, Merkel-cell carcinoma tumours, microsatellite instable colorectal cancer tumours, renal cancer tumours, and/or mesothelioma cancer tumours.

Reference to Colour Figures

This application contains at least one illustration executed in colour. Copies of this patent application publication with colour illustrations will be provided by the Office upon request and payment of the necessary fee.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the following accompanying drawings.

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the following accompanying drawings.

FIG. 1 shows inbred mouse strains, carrying tumours derived from monoclonal cell lines displaying a symmetrical, yet dichotomous response to immune checkpoint blockade (ICB), associated with distinctive gene signature prior to treatment with ICB. Panel A, is a graphical representation showing a representative tumour growth curve of BALB/c mice inoculated with a Renca kidney cancer cell line, treated with anti-CTLA4/anti-PD-L1; pooled data from 3 independent experiments; and showing ICB non-responders (red lines); intermediate responders (orange lines); and responders (blue lines). Panels B and C, are graphical representation showing representative growth curves of mice with bilaterally inoculated AB1 (B) and Renca (C) tumours, treated with ICB therapy (anti-CTLA4/anti-PD-L1); (n=10) pooled data from 2 independent experiments, colour-coded per mouse. Panel D, is a schematic representation showing the experimental design of according to the present invention as outlined in Examples 1. Panels E and F, are three dimensional graphical representations showing PCA clusters of responsive (RS) and non-responsive (NR) AB1 tumours (E) and Recta (F) tumours (n=12 per group). Panels G and H, show a graphical representation of unsupervised hierarchical clustering of top differentially expressed genes clearly separating responsive and non-responsive tumours. For AB1 (G), 10307 genes were differentially expressed in responders versus non-responders (top 200 shown), and 127 genes for Renca (H) (all shown, also see FIG. 2). Panel I, Flow cytometric validation of increased PD-L1 expression on the protein level in responders.

FIG. 2 shows graphical representations of Individual Gene Set Enrichment Analysis graphs of the top 8 Hallmark gene sets in AB1 and Renca tumours. Individual random walk graphs from both AB1 (Panel A) and Renca (Panel B) showing a strong association of these hallmark gene signatures in responders. In the upper right corner of each graph: NES=Normalised enrichment score, ES=Enrichment Score, FDR=False discovery rate q-score.

FIG. 3 demonstrates checkpoint blockade responsive tumours display an inflammatory microenvironment, driven by STAT1, whereby inflammatory pathways with STAT1 as a key regulator are enriched in ICB responsive tumours in mouse models and patients. Panel A, is a graphical representation showing GSEA analysis of top hallmark gene sets in responsive versus non-responsive AB1 and Renca tumours. Panel B, is a graphical representation showing GSEA analysis of responsive versus non-responsive tumours from a patient cohort (n=192) (S. Mariathasan et al., (2018) Nature 554, 544-548). Panel C, is a graphical representation showing Ingenuity pathway analysis displaying canonical pathways enriched in responding mice, combined data from AB1 and Renca tumours. Panel D, is a graphical representation showing coexpression modules which were identified using WGCNA and related to treatment response by identifying differentially expressed genes between responsive and non-responsive tumours and plotting the differential t-statistics as box-and-whisker plots on a module-by-module basis. Bars=SD with outliers, and the dashed horizontal line correspond to p=0.05. Panel E, shows prior knowledge-based graphical reconstruction of the wiring diagram of the module 1. Panel F, is a graphical representation showing GSEA analysis of responsive (CR) versus non-responsive (PD) tumours from the patient cohort (n=192) (S. Mariathasan et al., (2018) Nature 554, 544-548), using a STAT1 gene set (M. A. Care et al., (2015) Genome Med 7, 96). Panel G, is a pictorial representation showing representative pSTAT1 immunohistochemistry in non-responsive and responsive AB1 tumours. Panel H, is a graphical representation survival curves of pSTAT1 high and low ICB treated tumours (n=10 responders (RS), 8 non-responders (NR); Logrank test, *p<0.05).

FIG. 4 is a graphical representation of weighted gene correlation network analysis, which identified seven modules of highly differentially co-expressed genes operating within AB1 and Renca tumours between responsive and non-responsive tumours. Network modules overlayed onto p-values from DESeq2 analysis comparing responders to non-responders revealed which modules were associated with response. The results indicate that expression of modules is upregulated in responders in AB1 and Renca separately. AB1 tumours (Panel A) demonstrated an overwhelming large differential expression in responders compared to non-responders, as reflected in 5 out of 7 modules being significantly associated with response. Renca tumours (Panel B) identified module 1 (brown) as the driver of response. (box and whisker plots, whiskers 1.5 IQR) (dashed horizontal line corresponds to p=0.05).

FIG. 5 is a graphical representation showing Reactome and KEGG pathway enrichment of response-associated module (module 1) in overlap AB1/Renca. Pathway analysis reveals immune-associated pathways, including NK cell mediated cytotoxicity, reflected by the genes upregulated in the response associated module (dashed line corresponds to p=0.05).

FIG. 6 is a graphical representation showing a single cell analysis of responsive/non-responsive AB1 tumours. Panel A, shows transcriptomes of responsive and non-responsive AB1 tumours visualized by tSNE (10,743 cells). Cell subsets were annotated using SingleR. Panel B, is a Violin plot of STAT1 expression across cell types in responsive and non-responsive AB1 tumours.

FIG. 7 demonstrates STAT1 expression and correlation with response in human patient cohorts. Panel A, is a graphical representation showing STAT1 expression in responders (CR and PR) and non-responders (SD and PD) in urothelial cancer patients treated with anti-PD-L1 (Mariathasan et al., 2018; n=298 with response data). Panel B, inventors also tested the STAT1 signature in a second cohort, of melanoma patients treated with the anti-PD1 antibody nivolumab (Riaz et al. 2017). The results are GSEA analysis of responsive (CR and PR=10) versus non-responsive (PD=23) tumours from a melanoma patient cohort treated with anti-PD1 (n=33), using a STAT1 gene set as described in Example 2. Panel C, since we identified that phosphorylated STAT1 correlated with response to ICB in mice, and in absence of available prospectively collected tumour tissue from human patients, the inventors sought to identify whether a STAT1 activation signature would be associated with response. All patients (n=348) were divided into two even groups based on their level of STAT1 activation. High STAT1 activation, as defined by a STAT1 gene set using the Classification of Biological Signatures algorithm (see materials and methods in Example 2), correlated with increased overall survival regardless of radiological response. CR, complete response; PR, partial response; SD, stable disease; PD, progressive disease.

FIG. 8 demonstrates STAT1 immunohistochemistry conducted on AB1 and Renca tumours. Panel A, is a pictorial representation showing a representative immunohistochemical staining of total STAT1 in non-responsive (left) and responsive (right) Renca tumours. The total STAT1 staining was more difficult to assess than the pSTAT1 staining, due to STAT1 having less variability in percentage of positive cells between samples. Panel B, outlines the survival curves of STAT1 positive and negative Renca and AB1-bearing mice treated with anti-CTLA4/anti-PD-L1 (n=9 responders, 7 non-responders; Logrank test, *p<0.05).

FIG. 9 demonstrates that NK cells are enriched in ICB sensitive tumours in mouse models and patients and are required for response to ICB therapy. Panel A, is a graphical representation of results obtained from flow cytometry (1200 merged events) of dissociated tumours from responsive (n=6) and non-responsive (n=9) AB1 tumour, or responsive (n=7) and non-responsive (n=11) Renca tumour-bearing mice. Panel B, is CIBERSORT analysis of RNAseq data from responsive and non-responsive AB1 or Renca tumours (n=12/group). Panel C, is a graphical representation showing percentage (%) of NK cells (CD335+) in responsive and non-responsive tumours shown in panel A. Panel D, is a graphical representation showing NK fraction relative to total leukocyte infiltrate by CIBERSORT (Mann-Whitney U test with Benjamini-Hochberg correction for multiple comparisons). Panel E, shows activated NK cell fraction (CIBERSORT) in tumors from the patient cohort. CR, complete response, PR, partial response, SD, stable disease; PD, progressive disease. (n=298, Mann-Whitney U test, bars=standard deviation). Panels F and G, are Survival plots of AB1 (F) or Renca-bearing mice (G) treated with ICB, with or without the NK depleting antibody antiasialo-GM1 (aGM1) 3 days prior to start of ICB. Mice were treated early with ICB (day 5 for AB1, day 7 for Renca), allowing interrogation of response attenuation (n=10 mice/group, pooled data from 2 independent experiments, Logrank test). *p<0.05, **p<0.01, ***p<0.001.

FIG. 10 is a graphical representation showing Individual samples CIBERSORT in responders and non-responders in AB1 and Renca before treatment with anti-CTLA4 antibody/anti-PD-L1 antibody. Stacked graphs of individual samples of whole AB1 (Panel A) and Renca (Panel B) tumours were analyzed by CIBERSORT using RNA sequencing data. 25 cell subsets were discriminated as a relative proportion of the leukocytes within each sample. The two models demonstrated clear differences in cellular composition. Moreover, there was variability between individual mice, both within and between the responsive and non-responsive groups.

FIG. 11 is a CIBERSORT stacked graph of overall cell populations in Atezolizumab treated patient cohort. CIBERSORT cell subsets were condensed into 9 key subsets and plotted as a stacked graph per response type (CR, Complete Response; PR, Partial Response; SD, Stable Disease; PD, Progressive Disease). There were no significant differences between the cell populations between any groups, except for an increase in NK cells (see FIG. 9).

FIG. 12 shows graphical representations of upstream regulator analysis of differentially expressed genes in AB1, Renca tumours and module 1 (combined AB1 and Renca) by p-value and z-score. Both AB1 (Panels A and D) and Renca (Panels B and E) URA resulted in similar target outputs. The module 1 URA (Panels C and F) also resulted in a similar output, reinforcing this module is central to the response. All 3 analyses identified IFNγ and Poly(I:C) as top positive regulators, and II-10 as a top negative regulator. URA of the overlap of DE genes (AB1 and Renca) by p-value shown in FIG. 13A, and by z-score (Panel G) (Positive regulators in red, negative regulators in blue (by z-score); dashed line=p=0.05).

FIG. 13 demonstrates therapeutic modulation of the tumour microenvironment to promote or enhance sensitization of tumours to ICB therapy. Panel A, is a graphical representation of Upstream Regulator Analysis (URA) of combined data from AB1 and Renca tumours, showing top predicted regulators of the response-associated gene signature, ranked by p-value (red is positive correlation, blue is negative correlation). Panel B, is a graphical representation of URA of predicted regulators in responders versus non-responders of the patient cohort (n=192) (S. Mariathasan et al., (2018) Nature 554, 544-548). Panel C, is a schematic representation showing a non-limiting sensitizing treatment schedule employed in the present invention. Panels D-G, are survival curves of BALB/c mice bearing Renca kidney cancer tumours—(D), BALB/c mice bearing AB1 mesothelioma tumours—(E), C57BL/6 mice bearing AE17 mesothelioma tumours—(F) and C57BL/6 mice bearing B16 melanoma tumours (G) treated for three days with a combination of agents (IFNγ, Poly-I:C, anti-IL-10 antibody) predicted by the inventors to sensitise tumour response blockade therapy followed by ICB (followed by treatment (anti-CTLA4/anti-PD-L1) compared to ICB alone (n=10/group of mice; 15 for AE17 tumour bearing mice) or versus vehicle controls (PBS). Panel H, shows survival curves of Renca tumours bearing mice treated with single agent therapy (anti-II10 antibody or Poly(I:C) or IFNγ) versus the triple combination prior to ICB. Panel I, shows survival curves of Renca tumours-bearing mice treated with double agent therapy versus the triple combination prior to ICB (n=10/group, 2 independent experiments of 5 mice/group; Logrank test compared to ICB only group, *p<0.05, **p<0.01, ***p<0.001). Panel J, is a diagram showing treatment schedule to test ICB followed by sensitising agents for example triple combination therapy. Panel K, shows survival curves of Renca-bearing mice treated with ICB followed by triple combination therapy, as shown in J, or with the reverse schedule, as shown in panel C (n=10/group, 2 independent experiments of 5 mice/group) (Logrank test, *p<0.05, **p<0.01). Panel L, shows survival curves of Renca tumour-bearing mice pretreated with a CD40 agonistic antibody, Poly(I:C) and IL-10, followed by anti-CTLA4/anti-PD-L1 as ICB treatment (CPB).

FIG. 14 demonstrates URA analysis of some negative regulators in the database from a cohort of patients with of urothelial cancer treated with Atezolizumab as ICB, identifying several negative upstream regulators (dashed line=p=0.05).

FIG. 15 survival curves of AB1 tumours-bearing mice treated with a triple agent combination (anti-II10 antibody, Poly(I:C) and IFNγ) followed by anti-CTLA4/anti-PD-L1 treatment as ICB versus treatment of Renca tumours-bearing mice first with ICB and then followed by treatment with the same triple combination (i.e., reverse treatment schedule). To determine whether the triple combination was sensitizing the tumour to ICB, rather than enforcing the effector response, a schedule where the triple combination was followed by ICB has been compared to to a schedule where the ICB followed by the triple combination. ICB treatment only, triple combination only and PCS behicle controls were also analysed. In each mice group n=5/group (Logrank test, *p<0.05, **p<0.01).

FIG. 16 is a graphical representation showing tumour growth curve of AE17 mesothelioma tumour-bearing mice that were pretreated with poly(I:C) for 3 days (or PBS as a control), followed by immune checkpoint blockade (ICB) with anti-CTLA4/anti-PD-L1, with or without an antibody that blocks the IFN alpha/beta receptor (IFNAR). Pretreating with poly(I:C) before the ICB gives a clear delay in tumour growth compared to ICB alone, but this is abrogated by blocking the IFNAR, demonstrating that the tumour sensitising effect of poly(I:C) is mediated through the induction of IFN alpha/beta signalling and that the sensitising effect of poly(I:C) on the tumour microenvironment was abolished by blocking (IFNAR). In particular this data demonstrates that induction of interferon alpha/beta signalling also promotes or enhances sensitivity of tumours to ICB. ICB treatment is denoted on the graph as “CPB”.

FIG. 17 is a graphical representation showing flow cytometric analysis of AE17 tumours after pretreatment with IFN alpha, poly(I:C) or PBS control. AE17 mesothelioma tumour-bearing mice were pretreated with poly(I:C) or recombinant IFN alpha intratumourally for 3 days (or PBS control), after which tumours were dissociated and stained for NK marker CD335, pan-leukocyte marker CD45 and pSTAT1. The results show (a) percentage of NK cells of all leukocytes, (b) percentage of pSTAT1⁺ leukocytes and (c) percentage of pSTAT1⁺ NK cells. These results demonstrate that recombinant IFN alpha induces in the cellular microenvironment of the tumour a an ICB response-associated profile similar to poly(I:C) which is characterized at least by increased NK cell numbers (i.e., increased NK cells infiltration) and pSTAT1 activation (i.e., phosphorylation of STAT1).

FIG. 18 shows that therapeutic modulation of the tumour microenvironment sensitizes tumours to ICB. Panels A-D, are graphical representations showing NK cell fraction (B), STAT1 activation (B), and IIFNγ production (C) by CD45⁺ tumour-infiltrating leukocytes, and PD-L1 expression by CD45⁻ non-leukocytes (D) after treatment with IFNγ, Poly(I:C) and anti-IL-10 (Mann-Whitney U test, bars=standard deviation, *p<0.05, **p<0.01, ***p<0.001). Panels E and F, are graphical representations showing, IFNγ expression (E) and STAT1 phosphorylation (F) in tumour-infiltrating lymphocytes (CD45⁺ cells) (Mann-Whitney U test) *p<0.05, **p<0.01. Panel G, shows survival curves of Renca-bearing mice, receiving sensitization followed by ICB, with or without NK-depleting anti-asialo-GM1 antibody (n=10/group; n=5 PBS controls; Logrank test, ***p<0.001). Panel H, is a schematic representation outlining an exemplary two-step approach to treating cancer patients according to invention, in which tumour profiling enables a decision to treat initially with ICB or after treatment with one or more sensitizing agents e.g., such as a triple combination of agents exemplified in the proceeding Figures.

FIG. 19 Treatment with IFNγ+Poly(I:C)+anti-II-10 antibody phenocopies a response-associated tumour microenvironment. To determine if the predicted upstream regulators could induce a responder phenotype, inventors analysed treated tumours by flow cytometry. Treating large established tumours with IFNγ, Poly(I:C) and anti-II-10 resulted in a phenotype similar to pre-treatment responsive tumours. (Panel A) Treatment increased the proportion of NK cells infiltrating the tumour, characteristic of a responsive tumour (see FIG. 9). Inventors also observed increased monocytes, and decreased CD4⁺ T cells and dendritic cells (n=9 per group, 2 individual experiments, Renca). (Panel B) In addition to increased pSTAT1 on CD45⁺ leukocytes (FIG. 18B), inventors also observed increased pSTAT1 expression in non-leukocytes (CD45⁻ i.e. tumour cells and stroma) after treatment (Mann-Whitney U test) *p<0.05, **p<0.01.

FIG. 20 demonstrates that CD335⁺ cells in tumours are conventional NK cells. To determine the phenotype of CD335⁺ cells in tumours inventors performed flow cytometric analysis of live CD45⁺ CD3⁻ TCRb− CD19− CD335⁺ cells (Panel A). The CD335⁺ population primarily expressed the NK cell-specific markers Eomes and CD49b. Cells also expressed T-bet, while lacking the ILC3 marker Rorγt and the ILC1 markers CD127 and CD200r (Panel B). Plots in (A) and (B) are representative of both untreated (n=4) and treated (n=5) tumours. Representative samples (black open histogram) are overlayed with staining controls (grey tinted histogram) for each marker.

FIG. 21 provides graphical representations of tumour growth curves of AB1-HA tumour bearing mice were treated with checkpoint blockade to (ICB) antibodies using anti-CTLA4 antibody and anti-PD-L1 antibody (anti-CTLA4/anti-PD-L1) on day 7, 9, 11 in combination with tretinoin given for 9 days starting three days earlier on day 4 (panels C, D, E) or concomitantly on day 7 (panel F), in increasing dosages (panels C, D, E). Efficacy was compared with ICB alone (panel B) and to PBS control (panel A). CPB=ICB treatment. The results demonstrates that response to ICB was improved by administration of a retinoid such as tretinoin.

FIG. 22 demonstrates that pretreatment with a retinoid, for example, tretinoin, induces increased STAT1 activation in tumours. Panels A and B show immunohistochemistry staining for phospho-STAT1 on tumours from CT26 colorectal cancer-bearing mice that were treated with tretinoin via oral gavage for 5 days. Panel C is a graphical representation the number of pSTAT1 positive MHCII⁺ cells as percentage of all tumour-infiltrating leukocytes, measured by flow cytometry in Renca tumours after 3 days of i.p. treatment with tretinoin. The graph plots show the number of pSTAT1 positive MHCII⁺ cells as percentage of all tumour-infiltrating leukocytes, measured by flow cytometry, in AB1 tumours after treatment of mice with tretinoin.

FIG. 23 is a tumour growth curve of AB1-HA tumours in mice after treatment of mice with anti-PD-L1 antibody (ICB therapy) with or without tretinoin or vehicle control (PBS). Tretinoin was given daily for 9 days at 10 mg/kg i.p., starting on day 6 after tumour inoculation (grey shaded area), the anti-PD-L1 antibody was given on days 8, 10 and 12 at 200 μg/mouse. The results demonstrate that combination treatment with tretinoin and an antibody blocking the PD-1/PD-L1 axis is more efficacious than use of the antibody alone.

FIG. 24. Shows tumour growth curves of Renca kidney cancer tumours in mice that were treated with ICB (anti-CTLA4 antibody and anti-PD-L1 antibody=demoted as CBP on the graph) alone (panel A), or in combination with bexarotene (panel B), or in combination with tretinoin (panel C), or in combination with isotretinoin (panel D). Panel E shows survival curves of all treatment groups. ICB (anti-CTLA4 antibody and anti-PD-L antibody) was given on day 12,14 and 16, and the retinoid was given orally for 6 days (grey shaded area), starting on day 9. The results obtained demonstrate that different retinoid compounds/drugs are able to sensitise tumours to ICB therapy.

FIG. 25 Shows a schematic graphical representation of the flow cytometry gating strategy employed in the working examples (see e.g., Example 2, part 8).

FIG. 26 is a tabular representation of the antibodies used in the flow cytometry method employed in the working examples (see e.g., Example 2, part 8).

FIG. 27 provides a list of the 118 genes found to be differentially expressed in both Renca tumours and AB1 tumours a referred to in Example 4.

### DETAILED DESCRIPTION OF THE PREFERRED EMBODIMENTS

According to the invention the inventors have revealed that one or more immune checkpoint sensitising agents and preferably a combination thereof attract effector immune cells and in particular IFNγ producing NK cells into a tumour environment inducing tumour cell sensitisation enhancing the efficacy of immune checkpoint blockade agents on a malignant condition. That is, the cellular constituents of a tumour can be targeted by immune checkpoint sensitising agents causing immune effector cells to be stimulated within a tumour microenvironment. When this is achieved a tumour becomes sensitized to immune checkpoint blockade agents.

For convenience, Section 1, below, outline the meanings of various terms used herein. Section 2, which follows, presents a general description of the invention as it realates to methods of use, use of medicaments and methods of manufacturing medicaments are discussed. This section of the description is supported by specific examples demonstrating the properties of various embodiments of the invention and how they can be employed. Each example, embodiment and aspect described herein is to be applied mutatis mutandis to each and every other example, embodiment and aspect unless specifically stated otherwise.

1. Definitions

The meaning of certain terms and phrases used in the specification, examples, and appended claims, are provided below. If there is an apparent discrepancy between the usage of a term in the art and its definition provided herein, the definition provided within the specification shall prevail.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however be understood to be common general knowledge.

Manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

Other than in the operating examples, or where otherwise indicated, all numbers expressing quantities of ingredients or reaction conditions used herein should be understood as modified in all instances by the term “about.” The term “about” when used in connection with percentages can mean ±1%.

The invention described herein may include one or more range of values (e.g. size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this specification, the use of the singular includes the plural unless specifically stated otherwise. In this application, the use of “or” means “and/or” unless stated otherwise. Furthermore, the use of the term “including”, as well as other forms, such as “includes” and “included”, is not limiting.

Also, terms such as “element” or “component” encompass both elements and components comprising one unit and elements and components that comprise more than one subunit unless specifically stated otherwise.

Throughout this specification, the phrase “immune checkpoint blockade agent(s)” includes without limitation, an agent that targets the inhibitory T cell molecule CTLA-4 and/or targets the Programmed Death receptor (PD-1) and/or PD-Ligand (PD-L) pathway and/or glucocorticoid-induced tumour necrosis factor receptor (GITR) and/or LAG3 and/or OX40 and/or 41BB and/or TIM3. An example of an agent that targets the inhibitory T cell molecule CTLA-4 is a CTLA-4 antagonist such as ipilimumab or tremelimumab. An example of an agent that targets PD-1 is a PD-1 antagonist such as nivolumab, AMP-224, pidilizumab, spartalizumab, cemiplimab, camrelizumab or pembrolizumab. An example of an agent that targets PD-L1 is a PD-L1 antagonist such as Atezolizumab, Avelumab or Durvalumab. Examples of agents that target GITR are antagonists such as TRX518 or MK4166. Examples of agents that target LAG3 are BMS-986016, BI 754111, LAG-525 or REGN-3767. An example of an agent that targets OX40 is BMS 986178, MED16469, GSK3174998, PF-04518600. Examples of agents that target TIM3 are LY3321367, MBG453 or TSR-022. An example of an agent that targets 41BB is PF-05082566.

Throughout this specification, the phrase “immune checkpoint sensitising agent(s)” includes, without limitation agents selected from the group comprising: a CD40 agonist, an anti-IL10 antibody, an inducer of interferon alpha/beta signalling, an interferon gamma or a functional variant thereof and/or a retinoid. For example, the immune checkpoint sensitising agent(s) include without limitation an agonistic CD40 antibody, anti-IL10, Poly(I:C), a retinoid (such as all-trans retinoic acid) and/or Interferon gamma. Preferably the phrase means a combination of immune checkpoint sensitising agents, said combination being at least a plurality of the identified sensitising agents selected from the group comprising: an agonistic CD40 antibody, anti-IL10, Poly(I:C), a retinoid (such as all-trans retinoic acid) and/or Interferon gamma. Ideally, the combination will be a combination of at least three of the following agents anti-IL10, Poly(I:C) and interferon gamma or anti-CD40, anti-IL10, or a retinoid such as all-trans retinoic acid and interferon gamma.

For It will be understood that the term “CD40 agonist” encompasses without limitation agonistic CD40 antibodies or a CD40 ligands. Examples of agonistic CD40 antibodies suitable for use in the present invention include but are not limited to dacetuzumab (also known as SGN-40, e.g., by Seattle Genetics, Inc.) CP-870,893 (e.g., Pfizer), ChiLob7/4 (e.g., by University of Southampton), ABBV-927 (e.g., by Abbvie), APX005M (e.g., by Apexigen, Inc.). Exemplary CD40 ligand suitable for use in the present invention includes but not limited to a recombinant human CD40 ligand such as rhuCD40L (e.g., by Immunex Corp).

As used herein, the terms “anti-IL10 antibody” will be understood to include any antibody that targets the IL-10 receptor and which antagonises and/or abolishes activity or IL-10 receptor e.g., in a neoplastic cell and/or a neoplastic tumour. Exemplary anti-IL10 antibodies suitable for use in the present invention include but are not limited to MK-1966 (e.g., by Merck) and/or BT-063 (e.g., by Biotest).

As used herein, “inducer of interferon alpha/beta signalling” will be understood to include any compound, drug, or composition which is capable of inducing and/or enhancing IFN alpha/beta signalling such as by induction and/or activation of the IFN alpha/beta receptor (IFNAR) in a cell such as a neoplastic cell and/or a tumour cell. Suitable inducers of interferon alpha/beta signalling include but are not limited to toll-like receptor 3 (TLR3) ligands. For example, suitable TLR3 ligands suitable for use in the present invention include poly(I:C) (i.e., olyinosinic:polycytidylic acid); poly(A:U) (i.e., polyadenylic-polyuridylic acid), polyl:polyC12U (i.e., rintatolimod); poly ICLC (i.e., 4-aminobutylcarbamic acid; [5-(4-amino-2-oxopyrimidin-1-yl)-3,4-dihydroxyoxolan-2-yl]methyl dihydrogen phosphate; [3,4-dihydroxy-5-(6-oxo-3H-purin-9-yl)oxolan-2-yl]methyl dihydrogen phosphate; 2-hydroxyacetic acid (a synthetic complex of carboxymethylcellulose, polyinosinic-polycytidylic acid, and poly-L-lysine double-stranded RNA); and/or the synthetic dsRNA conjugated to phosphorothioate oligodeoxynucleotide known as “sODN-dsRNA” (M. Matsumoto et al., (2015) Nat Commun, 6 p. 6280).

As used herein the term “an interferon gamma or functional variant thereof” includes any IFNγ cytokine such as a human IFNγ or any functional variant or fragment thereof such as a recombinant human IFNγ cytokine capable of activating STAT1 e.g., in a neoplastic cell such as a tumour cell. Suitable human IFNγ variant includes but are not limited to a recombinant human IFNγ 1b.

Retinoids which are suitable for use in the present invention include but are not limited to tretinoin (also known as all-trans retinoic acid or retinoic acid) retinol, retinal, isotretinoin (13-cis-retinoic acid), alitretinoin (9-cis-retinoic acid), etretinate, acitretin, adapalene, bexarotene, tazarotene. Preferably, the retinoids include tretinoin and/or bexarotene and/or isotretinoin. For example, the retinoids includes any two or more of tretinoin and/or bexarotene and/or isotretinoin. Preferably, at least tretinoin is employed in the present invention.

As used herein, the term “neoplastic cell” or “neoplastic cell population” will be understood to refer to cells or cell populations demonstrating abnormal and/or excessive and/or or uncontrolled growth. For example a neoplastic cell or cell population will be uncoordinated with that of the normal body surrounding tissue, and may persist growing abnormally and/or excessively even when the original trigger which induced abnormal and/or excessive growth behaviour is removed from said cell or cell population. It will be understood that the abnormal and/or excessive growth of the neoplastic cell or cell population may result in formation of cells mass such as a tumour. It will also be understood that the term neoplastic cell or neoplastic cell population encompass cells or cell populations which are malignant or benign. In one preferred example the neoplastic cell or neoplastic cell population is malignant such as a cancerous tumour. In one particularly preferred example, the neoplastic cell or cell population according to any aspect, embodiment, form or example described herein throughout is capable of activating or phosphorylating STA1 protein and/or is capable of producing IFNγ cytokine.

In one example, the neoplastic cell or cell population according to any aspect, embodiment, form or example of the invention as described herein throughout is malignant or benign.

In one example, when neoplastic cell or cell population is malignant it may comprise one or more cancer tumour cells selected from Acute Lymphoblastic Leukemia (ALL), Acute Myeloid Leukemia (AML), Adrenocortical Carcinoma, Anal Cancer, Astrocytomas, Atypical Teratoid/Rhabdoid tumour, Basal Cell Carcinoma, Bile Duct Cancer, Bladder Cancer, Bone Cancer (includes Ewing Sarcoma and Osteosarcoma and Malignant Fibrous Histiocytoma), Brain tumours, Breast Cancer, Bronchial tumours, Carcinoid tumour, Carcinoma of Unknown Primary, Cervical Cancer, Chronic Lymphocytic Leukemia (CLL), Chronic Myelogenous Leukemia (CML), Chronic Myeloproliferative Neoplasms, Colorectal Cancer, Cutaneous T-Cell Lymphoma, Ductal Carcinoma In Situ (DCIS), Embryonal tumours, Endometrial Cancer, Esophageal Cancer, Esthesioneuroblastoma, Extracranial Germ Cell tumour, Extragonadal Germ Cell tumour, Eye Cancer, Retinoblastoma, Fallopian Tube Cancer, Gallbladder Cancer, Gastric (Stomach) Cancer, Gastrointestinal Carcinoid tumour, Gastrointestinal Stromal tumours (GIST), Childhood Central Nervous System Germ Cell tumours, Childhood Extracranial Germ Cell tumours, Extragonadal Germ Cell tumours, Ovarian Germ Cell tumours, Testicular Cancer, Gestational Trophoblastic Disease, Hairy Cell Leukemia, Head and Neck Cancer, Heart tumours, Hepatocellular (Liver) Cancer, Histiocytosis, Hodgkin Lymphoma, Hypopharyngeal Cancer, Intraocular Melanoma, Islet Cell tumours, Pancreatic Neuroendocrine tumours, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone ,Melanoma, Merkel Cell Carcinoma, Mesothelioma, Malignant, Metastatic Cancer, Metastatic Squamous Neck Cancer with Occult Primary, Midline Tract Carcinoma With NUT Gene Changes, Mouth Cancer, Multiple Endocrine Neoplasia Syndromes , Multiple Myeloma or Plasma Cell Neoplasms, Mycosis Fungoides, Myelodysplastic Syndromes, Myelodysplastic or Myeloproliferative Neoplasms, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine tumours, Papillomatosis, Paraganglioma, Paranasal Sinus and Nasal Cavity Cancer, Parathyroid Cancer tumours, Penile Cancer tumours, Pharyngeal Cancer tumours, Pheochromocytoma, Pituitary tumour, Pleuropulmonary tumours, Primary Central Nervous System (CNS) Lymphoma tumours, Primary Peritoneal Cancer tumours, Prostate Cancer tumours, Rectal Cancer tumours, Renal Cell (Kidney) Cancer tumours, Retinoblastoma tumours, Rhabdomyosarcoma tumours, Salivary Gland Cancer tumours, Sarcoma tumours, Vascular tumours , Ewing Sarcoma tumours, Kaposi Sarcoma tumours, Soft Tissue Sarcoma tumours, Uterine Sarcoma tumours, Sezary Syndrome tumours, Skin Cancer tumours, Small Cell Lung Cancer tumours, Small Intestine Cancer tumours, Squamous Cell Carcinoma of the Skin tumours, Squamous Neck Cancer with Occult Primary, Metastatic tumours, Stomach (Gastric) Cancer tumours, T-Cell Lymphoma, Cutaneous, Testicular Cancer tumours, Nasopharyngeal Cancer tumours, Oropharyngeal Cancer tumours, Hypopharyngeal Cancer tumours, Thymoma and Thymic Carcinoma tumours, Thyroid Cancer tumours, Transitional Cell Cancer of the Renal Pelvis and Ureter tumours, Ureter and Renal Pelvis tumours, Transitional Cell Cancer, Urethral Cancer tumours, Uterine Cancer tumours, Endometrial Uterine Sarcoma tumours, Vaginal Cancer tumours, Vulvar Cancer tumours, Wilms tumour and Childhood Kidney tumours, and/or mesothelioma tumours.

As used herein, the term “neoplastic tumour” or “tumour” will be understood to refer to abnormal mass of cells or tissue showing uncoordinated abnormal and/or excessive growth relative to that of the normal body surrounding tissue(s) and includes neoplastic cell and other cells including but not limited to stromal and immune cells such lymphocytes (e.g., NJK cells) T cells and dendritic cells. A neoplastic tumour or tumour according to any aspect, embodiment, form or example of the invention described herein throughout preferably include neoplastic cells capable of activating or phosphorylating STA1 protein and/or is capable of producing IFNγ cytokine. It will also be understood that a “neoplastic tumour” or “tumour” as used herein may be benign or malignant. Preferably the tumour is a malignant tumour.

In one example the tumour may be a malignant tumour selected from: Acute Lymphoblastic Leukemia (ALL) tumour, Acute Myeloid Leukemia (AML) tumour, Adrenocortical Carcinoma, Anal Cancer, Astrocytomas, Atypical Teratoid/Rhabdoid tumour, Basal Cell Carcinoma, Bile Duct Cancer, Bladder Cancer, Bone Cancer (includes Ewing Sarcoma and Osteosarcoma and Malignant Fibrous Histiocytoma), Brain tumours, Breast Cancer, Bronchial tumours, Carcinoid tumour, Carcinoma of Unknown Primary, Cervical Cancer, Chronic Lymphocytic Leukemia (CLL), Chronic Myelogenous Leukemia (CML), Chronic Myeloproliferative Neoplasms, Colorectal Cancer, Cutaneous T-Cell Lymphoma, Ductal Carcinoma In Situ (DCIS), Embryonal tumours, Endometrial Cancer, Esophageal Cancer, Esthesioneuroblastoma, Extracranial Germ Cell tumour, Extragonadal Germ Cell tumour, Eye Cancer, Retinoblastoma, Fallopian Tube Cancer, Gallbladder Cancer, Gastric (Stomach) Cancer, Gastrointestinal Carcinoid tumour, Gastrointestinal Stromal tumours (GIST), Childhood Central Nervous System Germ Cell tumours, Childhood Extracranial Germ Cell tumours, Extragonadal Germ Cell tumours, Ovarian Germ Cell tumours, Testicular Cancer, Gestational Trophoblastic Disease, Hairy Cell Leukemia, Head and Neck Cancer, Heart tumours, Hepatocellular (Liver) Cancer, Histiocytosis, Hodgkin Lymphoma, Hypopharyngeal Cancer, Intraocular Melanoma, Islet Cell tumours, Pancreatic Neuroendocrine tumours, Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone, Melanoma, Merkel Cell Carcinoma, Mesothelioma, Malignant, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma or Plasma Cell Neoplasms, Mycosis Fungoides, Myelodysplastic Syndromes, Myelodysplastic or Myeloproliferative Neoplasms, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine tumours, Papillomatosis, Paraganglioma, Paranasal Sinus and Nasal Cavity Cancer, Parathyroid Cancer tumours, Penile Cancer tumours, Pharyngeal Cancer tumours, Pheochromocytoma, Pituitary tumour, Pleuropulmonary tumours, Primary Central Nervous System (CNS) Lymphoma tumours, Primary Peritoneal Cancer tumours, Prostate Cancer tumours, Rectal Cancer tumours, Renal Cell (Kidney) Cancer tumours, Retinoblastoma tumours, Rhabdomyosarcoma tumours, Salivary Gland Cancer tumours, Sarcoma tumours, Vascular tumours, Ewing Sarcoma tumours, Kaposi Sarcoma tumours, Soft Tissue Sarcoma tumours, Uterine Sarcoma tumours, Sezary Syndrome tumours, Skin Cancer tumours, Small Cell Lung Cancer tumours, Small Intestine Cancer tumours, Squamous Cell Carcinoma of the Skin tumours, Squamous Neck Cancer with Occult Primary, Metastatic tumours, Stomach (Gastric) Cancer tumours, T-Cell Lymphoma, Cutaneous, Testicular Cancer tumours, Nasopharyngeal Cancer tumours, Oropharyngeal Cancer tumours, Hypopharyngeal Cancer tumours, Thymoma and Thymic Carcinoma tumours, Thyroid Cancer tumours, Transitional Cell Cancer of the Renal Pelvis and Ureter tumours, Ureter and Renal Pelvis tumours, Transitional Cell Cancer, Urethral Cancer tumours, Uterine Cancer tumours, Endometrial Uterine Sarcoma tumours, Vaginal Cancer tumours, Vulvar Cancer tumours, Wilms tumour and Childhood Kidney tumours, and/or mesothelioma tumours.

For example, the tumour is selected from melanoma tumours, non-small cell lung cancer tumours, Merkel-cell carcinoma tumours, microsatellite instable colorectal cancer tumours, renal cancer tumours, mesothelioma cancer tumours.

The term “microenvironment” as used herein with reference to the microenvironment of a neoplastic cell, cell population and/or neoplastic tumour, will be understood to refer to the local milieu in a mass of the neoplastic cells or cell population or the tumour which includes the neoplastic cell or cell population. It will also be understood that the microenvironment of the neoplastic cell or cell population includes the neoplastic cells in the cell population or tumour and may also include any stromal cells and any immune cells such as lymphocytes e.g., NK cells, T cells and dendritic cells, as well as chemical or immune effectors including but not limited to cytokines e.g., interferons (such as IFNγ, IFNα) and growth factors. It will also be understood that the microenvironment of the neoplastic cell or cell population may also include supportive stromal and vasculature. Ideally, the microenvironment of the neoplastic cell or cell population is permeable to infiltration of NK cells such as STAT1 and IFNγ NK producing NK cells.

Throughout this specification, unless the context requires otherwise, the word “comprise” or variations such as “comprises” or “comprising”, will be understood to imply the inclusion of a stated integer or group of integers but not the exclusion of any other integer or group of integers.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, e.g. in the absence of an agent, for example a decrease by at least about 20%, or at least about 30%, or at least about 40%, or at least about 50%), or at least about 60%>, or at least about 70%, or at least about 80%.

The terms “increased”, ‘increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, e.g. in in the absence of an agent, for example an increase of at least about 20%, or at least about 30%, or at least about 40%, or at least about 50%), or at least about 60%, or at least about 70%, or at least about 80%, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. In certain embodiments, the compound is administered by parenterally administration, or other method allowing delivery to a target site.

In the context of this specification the phrase “effective amount” “therapeutically effective amount” or “effective dose” (used interchangeably herein) includes within its meaning a sufficient but non-toxic amount of a compound or composition of the invention to provide the desired effect. The exact amount of a compound or composition required will vary from subject to subject depending on factors such as the desired effect, the species being treated, the age and general condition of the subject, the severity of the condition being treated, the agent or combination of agents being administered, the mode of administration, and so forth. Thus, it is not possible to specify an exact “effective amount”. However, for any given case, an appropriate effective amount (dose) may be determined by one of ordinary skill in the art using only routine experimentation. Generally, a therapeutically effective amount can vary with the subject's history, age, condition, sex, as well as the severity and type of the medical condition in the subject, and administration of other pharmaceutically active agents.

It is to be noted that reference herein to use in therapeutic applications will be understood to be equally applicable to human and non-human, such as veterinary, applications. Hence it will be understood that, except where otherwise indicated, reference to a “patient”, “subject” or “individual” (used interchangeably herein) means a human or non-human, such as an individual of any species of social, economic or research importance including but not limited to, mammalian, avian, lagomorph, ovine, bovine, equine, porcine, feline, canine, primate and rodent species. More preferably the animal is a mammalian species. The mammalian species is desirably a human or non-human primate or a companion animal such as a domesticated dog, cat, horse, monkey, mouse, rat, rabbit, sheep, goat, cow or pig.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Specific Preferred Embodiments

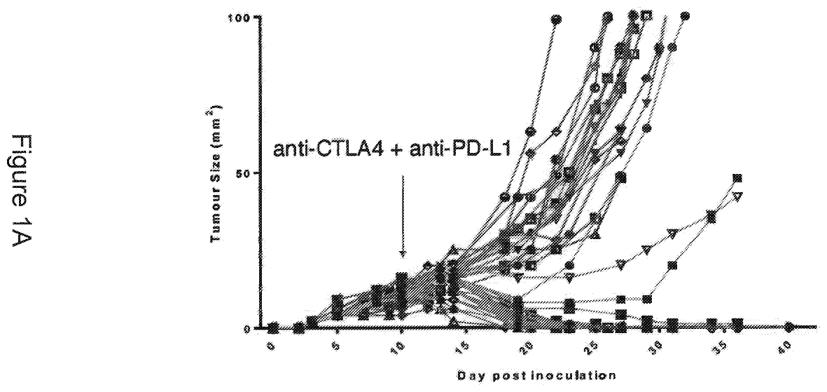

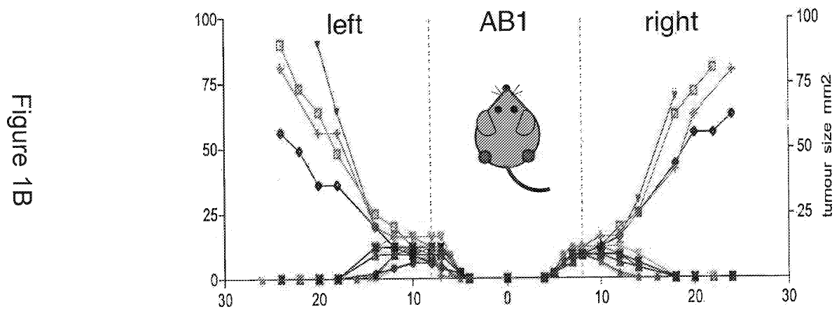

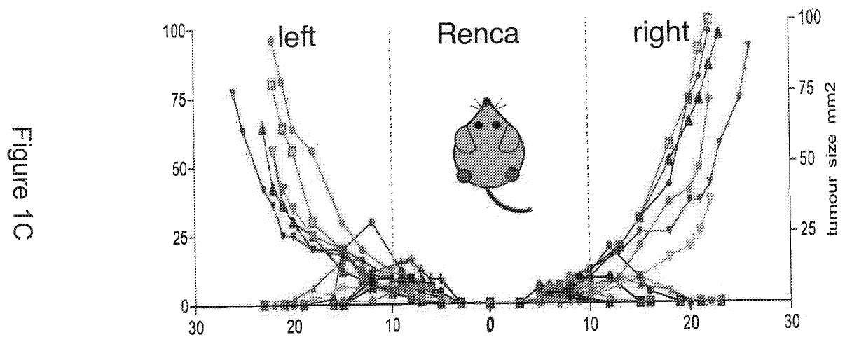

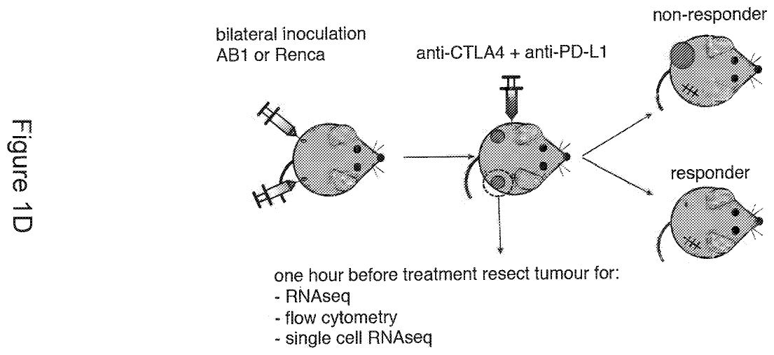

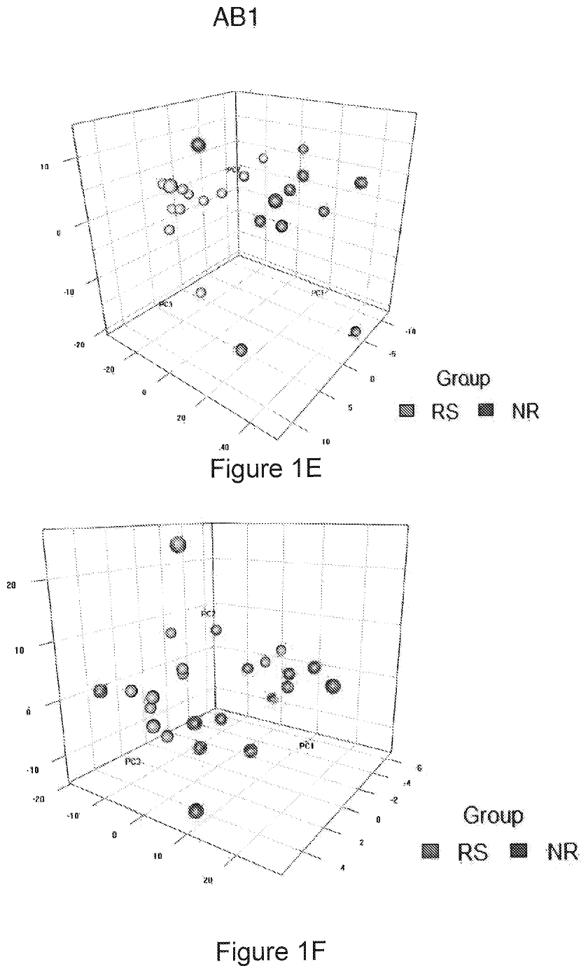

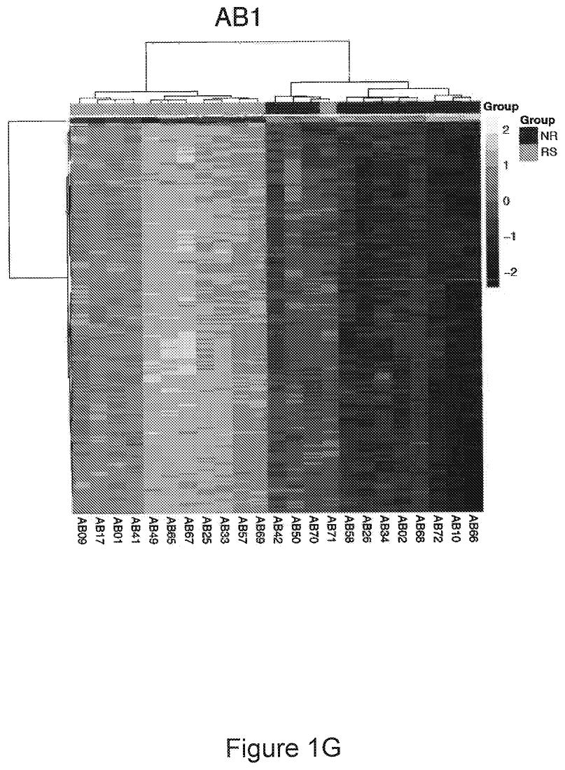

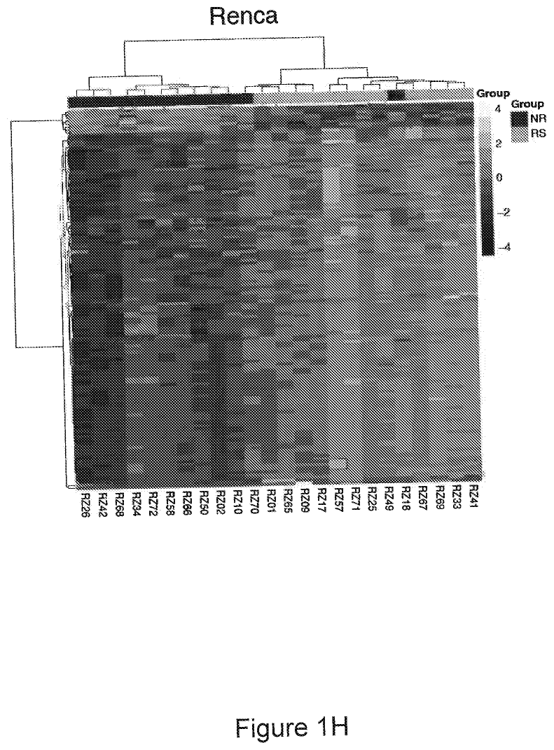

As shown in the working examples that follow, in the work leading to the present invention, the inventors set out to identify the pre-treatment tumour microenvironment associated with sensitivity to immunotherapy with immune checkpoint blockade (ICB) agents. To this effect, the inventors made use of the fact that even in the highly homogeneous setting of inbred mouse strains bearing tumours derived from monoclonal cancer cell lines, there remains a dichotomy in responsiveness to treatment with ICB. Even inbred mouse strains bearing transplantable tumours display a dichotomous outcome after immunotherapy (see for example FIG. 1A). Such dichotomy as observed in the art was surprising since the genomes of these mice were nominally identical and the tumours were derived from a clonal cell line, excluding the possibility that a difference in tumour rejection antigen expression caused these disparate responses. Mice were age- and gender-matched, kept under controlled conditions, and receive identical treatment. Yet, they respond very differently.

Without being bound by any theory or mode of action, the inventors reasoned that differences in outcomes between animals may possibly be related to differences in T cell repertoire, which is not completely encoded in the germline, or to stochastic immunological events. However, regardless of the cause of this dichotomy observed, the inventors reasoned that such dichotomy in response to ICB in inbred mouse strains bearing transplantable tumours derived from a clonal cell line, would allow to assess potentially small differences in microenvironmental regulation of therapeutic responses in a controlled background.

Against this highly uniform background of the dichotomy as observed in the art, the inventors sought to identify a signature in the microenvironment of tumours which exists before the application of immunotherapy treatment with ICB agents and that would correlate with response to the ICB agents. The inventors also sought to use that information for the purpose of promoting or enhancing sensitivity of one or more neoplastic cells and/or tumours to treatment with ICB agents, for example to render non-responders into responders.

To achieve this, and as demonstrated in the working examples that follow, using for example flow cytometry and bulk and single cell RNAseq data from tumours derived from two different mouse cancer models the inventors compared the cellular composition and gene expression profiles of responsive and non-responsive tumours from mice before ICB, and mapped the key cellular and molecular networks associated with response. The results obtained by the inventors corroborated/validated earlier findings obtained using cancer patient cohorts treated with immune checkpoint blockade antibodies targeting the PD-1/PD-L1 pathway (N. Riaz et al., (2017) Cell 171, 934-949 e915; and S. Mariathasan et al., (2018) Nature 554, 544-548).

As shown in the working examples that follow, the inventors prioritised upstream regulators for therapeutic targeting with drugs, recombinant proteins or antibodies, and found surprisingly that indeed it is possible to drive the tumour microenvironment from a non-responsive state to immunotherapy with immune checkpoint blockade agents into a responsive state.

In particular, as shown herein, it was surprisingly found that responsive tumours were characterized inter alia by an inflammatory gene expression signature consistent with upregulation of the signal transducer and activator of transcription 1 protein (STAT1) and the Toll-like receptor 3 (TLR3) signalling, and down-regulation of interleukin 10 (IL-10) signalling. In addition, it was found that responsive tumours had more infiltrating natural killer (NK) cells. Pre-treatment of different mouse strains with large established tumours, using one or more of the STAT1-activating cytokine IFNγ, the TLR3 ligand poly(I:C) and/or an anti-IL-10 antibody sensitized tumors to ICB by attracting IFNγ-producing NK cells into the tumor, resulting in increased cure rates.

The skilled artisan would appreciate that the results presented herein identify a pre-treatment cellular and molecular tumour microenvironment that can predict response to ICB, which can be therapeutically attained. The data presented herein anticipates a biomarker-driven approach to patient management to establish whether or not a patient would benefit from treatment with sensitizing therapeutics before ICB treatment.

As demonstrated in the working examples that follow, the inventors set out a study design to evaluate the neoplastic cellular microenvironment before treatment with immune check point blockade agent(s) associated with an effective outcome by comparing gene expression and flow cytometry data from ICB responsive and non-responsive tumours within the same mouse cancer model. The inventors also sought to demonstrate a method for therapeutically promoting or enhancing the tumour microenvironment towards a responsive phenotype i.e., a phenotype which is susceptible to treatment with ICB agents, and thus increase the response to ICB (see working example 1).

The results shown in example 3 that follows demonstrate that it is possible to differentiate microenvironments of neoplastic cell populations and tumours that were going to be respond to immunotherapy with ICB agents from non-responders even before they were treated with the immunotherapy. Equally, the results also demonstrate that it is possible to differentiate those subjects predicted to be responders to immunotherapy with ICB agents from non-responders. It is also possible to predict whether or not a neoplastic cell population or tumour or a subject having the neoplastic cell population or tumour is going to response to immunotherapy with ICB agents even before treatment with the immunotherapy.

As demonstrated in the work that follows, STAT1 activation is a driver of the ICB response-associated tumour microenvironment and can serve as a potential biomarker to identify neoplastic cell population and/or tumours and/or patients having such neoplastic cell populations and/or tumours more likely to respond to ICB immunotherapy (see e.g., Example 4). As also demonstrated herein, rendering tumours responsive to ICB therapy with one or more sensitising agents can be achieved notwithstanding differences in the cellular tumour microenvironments between different animal models and between different cancer tumours (see e.g., Example 5).

As also shown e.g., in Example 5, infiltration of the tumour microenvironment with NK cells (such as activated NK cells producing IFN gamma and/or STAT1 may contribute to promoting or enhancing sensitivity of neoplastic cells or tumours to ICB therapy.

Using multiple and different mouse tumour models such as models for mesothelioma, kidney cancer and melanoma (which are pathologically entirely distinct cancers) it was also possible to demonstrate application of clinically available therapeutics which result in marked sensitization of neoplastic cell populations and/or tumours to ICB agents which commences prior to the ICB therapy and could be continued during ICB immunotherapy. For example, as shown in the working examples, it was demonstrated that pre-treated with the one or more sensitising agents such as those selected from a CD40 agonist, an anti-IL10 antibody, an inducer of interferon alpha/beta signalling, an interferon gamma or a functional variant thereof and/or a retinoid were able to become sensitised to checkpoint blockade therapy (see e.g., working examples 6,7 and 9). The results show that promoting or enhancing sensitivity of one or more neoplastic cell populations such tumours to ICB is characterised by increased infiltration of activated NK cells (such as those which secrete IFN gamma cytokine) and STAT1 phosphorylation (STAT1 activation) in the tumour cellular microenvironment environment (see e.g., examples 7 and 8). The data obtained outlines a rational for use of one or more sensitising agents, for example two or more of such agents as therapeutics which promote or enhance sensitization of neoplastic cells and/or tumours to ICB. This clears the way to a two-step approach to treating cancer patients where, based on tumour profiling, a decision to treat with ICB can be made initially or after pre-treatment with sensitizing therapeutics (see e,g., examples 6-9).

Accordingly, in one broad aspect this invention relates to methods for sensitizing a cellular population such as neoplastic cell population and/or tumours to the effects of certain immunotherapeutic agents. In particular embodiments, the methods more specifically relate to sensitizing neoplastic cells to the effects of immune checkpoint blockade agents.

Specifically, the invention relies on the identification of sensitising compounds, which when administered in advance of immune checkpoint blockade agents, are able to alter a cellular microenvironment e.g., of tumours causing the cells in that environment to change from being resistant to immune checkpoint blockade agents to being sensitive to those agents.

In one aspect, the invention resides in a method for promoting or enhancing the sensitivity of one or more neoplastic cells and/or neoplastic tumours to immune checkpoint blockade agents, said method comprising the step of: administering to a neoplastic cell and/or a neoplastic tumour, prior to treatment of immune checkpoint blockade agents, one or more immune checkpoint sensitising agents or exposing the cell and/or tumour to the one or more sensitising agents to thereby cause the cells and/or tumour to become sensitized to an immune checkpoint blockade agent.

In a related embodiment of this aspect, there is provided a method for promoting or enhancing the sensitivity of one or more neoplastic cells and/or neoplastic tumours to immune checkpoint blockade agents, said method comprising the step of:
a. administering to a neoplastic cell and/or a neoplastic tumour, prior to treatment of immune checkpoint blockade agents, one or more immune checkpoint sensitising agents, or exposing the cell and/or tumour to the one or more sensitising agents, for a period of time and/or at a therapeutic amount that causes a tumour to become sensitized to an immune checkpoint blockade agent.

In one example, the immune checkpoint sensitising agents are selected from: a CD40 agonist, an anti-IL10 antibody, an inducer of interferon alpha/beta signalling, an interferon gamma or a functional variant thereof and/or a retinoid.

In one such example, the CD40 agonist may be an agonistic CD40 antibody or a CD40 ligand. In one preferred example, the CD40 agonist is an agonistic CD40 antibody.

In another example, the inducer of interferon alpha/beta signalling is a toll-like receptor 3 (TLR3) ligand. For example, the inducer of interferon alpha/beta signalling may be a TLR3 ligand selected from the group consisting of: poly(I:C), poly(A:U), poly ICLC, polyl:polyC12U, and sODN-dsRNA. Preferably, the inducer of interferon alpha/beta signalling is or comprises poly(I:C).

In another example, the retinoid is selected from tretinoin, retinol, retinal, isotretinoin, alitretinoin, etretinate, acitretin, adapalene, bexarotene, and/or tazarotene. Preferably, the retinoid is tretinoin and/or bexarotene and/or isotretinoin. More preferably, the retinoid is tretinoin.

Preferably, the immune checkpoint sensitising agents are selected from the group comprising: an agonistic CD40 antibody, anti-IL10, Poly(I:C), a retinoid and/or Interferon gamma.

In a preferred form of the method, a combination of immune checkpoint sensitising agents are used in the method, said combination being at least a plurality of the identified sensitising agents selected from the group comprising: an agonistic CD40 antibody, anti-IL10, Poly(I:C) , a retinoid (such as all-trans retinoic acid) and/or Interferon gamma. Ideally, the combination will be a combination of at least three of anti-IL10, poly(I:C) and interferon gamma or anti-CD40, anti-IL10, a retinoid such as all-trans retinoic acid combination of a STAT1-activating cytokine IFNγ. For example, the combination can be a STAT1-activating cytokine IFNγ, the TLR3 ligand poly(I:C) and an anti-IL-10 antibody.

Preferably, the sensitising agents are administered to the neoplastic cell and/or tumour for sufficient time, prior to the introduction of the immune checkpoint blockade agent, to sensitize the cell and/or tumour to the immune checkpoint blockade agent(s). Alternatively, or in addition, the neoplastic cell and/or tumour is exposed to the sensitising agents for sufficient time, prior to the introduction of the immune checkpoint blockade agent, to sensitize the cell and/or tumour to the immune checkpoint blockade agent(s). In one exemplary form of the invention, the sensitising agent is brought in contact with a neoplastic cell and/or tumour that is non-responsive to immune checkpoint agents for at least 3 days prior to immunotherapy. More particularly, the sensitising therapeutic is made to contact with the tumour for between 3 days and 5 weeks at a clinical standard non-toxic dose. In an alternate form of the invention, the sensitising agent is brought in contact with a neoplastic cell and/or tumour that is non-responsive to immune checkpoint agents for such time to activate signal transducer and activator of transcription 1 (STAT1) protein.

In a form of the invention the sensitising agent is brought in contact with a tumour that is non-responsive to immune checkpoint agents for at least 3 days prior to immunotherapy. More particularly, the sensitising therapeutic is made to contact with the tumour for between 3 days and 5 weeks at a clinical standard non-toxic dose. To this end, the therapeutic may be exposed to the tumour for 1, 2, 3, 4 or 5 weeks or any part of a week (such as 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34 or 35 days), prior to immunotherapy. Those skilled in the art will appreciate that the exposure time to the therapeutic will depend on the concentration of the agent used in the patient. Ideally, the sensitising agent will be given to a patient at a non-toxic biologically effective amount (i.e. at an amount that does not harm the patient but is able to sensitize the tumour being treated). Those skilled in the art will know what that does is for each individual agent.

In an alternate form of the invention, a therapeutically effective amount of the sensitising agent is brought in contact with a tumour that is non-responsive to immune checkpoint agents for such time to activate STAT1 protein. Particularly, the inventors have revealed that STAT1 activation is a driver of an inflammatory responsive tumour microenvironment and can serve as a potential biomarker predictive of response to immune checkpoint blockade.

As an alternative measure of sensitisation a tumour or neoplastic cell population will be sensitized when a measurable amount of immune effector cells (such as natural killer cells) is detectable in the tumour or cell population microenvironment.

Preferably, the sensitising agents are administered or contacted with the cell or tumour at a concentration or effective dose that is sufficient to cause a neoplastic cell and/or tumour to be sensitized prior to administration of the immune checkpoint blockade agents. Notably, the amounts of the agent(s) effective for this purpose will vary depending on the type of agent used, as well as the particular factors of each case, including the type of condition, the stage of the condition, the subject's weight, the severity of the subject's condition, and the method of administration. Ideally the concentration of sensitising agent used in the method will be sufficient to activate STAT1 protein in a tumour cell population.

In a preferred form of the first aspect of the invention the method also includes the step of: administering an immunotherapy or an immune checkpoint blockade agent once the one or more checkpoint sensitising agents have attracted sufficient effector immune cells (e.g. IFNγ producing NK cells) to the tumour or neoplastic cell population environment to enhancing the efficacy of the immune therapy or immune checkpoint blockade agent(s) on a malignant condition.

According to the invention, the immune checkpoint blockade agent(s) that is selected for use in the method is an agent that targets the inhibitory T cell molecule CTLA-4 and/or targets the Programmed Death receptor (PD-1) and/or PD-Ligand (PD-L) pathway and/or glucocorticoid-induced tumour necrosis factor receptor (GITR). In a highly preferred form of the invention the blocked immune checkpoint pathway is associated with one or more (a combination) of the following targets: CTLA4, PD1, PD1 ligand/or GITR and/or LAG3 and/or OX40 and/or 41BB and/or TIM3. For example, the immunotherapy is selected from agents that target (a) CTLA-4 such as ipilimumab or tremelimumab. An example of an agent that targets PD-1 is a PD-1 antagonist such as nivolumab, AMP-224, pidilizumab, spartalizumab, cemiplimab, camrelizumab or pembrolizumab. An example of an agent that targets PD-L1 is a PD-L1 antagonist such as Atezolizumab, Avelumab or Durvalumab. Examples of agents that target GITR are antagonists such as TRX518 or MK4166.

To improve on the number of patients who benefit from immune checkpoint blockade, CTLA-4, PD-1, PD-L1 and/or GITR antibodies are combined in a preferred form of the invention. Details of these products, their targets and the cancer types that they are primarily used against are provided in the following Table 1. Examples of agents that target LAG3 are BMS-986016, BI 754111, LAG-525 or REGN-3767. An example of an agent that targets OX40 is BMS 986178, MEDI6469, GSK3174998, PF-04518600. Examples of agents that target TIM3 are LY3321367, MBG453 or TSR-022. An example of an agent that targets 41BB is PF-05082566. In a particularly preferred form of the invention the immune checkpoint blockade agent is one of the agents identified in Table 1, below.

TABLE 1
Antibodies targeting CTLA-4, PD-1 or its
ligand PD-L1 or other immune checkpoints.

| | Target | Company | Cancer Types |
|---|---|---|---|
| ipilimumab | CTLA-4 | BMS | melanoma |
| | | | lung/prostate |
| | | | many other cancers |
| tremelimumab | CTLA-4 | Pfizer | mesothelioma |
| | | | lung/melanoma (with |
| | | | other drugs) |
| nivolumab | PD-1 | BMS | melanoma/lung/kidney |
| | | | many other cancers |
| pembrolizumab | PD-1 | Merck | melanoma/lung |
| | | | many other cancers |
| Atezolizumab | PD-L1 | Roche | melanoma/lung |
| | | | kidney and many other |
| | | | cancers |
| Durvalumab | PD-L1 | AstraZeneca | lung |
| AMP-224 | PD-1 | | melanoma/lung |
| | | | kidney and many other |
| | | | cancers |
| Avelumab | PD-L1 | EMD-Serono | melanoma/lung |
| | | | kidney and many other |
| | | | cancers |
| TRX518 | GITR | GITR Inc. | melanoma |
| Spartalizumab | PD-1 | Novartis | Various cancers |
| Cemiplimab | PD-1 | Sanofi | Various cancers |
| Camrelizumab | PD-1 | Jiangsu HengRui | Various cancers |
| | | Medicine | many other cancers |
| MK4166 | GITR | Merck | melanoma |
| | | | many other cancers |
| BMS-986016 | LAG3 | BMS | Various cancers |
| BI754111 | LAG3 | Boehringer | Various Cancers |
| | | Ingelheim | |
| LAG525 | LAG3 | Novartis | Various cancers |
| REGN-3767 | LAG3 | Regeneron | Various cancers |
| BMS 986178 | OX40 | BMS | Various cancers |
| LY3321367 | TIM3 | Eli Lilly | Various cancers |
| PF-05082566 | 41BB | Pfizer | Various Cancers |
| MBG453 | TIM3 | Novartis | Various Cancers |
| TSR-022 | TIM3 | Tesaro | Various cancers |
| BMS 986178 | OX40 | BMS | Various cancers |
| MEDI6469 | OX40 | MedImmune | Various cancers |
| GSK3174998 | OX40 | GSK | Various cancers |
| PF-04518600 | OX40 | Pfizer | Various cancers |
| PF-04518600 | OX40 | Pfizer | Various cancers |
| Tislelizumab | PD-1 | Beigene | Various cancers |
| SHR1210 | PD-1 | Shanghai Hengrui | Various cancers |
| | | Pharmaceutical Co. | |
| JS-001 | PD-1 | Shanghai Junshi | Various cancers |
| | | Biosciences Co | |
| IBI308 | PD-1 | Innovent Biologics. | Various cancers |

In one embodiment of the first aspect of the invention there is provided a method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase the numbers of NK cells and thereby promote or enhance the sensitivity of the neoplastic cell and/or tumour to an immune check point blockage agent.

Preferably, the NK cells according to any broad aspect, embodiment, form or example of the invention described herein throughout are NK cells which produce IFNγ and/or activated STAT1 protein.

The activated STAT1 protein according to any broad aspect, embodiment, form or example of the invention described herein throughout is typically a phosphorylated STAT1 protein.

In one preferred example, the method causes an increase in the numbers of NK cells at the site of the neoplastic cell and/or tumour and/or at the cellular microenvironment of the neoplastic cell and/or tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In another embodiment of the first aspect of the invention there is provided a method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by the cell and/or tumour thereby promoting or enhancing the sensitivity of the one or more neoplastic cells an immune check point blockage agent.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In yet another embodiment of this first aspect of the invention, there is provided a method for promoting or enhancing the sensitivity of a neoplastic cell and/or neoplastic tumour to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by NK cells thereby promoting or enhancing the sensitivity of the neoplastic cell and/or tumour to an immune check point blockage agent.

In one preferred example, the increased production of IFNγ and/or activated STAT1 protein by NK cells occurs at the site of the neoplastic cell and/or tumour and/or in the microenvironment of the neoplastic cell and/or tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

In an embodiment of the first aspect of the invention there is provided a method for promoting or enhancing the sensitivity of one or more cell populations to immune checkpoint blockade agents, said method comprising the step of:
a. identifying a neoplastic cell population that is resistant to one or more immune checkpoint blockade agents; and
b. administering or exposing the neoplastic cells in the tumour identified in step (a) to a therapeutically effective amount of one or more immune checkpoint sensitising agents, for at least 3 days prior to immunotherapy or until the tumour is at least partially sensitized to an immune checkpoint blockade agent.

In one example, the neoplastic cell is a neoplastic cell in a tumour.

Preferably, the immune checkpoint sensitising agents are selected from: a CD40 agonist, an anti-IL10 antibody, an inducer of interferon alpha/beta signalling, an interferon gamma or a functional variant thereof and/or a retinoid.

More preferably, the immune checkpoint sensitising agents are selected from the group comprising: an agonistic CD40 antibody, anti-IL10, Poly(I:C), a retinoid (such as all-trans retinoic acid) and/or Interferon gamma.

In a preferred form of the method, a combination of immune checkpoint sensitising agents are used in the method, said combination being at least a plurality of the identified sensitising agents selected from the group comprising: a CD40 agonist, an anti-IL10 antibody, an inducer of interferon alpha/beta signalling, and an interferon gamma or a functional variant thereof.

Preferably, the immune checkpoint sensitising agents comprise at least a retinoid, for example tretinoin. In one example, the immune checkpoint sensitising agents comprise at least a retinoid and any one or more of a CD40 agonist and/or an anti-IL10 antibody and/or an inducer of interferon alpha/beta signalling and/or an interferon gamma or a functional variant thereof.

Preferably, the immune checkpoint sensitising agents comprise at least an inducer of interferon alpha/beta signalling such as Poly(I:C). In one example, the immune checkpoint sensitising agents comprise an inducer of interferon alpha/beta signalling such as Poly(I:C), an anti-IL10 antibody and interferon gamma or a functional variant thereof. In another example, the immune checkpoint sensitising agents comprise at least an inducer of interferon alpha/beta signalling such as Poly(I:C), and any one or more of: anti-IL10 antibody and/or interferon gamma or a functional variant thereof and/or a CD40 agonist such as agonistic CD40 antibody. In one such example, the immune checkpoint sensitising agents comprise at least an inducer of interferon alpha/beta signalling such as Poly(I:C), and any one or both of an anti-IL10 antibody and/or interferon gamma or a functional variant thereof. In another example, the immune checkpoint sensitising agents comprise at least a CD40 agonist such as an agonistic anti-CD40 antibody.

Preferably, said combination being at least a plurality of the identified sensitising agents selected from the group comprising: an agonistic CD40 antibody, anti-IL10, Poly(I:C), a retinoid (such as all-trans retinoic acid) and/or Interferon gamma. Ideally, the combination will be a combination of at least three of anti-IL10, Poly(I:C) and interferon gamma or anti-CD40, anti-IL10, a retinoid such as all-trans retinoic acid combination of a STAT1-activating cytokine IFNγ. For example, the combination can be a STAT1-activating cytokine IFNγ, the TLR3 ligand poly(I:C) and an anti-IL-10 antibody.

A tumour will be partially sensitized to an immune check point blockade agent when there is at least a 5% response of the neoplastic cells in the tumour to immune checkpoint blockade with an immune checkpoint blockade agent. More preferably, the response will be a 5, 10, 15, 20, 25, 30, 35, 40, 45, 50, 55, 60, 65, 70, 75, 80, 85, 90, 95, 96, 97, 98 or 99% response of the neoplastic cells in the tumour to immune checkpoint blockade with an immune checkpoint blockade agent. In a particularly preferred for of the invention there is at least a 6, 7, 8, 9, 10, 11, 12, 13, 14, 15, 16, 17, 18, 19, 20, 21, 22, 23, 24, 25 26, 27, 28, 29, 30, 31, 32, 33, 34, 35, 36, 37, 38, 39, 40, 40, 41, 42, 43, 44, 45, 46, 47, 48, 49, 50, 51, 52, 53, 54, 55, 56, 57, 58, 59, 60% response of the tumour to immune checkpoint blockade with an immune checkpoint blockade agent. For example, a tumour will be partially sensitized to an immune check point blockade agent when at least 40% of the tumour responds to immune checkpoint blockade with an immune checkpoint blockade agent.

Preferably, the neoplastic cell population in step (a) of this method is selected by either (i) exposing the cells to one or more immune checkpoint blockade agents and identifying those cells that are resistant to the immune checkpoint blockade agents or (ii) by measuring the activity of STAT1 in a cell population, which cell population may be of tumour or immune origin, wherein the absence of activation of the STAT1 protein (which may be measured by either nuclear STAT1 or phosphorylated STAT1 in a cell population, with a threshold of 50%) presents as biomarker for resistance of that cell population in step (a) to immune checkpoint blockade agents. In one example, a threshold of 50% measure for nuclear STAT1 presents a biomarker for resistance for that cell population in step (a) to immune checkpoint blockade agents. In another example, a threshold of 5% measured for phosphorylated STAT1 presents a biomarker for resistance for that cell population in step (a) to immune checkpoint blockade agents.

As an alternative measure a tumour or neoplastic cell population will be at least partially sensitized to an immune checkpoint blockade agent when a measurable amount of natural killer cells is detected in the tumour microenvironment. Where natural killer cells are used as a measure of sensitivity a natural killer cell count of ˜2% of tumour-infiltrating white blood cells in patients will typically reflect on a change in sensitivity of the tumour to Immune check point blockade agents. More preferably, the natural killer cell count should be at least 2, 3, 4, 5, 6, 7, 8, 9, 10, 11, 12, 13, 14, 15, 16, 17, 18, 19 or 20% of tumour-infiltrating white blood cells in patients. Desirably, the natural killer cell count of tumour-infiltrating white blood cells of at least 5 to 10% will typically reflect on a change in sensitivity of the tumour to Immune check point blockade agents.

In an alternate form of the invention a measurable amount of natural killer cells can be reflected by an increase in such cells relative to pre-treatment levels. Where such measurements are made relative to the pre-treatment levels then a relative increase of natural killer cells by about 35, 40, 45, 50, 55, 60, 65% compared to pre-treatment levels of natural killer cells in the tumour will typically reflect on a change in sensitivity of the tumour to Immune check point blockade agents.

In another form of this method, the cells of step (b) will have been exposed to an immune checkpoint blockade agent for a sufficient period of time when measurable amounts of the STAT1 biomarker is detected in the cell population. Such measurable amounts are preferably at least a 40% response, but more preferably at least 50, 55, 60, 65, 70, 75, 80, 85, 90, 95% response in a nuclear STAT1 test and/or at least 5% response in phosphorylated STAT1 test, as herein described.

In this preferred form of the invention, the cell population in step (b) is measured on a periodic basis (optionally, hourly or every 2, 3, 4, 5, 6, 7, 8, 9, 10, 11, 12, 13, 14 , 15, 16, 17, 18, 19, 20, 21, 22, 23 24, 36, 48 hours) for activation of STAT1, wherein the activation and/or presence of the biomarker STAT1 is indicative of cell sensitivity to one or more immune checkpoint blockade agents.

In another embodiment of the first aspect of the invention there is provided a use of one or more immune checkpoint sensitising agents, for promoting or enhancing the sensitivity of a tumour microenvironment to immune checkpoint blockade agents wherein the sensitising agent(s) is administered at least 3 days prior to immunotherapy at a therapeutically effective amount to a tumour that is resistant to one or more immune checkpoint blockade agents.

In a preferred form of the invention the method also includes the step of administering an immunotherapy or an immune checkpoint blockade agent once the one or more checkpoint sensitising agents have attracted sufficient effector immune cells (e.g. IFNγ and/or activated STAT1 producing NK cells) to the tumour or neoplastic cell population environment for enhancing the efficacy of the immune therapy or immune checkpoint blockade agent(s) on a malignant condition.

In another preferred form, the method includes the step of administering an immunotherapy or an immune checkpoint blockade agent once the one or more checkpoint sensitising agents have resulted in sufficient increase in the amount of the immune effectors IFNγ and/or activated STAT1 at the tumour or neoplastic cell population environment for enhancing the efficacy of the immune therapy or immune checkpoint blockade agent(s) on a malignant condition. For example, the IFNγ and/or activated STAT1 is produced by NK cells and/or by the tumour or neoplastic cell population.

According to the invention, the immune checkpoint blockade agent(s) that is selected for use in the method is an agent that targets the inhibitory T cell molecule CTLA-4 and/or targets the Programmed Death receptor (PD-1) and/or or PD-Ligand (PD-L) pathway and/or and/or glucocorticoid-induced tumour necrosis factor receptor (GITR) and/or Lymphocyte-activation gene (LAG)3 tumor necrosis factor receptor superfamily, member 4, also known as CD134 and/or OX40 and/or 41BB and/or t-cell immunoglobulin and mucin-domain containing-(TIM)3. An example of an agent that targets the inhibitory T cell molecule CTLA-4 is a CTLA-4 antagonist such as ipilimumab or tremelimumab. An example of an agent that targets PD-1 is a PD-1 antagonist such as nivolumab, AMP-224, pidilizumab, spartalizumab, cemiplimab, camrelizumab, tislelizumab or pembrolizumab. An example of an agent that targets PD-L1 is a PD-L1 antagonist such as Atezolizumab, Avelumab or Durvalumab. Examples of agents that target GITR are antagonists such as TRX518 or MK4166. Examples of agents that target LAG3 are BMS-986016, BI 754111, LAG-525 or REGN-3767. An example of an agent that targets OX40 is BMS 986178, MED16469, GSK3174998, PF-04518600. Examples of agents that target TIM3 are LY3321367, MBG453 or TSR-022. An example of an agent that targets 41BB is PF-05082566.

According to a second aspect, the invention resides in the use of a therapeutically effective amount of one or more immune checkpoint sensitising agents, in the manufacture of a medicament for sensitising a tumour wherein said tumour is resistant to an immune checkpoint blockade agent.

In a related embodiment the invention resides in the use of a therapeutically effective amount of one or more immune checkpoint sensitising agents, in the manufacture of a medicament for sensitising a tumour wherein said tumour is resistant to an immune checkpoint blockade agent, wherein said medicament increases the numbers of NK cells (such as NK cells producing activated STAT1- and/or IFNγ) and/or increases IFNγ and/or activated STAT1 production by neoplastic cells and/or tumour cells and/or NK cells.

Preferably, the medicament includes instructions to administered to a tumour that is resistant to one or more immune checkpoint blockade agents the immune checkpoint sensitising agents at least 3 days prior to an immunotherapy.

Preferably, the immune checkpoint sensitising agents are selected from: a CD40 agonist, an anti-IL10 antibody, an inducer of interferon alpha/beta signalling, an interferon gamma or a functional variant thereof and/or a retinoid. More preferably, the immune checkpoint sensitising agents are selected from the group comprising: an agonistic CD40 antibody, anti-IL10, Poly(I:C), a retinoid (such as all-trans retinoic acid) and/or Interferon gamma. In a particularly preferred form, a combination of immune checkpoint sensitising agents are used in the method, said combination being at least a plurality of the identified sensitising agents selected from the group comprising: an agonistic CD40 antibody, anti-IL10, Poly(I:C), a retinoid (such as all-trans retinoic acid) and/or Interferon gamma. Ideally, the combination will be a combination of at least three of an anti-IL10, Poly(I:C) and interferon gamma or anti-CD40, anti-IL10, a retinoid such as all-trans retinoic acid or a STAT1-activating cytokine IFNγ. For example, the combination can be a STAT1-activating cytokine IFNγ, the TLR3 ligand poly(I:C) and an anti-IL-10 antibody.

According to a third aspect, the invention resides in a method for treating a patient with either (1) a malignant condition or (2) a post-operative surgical resection of cancer or (3) in advance of, during or following any other form of adjuvant immunotherapy, said method comprising the step of:
a. identifying a tumour or neoplastic cell population(s) that is resistant to one or more immune checkpoint blockade agents; and
b. administering or exposing the tumour or neoplastic cell population(s) identified in step (a) to a therapeutically effective amount of one or more immune checkpoint sensitising agents, for at least 3 days prior to immunotherapy until the tumour is at least partially sensitized to an immune checkpoint blockade agent.

In a preferred form of the third aspect of the invention the method also includes the step of administering an immunotherapy or an immune checkpoint blockade agent once the one or more checkpoint sensitising agents have attracted sufficient effector immune cells (e.g. IFNγ and/or STAT1 producing NK cells) to the tumour or neoplastic cell population environment to enhancing the efficacy of the immune therapy or immune checkpoint blockade agent(s) on a malignant condition.

Preferably, the tumour cell population in step (a) of this method is identified by either (i) exposing the cells to one or more immune checkpoint blockade agents and determining if those cells are resistant to the immune checkpoint blockade agents or (ii) by measuring the activity of STAT 1 in a cell population which cell population may be of tumour or immune origin wherein the absence of 50% activation of the STAT1 protein (for example by nuclear STAT1 test or phosphorylated STAT1 test) presents as a biomarker of resistance of the cell population of step (a) to immune checkpoint blockade agents. In another example, the absence of 5% phosphorylation of STAT1 protein (phosphorylated STAT1 test) presents as a biomarker of resistance of the cell population of step (a) to immune checkpoint blockade agents.

In another preferred form of this method, said method includes the additional step of exposing the cells of step (b) to an immune checkpoint blockade agent when (i) at least 50% activation of the STAT1 protein (for example by nuclear STAT1 test or phosphorylated STAT1 test) is detected in the cell population, and/or (ii) a measurable amount of natural killer cells is detected in the tumour microenvironment.

In another preferred form of this method, said method includes the additional step of exposing the cells of step (b) to an immune checkpoint blockade agent when (i) at least 5% activation of the STAT1 protein (measure by phosphorylated STAT1 test) is detected in the cell population, and/or (ii) a measurable amount of natural killer cells is detected in the tumour microenvironment.

In the method of the present invention, an amount of sensitising therapeutic agent that is administered to the patient will be that amount that is sufficient to sensitise the patient's neoplastic cells to immune checkpoint agents. Notably, the amounts of the agent(s) effective for this purpose will vary depending on the type of agent used, as well as the particular factors of each case, including the type of condition, the stage of the condition, the subject's weight, the severity of the subject's condition, and the method of administration. These amounts can be readily determined by the skilled artisan.

In an embodiment of the third aspect of the invention there is provided a use of one or more sensitising therapeutic agents selected from an agonistic CD40 antibody, anti-IL10, TLR3 ligand Poly(I:C) , a retinoid (such as all-trans retinoic acid) and/or Interferon gamma, for promoting or enhancing, in a patient, the sensitivity of a tumour to immune checkpoint blockade agents wherein the sensitising therapeutic agent is administered to the tumour that is resistant to one or more immune checkpoint blockade agents at least 3 days prior to immunotherapy.

In another preferred form of this method, said method includes the additional step of exposing the cells of step (b) to an immune checkpoint blockade agent when measurable amounts of (i) the activated STAT1 and/or IFNγ are detected in the cell population and/or (ii) measurable amount of natural killer cells (such as NK cells producing activated STAT1 and/or IFNγ) are detected in the tumour or cell population cellular microenvironment.

In an embodiment of the third aspect of the invention there is provided a use of one or more immune checkpoint sensitising agents, for promoting or enhancing, in a patient, the sensitivity of a tumour to immune checkpoint blockade agents wherein the sensitising agent(s) is/are administered to the tumour that is resistant to one or more immune checkpoint blockade agents at least 3 days prior to immunotherapy. In one example, the one of more sensitising agent(s) may then be further administered concurrently with the administration of the one or more immune checkpoint blockade agents. For example, the one of more sensitising agent(s) may then be further administered concurrently with the administration of the one or more immune checkpoint blockade agents for the duration of the ICB therapy, for example up to at least 3 months, or at least, 10 months or at least 9 months, or at least 12 months or at least 13 months or at least 14 months or at least 15 months or at least 16 months or at least 17 months or at least 18 months or at least 19 months or at least 20 months or at least 21 months or at least 22 months or at least 23 months or at least 24 months or at more than two years.

According to a fourth aspect, the invention resides in a method of treating a patient with a tumour or neoplastic cell population comprising the step of: treating the tumour or neoplastic cell population with combination of a therapeutically effective amount of a STAT1-activating cytokine IFNγ, a TLR3 ligand poly(I:C) and an anti-IL-10 antibody for sufficient time prior to immunotherapy to attract immune cells and in particular IFNγ producing NK cells into the tumour, sensitizing the tumors to immune checkpoint blockade. In a form of this aspect of the invention, the combination is brought in contact with a tumour for at least 3 days prior to immunotherapy. More particularly, the combination is made to contact with the tumour for between 3 days and 5 weeks at a clinical standard non-toxic dose prior to immunotherapy.

According to a fifth aspect, the invention resides in a sensitising therapeutic comprising at least one immune checkpoint sensitising agent(s), for enhancing the efficacy of immune checkpoint blockade agents on a malignant condition. Preferably, the sensitising composition is a combination of at least a plurality of the identified agents. Ideally, the combination will be a combination of at least three of anti-IL10, poly(I:C) and interferon gamma or anti-CD40, anti-IL10, or a retinoid such as all-trans retinoic acid and interferon gamma. For example, the combination can be a STAT1-activating cytokine IFNγ, the TLR3 ligand poly(I:C) and an anti-IL-10 antibody. With each composition there may be included an immunotherapeutic agent that can be administered after the sensitising therapeutic composition once the effect of the sensitising therapeutic composition has had effect.

According to the invention there is also provided a sensitising therapeutic comprising:
a) a therapeutically effective amount of one or more immune checkpoint sensitising agents, and
b) a pharmaceutically acceptable carrier.

The sensitising therapeutic according to the invention can comprise one or more of a CD40 agonist, an anti-IL10 antibody, an inducer of interferon alpha/beta signalling, an interferon gamma or a functional variant thereof and/or a retinoid. In one example, the sensitising therapeutic can comprise an agonistic CD40 antibody, anti-IL10, TLR3 ligand Poly(I:C), a retinoid such as all-trans retinoic acid and/or Interferon gamma. For example, the sensitising therapeutic may be provided as a monotherapy. Preferably, the sensitising therapeutic is provided as a combination of therapeutics which together work to exert their biological effect of sensitizing tumour cells.

Therapeutics of the invention can be combined with various components to produce compositions of the invention. Such compositions can comprise, for example, one or more of the identified therapeutics in a therapeutically effective amount of the compound in admixture with a pharmaceutically or physiologically acceptable formulation agent selected for suitability with the mode of administration.

Medicaments of the invention can also be combined with a pharmaceutically acceptable carrier or diluent to produce a pharmaceutical composition. Suitable carriers and diluents include isotonic saline solutions, for example phosphate-buffered saline. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. See, for example, Remington's Pharmaceutical Sciences, 19th Ed. (1995, Mack Publishing Co., Easton, Pa.) which is herein incorporated by reference.

A pharmaceutical composition can also contain formulation materials for modifying, maintaining or preserving, for example, the pH, osmolarity, viscosity, clarity, colour, isotonicity, odour, sterility, stability, rate of dissolution or release, adsorption or penetration of the composition. Suitable materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin), fillers; monosaccharides, disaccharides; and other carbohydrates (such as glucose, mannose, or dextrins); proteins (such as serum albumin, gelatin or immunoglobulins); colouring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapol); stability enhancing agents (sucrose or sorbitol); tonicity enhancing agents (such as alkali metal halides, preferably sodium or potassium chloride), delivery vehicles, diluents, excipients and/or pharmaceutical adjuvants.

The optimal concentration of the therapeutic use in a composition will be determined by one skilled in the art depending upon, for example, the intended route of administration, delivery format, and desired dosage.

Such compositions can influence the physical state, stability, rate of in vivo release, and rate of in vivo clearance of the peptide of the invention. Moreover, the preferred form of the pharmaceutical composition depends on the intended mode of administration and therapeutic application.

According to an embodiment of the invention, the invention resides in therapeutic composition selected from an agonistic CD40 antibody, anti-IL10, Poly-I:C, a retinoid (such as all-trans retinoic acid) and/or Interferon gamma that enhances the response that immune checkpoint blockade agents have on a malignant condition.

The administration of the agents in the therapeutic combination may occur concurrently, sequentially, or alternately. Concurrent administration refers to administration of the sensitising therapeutic agent and the immune checkpoint blockade agent at essentially the same time. For concurrent co-administration, the courses of treatment may also be run simultaneously. For example, a single, combined formulation of the agents may be administered to the patient.

In one example, the one of more sensitising agent(s) may then be further administered concurrently with the administration of the one or more immune checkpoint blockade agents. For example, the one of more sensitising agent(s) may then be further administered concurrently with the administration of the one or more immune checkpoint blockade agents for the duration of the ICB therapy, for example up to at least 3 months, or at least 6 months, or at least 9 months, or at least 10 months, or at least 11 months, or at least 12 months or at least 13 months or at least 14 months or at least 15 months or at least 16 months or at least 17 months or at least 18 months or at least 19 months or at least 20 months or at least 21 months or at least 22 months or at least 23 months or at least 24 months or more than two years.

The compositions of the invention may be presented in unit or multi-dose containers, such as sealed ampoules or vials.

Additional pharmaceutical compositions will be evident to those skilled in the art, including formulations involving a peptide of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT Application No. PCT/US93/00829 that describes the controlled release of porous polymeric microparticles for the delivery of pharmaceutical compositions. Additional examples of sustained-sustained-release preparations include semipermeable polymer matrices in the form of shaped articles, for example, films, or microcapsules. Sustained release matrices may include polyesters, hydrogels, polylactides, copolymers of L-glutamic acid and gamma ethyl-L-glutamate, ethylene vinyl acetate or poly-D(−)-3-hydroxybutyric acid. Sustained-release compositions may also include liposomes, which can be prepared by any of several methods known in the art. Such compositions can provide a means for delivering one therapeutic followed by the release of a second. To illustrate this the therapeutic agent of the invention can be delivered first to a patient with a time delay followed by an immunotherapy

The effective amount of the therapeutic in the therapeutic composition to be employed therapeutically will depend, for example, upon the therapeutic context and objectives. One skilled in the art will appreciate that the appropriate dosage levels for treatment will thus vary depending, in part, upon the molecule delivered, the indication for which the therapeutic is being used, the route of administration, and the size (body weight, body surface or organ size) and condition (the age and general health) of the patient. Accordingly, the clinician may titre the dosage and modify the route of administration to obtain the optimal therapeutic effect. A typical dosage may range from about 0.1 μg/kg to up to about 100 mg/kg or more, depending on the factors mentioned above. In other embodiments, the dosage may range from 0.1 μg/kg up to about 100 mg/kg; or 1 μg/kg up to about 100 mg/kg; or 5 μg/kg up to about 100 mg/kg.

The frequency of dosing will depend upon the pharmacokinetic parameters of the therapeutic and the formulation used. Typically, a clinician will administer the therapeutic until a dosage is reached that achieves the desired effect. The therapeutic may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g., orally, through injection by intravenous, intracoronary, intraperitoneal, intracerebral (intra-parenchymal), intracerebroventricular, intramuscular, intra-ocular, intraarterial, intraportal, intralesional, intra-tumoural or intralesional routes; by sustained release systems or by implants. Where desired, the compositions may be administered by bolus injection or continuously by infusion, or by implantation device.

Preferably, compositions of the invention are delivered by injection, including, without limitation, intralesional, intra-tumoural, epifascial, intracapsular, intracutaneous, intramuscular, intraorbital, intraperitoneal (particularly in the case of localized regional therapies), intraspinal, intrasternal, intravascular, intravenous, parenchymatous, intra-tumoural or subcutaneous.

When parenteral administration is contemplated, the therapeutic for use in this invention may be in the form of a pyrogen-free, parenterally acceptable aqueous solution comprising the desired peptide of the invention in a pharmaceutically acceptable vehicle. A particularly suitable vehicle for parenteral injection is sterile distilled water in which the active agent is formulated as a sterile, isotonic solution, properly preserved. Yet another preparation can involve the formulation of the desired molecule with an agent, such as injectable microspheres, bio-erodible particles, polymeric compounds (such as polylactic acid, acid or polyglycolic acid), or beads or liposomes, that provides for the controlled or sustained release of the product which may then be delivered as a depot injection. Hyaluronic acid may also be used, and this may have the effect of promoting sustained duration in the circulation. Other suitable means for the introduction of the desired molecule include implantable drug delivery devices.

In one embodiment, a therapeutic may be formulated for inhalation. For example, a peptide may be formulated as a dry powder for inhalation. The therapeutic inhalation solution may also be formulated with a propellant for aerosol delivery. In yet another embodiment, solutions may be nebulized.

It is also contemplated that certain therapeutic may be administered orally. For example, the therapeutic can be designed to release the active portion of the formulation at the point in the gastrointestinal tract when bioavailability is maximized and pre-systemic degradation is minimized. Additional agents can be included to facilitate absorption of the active agent. Diluents, flavourings, low melting point waxes, vegetable oils, lubricants, suspending agents, tablet disintegrating agents, and binders may also be employed. Alternatively, the therapeutic can be prepared with non-toxic excipients in tablet form.

Alternatively, or additionally, the composition may be administered locally via implantation of a membrane, sponge or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use the pharmaceutical compositions herein in an ex vivo manner. In such instances, cells, tissues, or organs that have been removed from the subject to be treated are exposed to the therapeutic after which the cells, tissues and/or organs are subsequently implanted back into the subject.

According to the invention there is also provided a therapeutic composition for use in sensitizing a tumour to immune checkpoint blockade agents, comprising:
a. a therapeutically effective amount of one or more of an agonistic CD40 antibody, anti-IL10, TLR3 ligand Poly-I:C, a retinoid (such as all-trans retinoic acid) and/or Interferon gamma, and
b. a pharmaceutical acceptable carrier.

Preferably, the therapeutic composition for use in sensitizing a tumour to immune checkpoint blockade agents, will consist essentially of a therapeutically effective amount of a combination of at least a plurality of the identified agents. Ideally, the composition will consist of a therapeutically effective amount of at least a combination of anti-IL10, poly-I:C and interferon gamma or anti-CD40, anti-IL10 and interferon gamma.

The administration of the agents in the therapeutic combination may occur concurrently, sequentially, or alternately. Concurrent administration refers to administration of the therapeutic agent and the immune checkpoint blockade agent at essentially the same time. For concurrent co-administration, the courses of treatment may also be run simultaneously. For example, a single, combined formulation of the agents may be administered to the patient.

In one example, the one of more sensitising agent(s) may then be further administered concurrently with the administration of the one or more immune checkpoint blockade agents. For example, the one of more sensitising agent(s) may then be further administered concurrently with the administration of the one or more immune checkpoint blockade agents for the duration of the ICB therapy, for example up to at least 3 months, or at least 6 months, or at least 9 months, or at least 10 months, or at least 11 months, or at least 12 months or at least 13 months or at least 14 months or at least 15 months or at least 16 months or at least 17 months or at least 18 months or at least 19 months or at least 20 months or at least 21 months or at least 22 months or at least 23 months or at least 24 months or more than two years

According to a sixth aspect, the invention resides in a kit for treating a tumour or a population of neoplastic cells the kit comprising:
a) a therapeutically effective amount of one or more immune checkpoint sensitising agents, and
b) instructions to administer the immune checkpoint sensitising agents to a tumour that is resistant to one or more immune checkpoint blockade agents at least 3 days prior to an immunotherapy.

Preferably, the kit also includes one or more immune checkpoint blockade agents and/or immunotherapeutic agents, and instructions to administer the agent or agents to the tumour or neoplastic cell population once the one or more checkpoint sensitising agents have attracted sufficient effector immune cells (e.g. IFNγ producing NK cells) to the tumour or neoplastic cell population environment. The effect of this is to cause tumour cell sensitisation, enhancing the efficacy of immune checkpoint blockade agents on a malignant condition.

According to a seventh aspect, the invention resides a diagnostic method for predicting a response to immune checkpoint blockade comprising the steps of:
a. measuring STAT1 activation in a tumour; and
b. determining whether the tumour is resistant to immune checkpoint blockade agents wherein the activation and/or localisation of the biomarker STAT1 is indicative of the cells developing sensitivity to one or more immune checkpoint blockade agents.

Desirably, STAT1 is measured by a nuclear STAT1 test or a phosphorylated STAT1 test is used. In these tests a measure of at least 50% positive staining or identification of phosphorylated STAT1 is sufficient as an indicator of sensitization of the tumour cell population to immunotherapy with immune checkpoint blockade agents.

According to an eighth aspect, the invention resides a diagnostic method for predicting a response to immune checkpoint blockade comprising the steps of:
a. measuring natural killer cell presence in a tumour; and
b. determining whether the tumour is resistant to immune checkpoint blockade agents wherein the activation and/or presence of natural killer cells is indicative of the cells developing sensitivity to one or more immune checkpoint blockade agents.

Where natural killer cells are used as a measure for sensitivity a natural killer cell count of ˜2% of tumour-infiltrating white blood cells in patients will typically reflect on a change in sensitivity of the tumour to Immune check point blockade agents. More preferably, the natural killer cell count should be at least 2, 3, 4, 5, 6, 7, 8, 9, 10, 11, 12, 13, 14, 15, 16, 17, 18, 19 or 20% of tumour-infiltrating white blood cells in patients will typically reflect on a change in sensitivity of the tumour to Immune check point blockade agents. Desirably, the natural killer cell count of tumour-infiltrating white blood cells of at least 5 to 10% will typically reflect on a change in sensitivity of the tumour to Immune check point blockade agents.

In an alternate form of the invention a measurable amount of natural killer cells can be reflected by an increase in such cells relative to pre-treatment levels. Where such measurements are made relative to the pre-treatment levels then a relative increase of natural killer cells by about 35, 40, 45, 50, 55, 60, 65% compared to pre-treatment levels of natural killer cells in the tumour will typically reflect on a change in sensitivity of the tumour to Immune check point blockade agents.

According to a further aspect, the invention resides a diagnostic method for predicting a response to immune checkpoint blockade comprising the steps of:
a. measuring STAT1 activation and/or IFNγ production in a cell population; and
b. determining whether the tumour is resistant to immune checkpoint blockade agents wherein the activation and/or presence of the biomarker STAT1 is indicative of the tumour cells developing sensitivity to one or more immune checkpoint blockade agents.

Preferably, measuring STAT1 activation and/or IFNγ production comprises measuring STAT1 activation and/or IFNγ and/or a neoplastic cell population and/or a tumour.

According to a ninth aspect of the invention, there is provided a method for immobilising NK cells or increasing the number of NK cells at site of a neoplastic cell and/or tumour in a subject and/or to the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

Preferably, the NK cells produce IFNγ and/or activated STAT1 protein.

According to a tenth aspect of invention, there is provided a method of inducing or increasing production of IFNγ and/or activated STAT1 protein by NK cells in a subject, for example at a site of the neoplastic cell and/or tumour in the subject and/or in the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

In an eleventh aspect of the invention, there is provided a method of inducing or increasing production of IFNγ and/or activated STAT1 protein by a neoplastic cell and/or tumour in a subject, said method comprising administering to the subject one or more sensitising agents selected from a CD40 agonist, an anti-IL10 antibody, prior to treatment of the subject with one or more immune check point blockade agents.

Preferably, the one or more sensitising agents is/are administered to the subject at the site of the neoplastic cell and/or tumour or at the cellular microenvironment of the neoplastic cell and/or tumour.

In one example, the method comprises administering to the one or more sensitising agents to a neoplastic cell and/or tumour in a subject or exposing a neoplastic cell and/or tumour in the subject to the one or more sensitising agents prior to treatment with the one or more immune checkpoint blockade agents.

In one example according to any broad aspect, embodiment, form or example of the invention described herein the immune checkpoint sensitising agents are selected from: a CD40 agonist, an anti-IL10 antibody, an inducer of interferon alpha/beta signalling, an interferon gamma or a functional variant thereof and/or a retinoid.

In one example, the neoplastic tumour according to any aspect, embodiment, form or example of the invention as described herein throughout is a malignant or tumour or benign tumour.

In one example, the neoplastic cell or cell population according to any aspect, embodiment, form or example of the invention as described herein throughout is malignant or benign.

In one preferred example, the neoplastic cell or cell population comprise one or more cancer tumour cells selected from melanoma tumours, non-small cell lung cancer tumours, Merkel-cell carcinoma tumours, microsatellite instable colorectal cancer tumours, renal cancer tumours, and/or mesothelioma cancer tumours.

### EXAMPLES

The following examples serve to describe the present invention further and should not be construed as limiting.

### Example 1: Study Design

The studies shown in the examples that follow seek to define the neoplasic cellular microenvironment before treatment with immune check point blockade agent(s) associated with an effective outcome by comparing gene expression and flow cytometry data from ICB responsive and non-responsive tumours within the same mouse cancer model.

The studies shown in the examples that follow also seek to demonstrate a method for therapeutically promoting or enhancing the tumour microenvironment towards a responsive phenotype i.e., a phenotype which is susceptible to treatment with ICB agents, and thus increase the response to ICB.

By implanting a tumour subcutaneous (s.c.) on both flanks of a mouse, it was possible to remove one entire tumour 1 hour prior to therapy with ICB, then monitor the remaining tumour for response to ICB. The removed tumour was analysed by flow cytometry, bulk whole transcriptome shotgun sequencing (bulk RNAseq) or single cell whole transcriptome shotgun sequencing (single cell RNAseq), and categorised as a responsive or non-responsive tumour based on the outcome of the corresponding tumour left in situ. Tumours that showed an intermediate response (partial response or relapse) were excluded from analysis. These experiments were performed using AB1 mesothelioma and Renca renal cell carcinoma cell lines.

In order to exclude mouse model-specific effects, genes that were differentially expressed between responsive and non-responsive tumours in both models were focused on. The sample size for the bulk RNAseq experiments was estimated using the method developed by Hart et al (as detailed S. N. Hart, et al., (2013) Calculating sample size estimates for RNA sequencing data. J Comput Biol 20, 970-978).

The transcriptomic data was then used to identify pathways and regulators that could be targeted. The findings were validated using publicly available RNAseq data from two cancer patient cohorts (N. Riaz et al., (2017) Cell 171, 934-949 e915; and S. Mariathasan et al., (2018) Nature 554, 544-548). In vivo targeting studies were utilised with tumour growth as an endpoint.

These studies were performed in BALB/c mice carrying AB1 and Renca tumours, and were validated in unrelated s.c AE17 mesothelioma and B16 melanoma models in C57BL/6 mice, to establish robustness across tumour models and mouse strains. Mice were randomized after tumours were established, prior to tumour removal and therapy. Treatments were administered by one researcher, while tumours were measured by another researcher who was blinded for treatment allocation. Detailed sample size calculations and statistical analyses employed are outlined in example 2 below.

### Example 2: Methods and Materials

### 1. Mice

BALB/cArc, BALB/cJAusb and C57BL6/J mice 8-12 weeks of age were used for all experiments. Mice were obtained from the Animal Resource Centre (Murdoch, Wash., Australia), or the Australian BioResources (Moss Vale, NSW) and housed at the Harry Perkins Institute of Medical Research Bioresources Facility under specific pathogen free conditions. Mice were fed Rat and Mouse cubes (Specialty Feeds, Glen Forrest, Australia) and had access to water ad libitum. Cages (Techniplast, Italy) were individually ventilated with filtered air, contained aspen chips bedding (Tapvei, Estonia) and were supplemented with tissues, cardboard rolls and wood blocks as environmental enrichment. Cages and were changed every 14 days. Mice were housed at 21-22° C. with 12-hour light/dark cycle (06:00-18:00). Sentinel mice (n=3) in the animal facility were screened monthly for a standard panel of bacteria and fungi, ectoparasites, endoparasites, non-pathogenic protozoa and viruses (Cerberus Sciences). All experiments were conducted in compliance with the institutional guidelines provided by the Harry Perkins Institute for Medical Research animal ethics committee (approval numbers AE07, AE047, AE091).

### 2. Cell Culture

Mouse mesothelioma cell lines AB1 and AE17 were obtained from Cell Bank Australia. The mouse renal cortical adenocarcinoma cell line Renca was kindly donated by Dr E. Sotomayor and Dr F. Cheng (University of South Florida, Tampa, Fla.) and can also be obtained from ATCC (Manassas, Va.; CRL-2947). The murine melanoma cell line B16 was obtained from ATCC (Manassas, Va.; CRL-6475). Cell lines were maintained in RPMI 1640 (Invitrogen, Mulgrave, Australia) supplemented with 20 mM HEPES, 0.05 mM 2-mercaptoethanol, 100 units/ml penicillin (CSL, Melbourne, Australia), 50 μg/ml gentamicin (David Bull Labs, Kewdale, Australia), and 10% FCS (Invitrogen). Cells were grown to 70-80% before passage and passaged between 3-5 times before inoculation. Cells were frequently tested for mycoplasma by PCR and remained negative. Cell lines were validated yearly by flow cytometry for MHC class I molecules H2-K^{b} (consistent with C57BL/6) and H2-K^{d} (consistent with BALB/c), and for fibroblast markers E-cadherin (E-cad), Epithelial cell adhesion molecule (EpCam) and platelet-derived growth factor receptor α (PDGFRα) (negative) and by PCR for mesothelin (positive for AB1, negative for Renca).

### 3. In Vivo Treatments

When cell lines were 70-80% confluent, they were harvested and washed 3 times in PBS. 5×10⁵ cells in 100 μl was inoculated subcutaneously (s.c.) onto the lower right hand side (RHS) flank (for single tumour inoculations) or both flanks (for dual tumour inoculations) using a single 26G needle per injection. Mice were randomised when tumours became palpable, approximately 3-5 days after tumour inoculation. Tumours were measured at least 3 times weekly using calipers by a researcher who was blinded for treatment allocation to guarantee blinded assessment of the primary endpoint.

### 4. Surgery Experiments

Seven (AB1) or 10 (Renca) days post tumour inoculation, when tumours were ˜9 mm², mice were dosed with 0.1 mg/kg buprenorphine in 100 μl s.c. (30 min prior) and anesthetised using isoflurane (4% in 100% oxygen at a flow rate of 2 L/min). Whole tumours and the corresponding draining inguinal lymph node on the RHS were removed by surgical excision and immediately immersed in RNAlater (Life Technologies, Australia) for RNAseq, or cold PBS for single cell RNAseq or flow cytometry. The wound was closed with staples (Able Scientific, Australia). Mice were placed in a heat box for recovery. One hour after surgery, mice were administered immune checkpoint blockade (ICB). The remaining tumour was monitored for response as an indicator of response for the removed tumour. Mice were designated as “responders” when their tumour completely regressed, and they remained tumour free for up to 4 weeks after treatment. Mice were designated as “non-responders” if their tumours grew out to 100 mm² within 4 weeks after start of treatment, similar to saline-treated controls. Mice that had a delay in tumour growth or partial regression were designated as ‘intermediate responders’ and were excluded from the analysis. For internal consistency, experiments were only performed in which mice displayed a dichotomous response; i.e., in any cage there had to be at least one non-responder amongst responders or vice versa.

### 5. In Vivo Immune Checkpoint Blockade (ICB) Treatment

The anti-PD-L1 hybridoma (clone MIH5) (Bioceros, The Netherlands) and the anti-CTLA4 hybridoma (clone 9H10) ((Bioceros, The Netherlands) were cultured in IMDM containing 1% of FCS and gentamycin. Clarified supernatants were used to purify the antibody using affinity chromatography. The antibody was sterile formulated in PBS. Mice received an intraperitoneal (i.p.) dose of 100 μg of anti-CTLA4 and 100 μg anti-PDL1 combined in 100 μl phosphate-buffered solution (PBS). Mice received additional doses of 100 μg anti-PDL1 two and four days later. Vehicle controls received PBS alone. In previous experiments (W. J. Lesterhuis et al., (2013) PLoS One 8, e61895) by the inventors did not find any difference in effect of control IgG versus PBS, and therefore vehicle controls received PBS alone. The time of treatment initiation was varied after tumour inoculation so as to obtain a suitable background response rates to ICB; high for experiments in which responses were to be negated, and low in experiments in which the response rate was to be improved.

### 6. Tumour Preparation for RNA Sequencing

Bulk RNAseq, flow cytometry and single cell RNAseq were performed on tumours from separate experiments.

For RNAseq, whole tumours and lymph nodes were surgically reselected, the surrounding tissue was removed and immediately submerged in RNAlater (Life Technologies, Australia). Samples were stored at 4° C. for 24 hours, after which supernatant was removed and samples transferred to −80° C.

Frozen tumours were dissociated in Trizol (Life Technologies, Australia) using a TissueRuptor (QIAgen, Australia). RNA was extracted using chloroform and purified on RNeasy MinElute columns (QIAgen, Australia). The integrity of the RNA samples was confirmed on the Bioanalzyer (Agilent Technologies, USA). Library preparation and sequencing (50 bp, single-end) was performed by Australian Genome Research Facility, using Illumina HiSeq standard protocols.

### 7. Single Cell RNAseq

For single cell RNA seq, tumours were harvested, and submerged in cold PBS, cut into 1-2 mm pieces with a scalpel blade and dissociated using the GentleMACS system (Miltenyi Biotec, Germany) until processed for single cell profiling. Cryo-stored cells were rapidly thawed and diluted in PBS and pelleted. Pellets were resuspended in PBS and passed through a 40 μM filter to remove cell clumps. Approximately 5,000 cells per sample were then loaded onto a 10x genomics Chromium controller to generate Chromium Single Cell 3′ Libraries. Sequencing was carried out by the Australian Genome Research Foundation in Melbourne Australia.

Primary analysis was carried out using the 10× genomics cell ranger software suite. Raw sequencing data was de-multiplexed (cellranger v2.1.1 and bcl2fastq v2.20.0.422), reads aligned to the reference (GRCm38, Ensembl 84 build) and gene expression quantified using the cell ranger count command. Using default parameters some droplets containing real cells were discarded. Therefore the expected-cells 6000 flag was used to include these cells. Responder and non-responder samples were combined using cellranger aggr function. All cells in the samples were annotated using the SingleR software package as described in D. Aran et al., (2018) BioRxn, 284604.

### 8. Flow Cytometry

For flow cytometry, tumours were harvested and immediately submerged in cold PBS, cut into 1-2 mm pieces with a scalpel blade and dissociated using the GentleMACS system. Fc block (anti-CD16/CD32, BD) was used for 10 minutes on ice. UV Zombie live/dead (Biolegend) was used to discriminate live cells. Cells were permeabilized and fixed using FoxP3 fix/Perm buffer kit (Biolegend) before addition of antibodies for 20 minutes at RT. See Supplementary Table 2 for antibody details. Cells were kept in stabilizing fixative until acquisition. Data were acquired on a BD Fortessa flow cytometer and analyzed using FlowJo software (TreeStar).

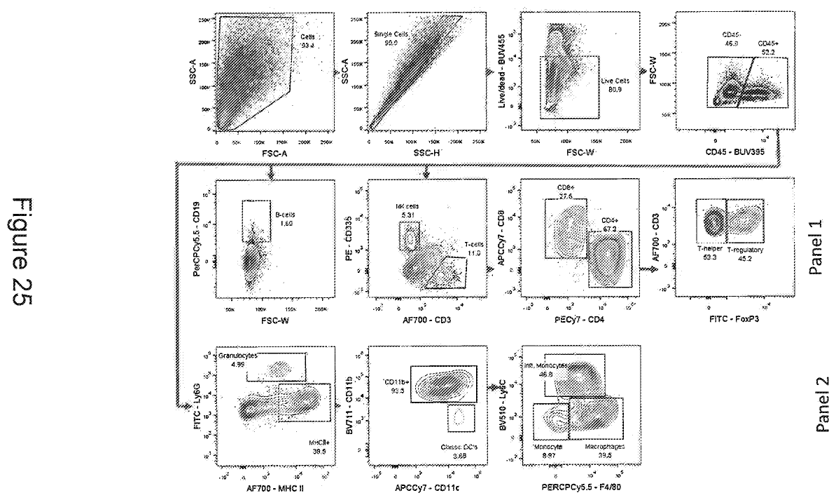

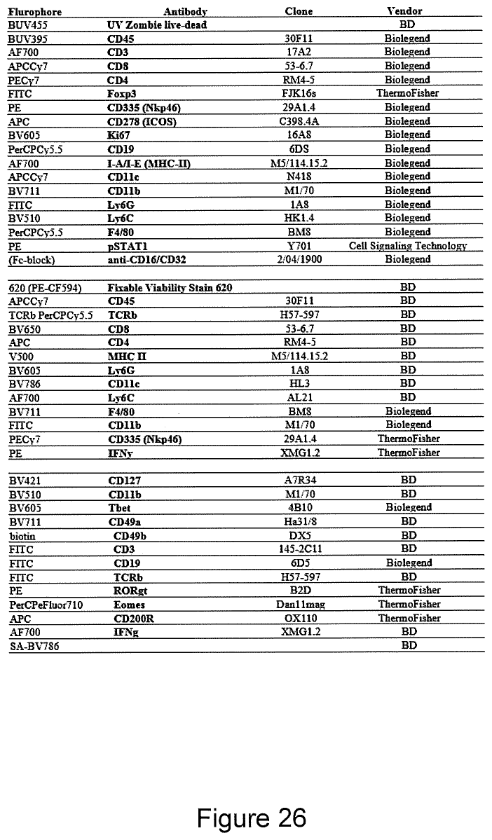

Cell populations were defined as: Monocytes (CD11b⁺, MHCII^{+/-}, Ly6C⁺, F4/80⁻, CD11 c⁻); Macrophages (CD11b⁺, MHCII⁺, F4/80⁺, CD11 c^{+/-}, Ly6C⁻, Ly6G⁻); Immature myeloid cells (CD11b⁺, MHCII⁻, F4/80⁺, Ly6C⁻, Ly6G⁻); Neutrophils/granulocytes (CD11b⁺, Ly6G⁺, MHC⁻, Ly6C^{int}, F4/80⁻); CD8 T cells (CD3⁺, CD8⁺); CD4 helper T cells (CD3⁺, CD4⁺ FoxP3⁻); Treg (CD3⁺, CD4⁺, FoxP3⁺); NK cells (CD335⁺, CD3⁻); and B Cells (CD19⁺, CD3⁻). See FIG. 25 for gating strategies employed. See also FIG. 26 for a list of antibodies use in cell detection in the flow cytometry method employed.

For detection of INFγ, cells were incubated with brefeldin A at 37° C. for 4 hours. Fixable Viability Stain 620 (BD) was used to identify dead cells. Cells were surface stained with antibodies for 20 minutes at RT. Following permeabilization with Cytofix/Cytoperm (BD), cells were stained with INFγ (ThermoFisher) in permwash (PBS/0.1% saponin (Sigma-Aldrich)) for 30 minutes on ice.

For detection of phospho-STAT1, dissociated tumours were stained with surface antibodies, fixed with 1.5% formaldehyde for 10 minutes, then permeabilized with ice-cold methanol at 4° C. for 10 minutes. Cells were stained with a rabbit-anti-pSTAT1 antibody (Tyr701) -PE (Cell Signaling Technology) for 30 minutes at room temperature (RT).

For detection of intracellular NK/ILC markers, cells were permeabilized using FoxP3 buffer Kit (ThermoFisher).

### 9. ICB Sensitizing Drug Dosing Schedules

Dosing with pretreatment drugs commenced on day 15 for AB1 and Renca, on day 10 for AE17 or on day 8 for B16.

IFNγ (Shenandoah Biotechnology) was dosed s.c. into tumour area at 50,000 units daily for 3 days.

Poly-I:C (HMW, Invivogen) was dosed s.c. into tumour area at 50 μg daily for 3 days.

The anti-IL10 hybridoma (clone JES5.2A5) was cultured in IMDM containing 1% of FCS and gentamycin. Clarified supernatants were used to purify the antibody using affinity chromatography. The antibody was sterile formulated in PBS. Anti-IL10 antibody was dosed i.p. at 0.5 mg/mouse daily for 3 days.

Regime for dosing mice with retinoids is described in more details below in part 16 of this example.

ICB agents were dosed 3 days after pretreatment drug schedule for example to give ample time to exert their biologic effect; day 20 for AB1 and Renca, day 15 for AE17 or day 13 for B16. For the ICB only group, dosing began at the same time as the pretreatment drug dosing commenced in the other arms.

### 10. NK Cell Depletion

To investigate the impact of NK cell depletion, the timing of administration of ICB antibodies was earlier whereby ICB antibodies were administered early i.e., day 5 (AB1) or day 7 (Renca) to demonstrate the greatest impact on response rate and to give a high background response rate. Anti-Asialo-GM1 (Wako Chemicals) was dosed at 20 μg in 50 μl of saline, and injected i.v. 3 days prior to ICB administration (i.e., on day 2 (AB1) or on 4 (Renca)), to have time to exert a biological effect on the tumour microenvironment. NK cell depletion was verified by flow cytometric analysis of peripheral blood using antibodies for CD45 and CD335. Antibodies for CD3, CD4, CD8, ICOS and Ki67 were also used by the inventors found no depletion of CD8 or CD4 T cells, nor decrease in number of activated cells.

### 11. RNA-Seq Data Analysis

Read libraries were quality assessed using FastQC (Andrews, S. (2010) FastQC A Quality Control tool for High Throughput Sequence Data, available online at: https://www.bioinformatics.babraham.ac.uk/projects/fastgc/) (v0.11.3) and mapped to the mouse genome (mm11) at both the transcript and gene level using HISAT2 (v2.0.4) (Kim, D., et al., (2015) Nat Meth 12, 357-360). Gene-level quantitation (counts) of aligned reads was performed using SummerizeOverlaps (Lawrence, M. et al. (2013) PLOS Computational Biology 9, e1003118), and transcript discovery and quantification using Stringtie (v1.3.0) (Pertea, M. et al. (2015) Nature biotechnology 33, 290-295,) and Ballgown (Frazee, A. C. et al. (2015) Nature biotechnology 33, 243-246).

Principal component analysis (PCA) was performed to visualise the major contributions of variation within the data using the prcomp ( ) function within R (v3.3.3). Gene count data was transformed for PCA employing the variance stabilising transformation (Anders, S. & Huber, W. (2010) Genome Biology 11, R106). The top 1000 most variable genes across samples (those with the greatest median absolute deviation) were selected for PCA. Differentially expressed genes were identified between immunotherapy non-responders and responders using DESeq2 (Love, M. I., et al., (2014) Genome Biology 15, 550). P-values were adjusted for multiple comparisons using the Benjamini-Hochberg method (Benjamini, Y. & Hochberg, Y. (1995) Journal of the Royal Statistical Society. Series B (Methodological) 57, 289-300), and those <0.05 were considered significant (Y. Benjamini, & Y. Hochberg (1995), Controlling the False Discovery Rate: A Practical and Powerful Approach to Multiple Testing. Journal of the Royal Statistical Society. Series B (Methodological) 57, 289-300).

Differentially expressed genes were uploaded to InnateDB (www.innatedb.com) along with associated gene expression data. A list of pathways mapping to the uploaded genes was returned, and pathway analysis was undertaken to determine which pathways were significantly overrepresented in the up- and downregulated gene data sets. InnateDB simultaneously tests for overrepresentation of DE genes in more than 3,000 pathways, from which the KEGG (www.genome.ad.jp/kegg/), and Reactome (www.reactome.org/) databases were looked at. The Benjamini and Hochberg (BH) FDR correction was applied to correct for multiple testing.

The weighted gene co-expression network analysis (WGCNA) algorithm (Langfelder, P. & Horvath, S. (2008) BMC Bioinformatics 9, 559-559) was used to construct a signed network across all samples and identify clusters (modules) of genes with highly correlated patterns of gene expression. To prepare the data for WGCNA inventors: (i) applied a variance stabilising transformation to normalise the data, (ii) filtered out genes expressed at a low level (only those with at least 10 counts per sample were retained), (iii) removed genes without an official MGI symbol and (iv) removed genes with low variability by applying the variance Based filter ( ) function within the DCGL package (significance threshold set to 0.01) (Yang, J. et al. (2013) PLOS ONE 8, e79729). The resulting set of 6026 genes were used as input for network construction. Network modules of co-expressed genes identified by WGCNA were tested for enrichment of differentially expressed genes between immunotherapy non-responders and responders by plotting the −log₁₀ p-values derived from the DESeq2 analysis, on a module-by-module basis. Protein-protein interaction networks of the differentially expressed genes were created using Innate DB network analysis tool (unfiltered), and network created using Network Analyst (J. Xia et al., (2014) Nucleic Acids Res 42, W167-174), with STRING interactome, confidence cut-off 900 (D. Szklarczyk et al., (2015) Nucleic Acids Res 43, D447-452). Rendering of module interactions was performed with Cytoscape software. Network modules of interest and differentially expressed genes (filtered to include those with an absolute fold-change in expression >2) were also analysed within Ingenuity Systems (Krämer, A., et al., (2014) Bioinformatics 30, 523-530) to identify associated upstream transcriptional regulators, using right-tailed Fisher's exact tests and default settings for other options. P-values <0.05 were considered significant. An activation z-score was also calculated for each upstream transcriptional regulator by comparing their known effect on downstream targets with observed changes in gene expression. Those with activation z-scores ≥2 or ≤2 were considered “activated” or “inhibited”, respectively.

The CIBERSORT algorithm (Newman, A. M. et al. (2015) Nature Methods 12, 453) was used to estimate the relative proportions of 25 mouse hematopoietic immune cell types based on the transcriptomic profiles of each sample, where the 511 mouse-gene signature developed by Chen et al was used as a reference (Chen, Z. et al. (2017) Scientific reports 7, 40508). Inventors broadly classified the 25 cell types into 12 major populations by collapsing several related sub-populations as follows: B cells comprise memory, naïve and plasma cells; CD8 T cells comprise memory, naïve and activated cells; CD4 T cells comprise memory, naïve, follicular, Th1, Th2 and Th17 cells; Macrophages comprise M0, M1 and M2 phenotypes; NK cells comprise activated and resting cells; DCs comprise activated and immature cells. Prior to analysis, transcript level data was library size and gene length normalised using the ballgown package (Frazee, A. C. et al. (2014) bioRxiv) resulting in Fragments Per Kilobase of transcript per Million mapped reads (FKPM). Individual transcripts were collapsed to gene level data based on the mean FPKM value using the aggregate ( ) function. Finally, the data was filtered to retain genes with an FPKM value >0.3 in at least 8 samples (being the smallest experimental group size).

The Broad Institute gene set enrichment analysis (GSEA) software (A. Subramanianet al., (2007) Bioinformatics 23, 3251-3253) was used to run analyses on normalized gene expression data with prefiltering for low counts. The Hallmarks gene set database which uses 50 MSigDB Hallmarks gene sets (A. Liberzon et al., (2015) Cell Syst 1, 417-425) and a STAT1 signature were used. The STAT1 signature was derived from Care et al (M. A. Care, D. R. Westhead, R. M. Tooze, (2015) Genome Med 7, 96), defined based on variance-ranked Spearman correlations of gene expression across 11 DLBCL datasets, selecting strongly correlated genes (cut-off median correlation score >0.6). A total of 1000 permutations were performed. All other default parameters were used. Gene sets enriched at a nominal P value <0.05 and FDR<0.25 were considered significant.

12. Survival Analysis for Patients with STAT1 Signature Enrichment

To define the subset of patients with STAT1 pathway activation for survival analysis, inventors closely followed the Classification Algorithm Based on a Biological Signature (F. Reynier et al., (2011) PLoS One 6, e24828.). The previously defined STAT1 gene set from Care et al (M. A. Care, D. R. Westhead, R. M. Tooze, (2015) Genome Med 7, 96) was used and leveraged RNAseq data from the Imvigor210 trial (S. Mariathasan et al., (2018) Nature 554, 544-548), available as an R data package (Imvigor210CoreBiologies). The dataset contains gene count data of 348 urothelial cancer patients treated with the anti-PD-L1 antibody atezolizumab. Patients were classified by best response: 25 complete responders (CR), 43 partial responders (PR), 63 with stable disease (SD), and 167 progressors (PD). 50 patients did not have evaluable disease (NE). From this RNAseq data, two prototype vectors were defined based on mean expression values of genes in this STAT1 gene set; a “responder” prototype from radiological complete responders and partial responders and a “progressor” prototype based on progressor samples. For each patient, expression profiles of the STAT1 gene set were compared to the two prototype vectors. Similarity was calculated based on Pearson correlation coefficient and a decision score formulated based on the ratio of correlations. This decision score was used to classify all patients in the Imvigor210 dataset regardless of response, with a decision score >1 denoting higher activation of the STAT1 pathway. Survival analysis was performed using the Logrank test, dividing patients based on this decision score, with a decision score >1 denoting higher activation of the STAT1 pathway; a Kaplan-Meyer survival curve was constructed.

13. Immunohistochemistry for STAT1 and pSTAT1

For immunohistochemical staining of STAT1 and pSTAT1, slides of 4-μm thickness were cut from formalin-fixed, parafin-embedded (FFPE) tissue blocks. Subsequently, slides were deparafinized in two changes of xylene for about 5 min followed by rehydration in changes of 100%, 96%, 70% and 40% ethanol and distilled water. Next, for STAT1 staining, antigen retrieval using 10 mM citrate buffer (pH 6.0) was performed for about 10 min at 121° C. using a pressure cooker (the PT-module, Thermo scientific, labvision). For pSTAT1 staining, slides were sub-boiled in 1 M EDTA buffer (pH 8.0) for about 10 minutes in a microwave. Slides were rinsed in TBST and the endogenous peroxidase was blocked using 3% hydrogen peroxidase (Sigma) in distilled water. Sections were washed again in TBST and blocked with goat serum (Vectastain) diluted in PBS as per manufacturer's instructions. Primary antibody, STAT1 or pSTAT1 (Cell Signaling, dilution: 1/800 and 1/200 respectively), was incubated for 60 minutes at room temperature (RT). Sections were washed and secondary antibody, goat anti-Rb IgG-HRP (Santa Cruz), was incubated for about 30 minutes at RT, before washing again. Betazoid DAB chromogen (Biocare Medical) was prepared as per manufacturer's instructions. Chromogen was applied, sections were monitored for the development of a brown colour and the reaction was stopped with H₂O. Sections were counterstained with haematoxylin and rehydrated before mounting coverslips with Pertex. An experienced pathologist scored the sections by assigning an estimated proportion of positive tumour cells (positivity defined as moderate or strong nuclear staining for pSTAT1 and moderate or strong nuclear and cytoplasmic positivity for STAT1), while blinded to treatment outcome.

### 15. Induction of IFN Alpha/Beta Signalling for Sensitising Tumour Cellular Microenvironment to ICB.

To test whether the mechanism underlying the priming effect of Poly-I:C was mediated through IFN alpha/beta induction (as discussed in Example 6), priming experiments were repeated while adding blocking antibodies against the IFN alpha/beta receptor (IFNAR). C57BL/6 AE17 mesothelioma tumour-bearing mice were treated with CPB on day 22 after tumour inoculation, with or without pretreatment of 3 days of 50 μg s.c. Poly-I:C in the tumour area (day 17-19), with or without blocking antibodies against IFNAR (e.g., rabbit monoclonal antibody MAR1-5A3 as sourced from BiXcell) (0.5 mg i.p., 3 times/week) starting at the same time.

To verify the potential of recombinant IFN alpha to induce the response-associated phenotype (i.e., to sensitise the tumour cellular microenvironment to ICB) similarly to Poly-I:C, AE17 mesothelioma tumour-bearing mice were pretreated with Poly-I:C or recombinant IFN alpha (: R&D systems) intratumourally for 3 days (or PBS control). Tumours were then dissociated and stained for NK marker CD335, pan-leukocyte marker CD45 and pSTAT1 and subject to analysis by flow cytometry.

### 16. Sensistization of Tumour Microenvironment to Retinoids.

As tretinoin (all-trans retinoic acid) was identified as one of the top predicted upstream regulators of the response-associated gene expression signature with a p value of 0.000954 and a z-score of 2.565, investors sought to test the ability of tretinoin to sensitize the tumour microenvironment to checkpoint blockade (ICB). To this end, mice bearing AB1-HA mesothelioma tumours were with ICB (anti-CTLA4 and anti-PD-L1 antibodies) as described earlier with or without tretinoin (Sigma-Aldrich). The administration of tretinoin to the mice started either 3 days before ICB or at the same time as the ICB (arm 6). Tretinoin was dosed at three dosages; 10 mg/kg (arm 3), 5 mg/kg (arm 4) or 1 mg/kg (arm 5) and was given for 9 days in total.

To investigate whether tretinoin would be able to achieve sensitization of tumours to ICB when used in combination with only a single ICB antibody, AB1-HA tumour-bearing mice were treated with tretinoin 10 mg/kg i.p. starting on day 6 for a total of 9 days, and an anti-PD-L1 antibody (200 μg, i.p.) was given on days 8, 10 and 12.

For the purpose of (i) testings whether other retinoid compounds, i.e., beside tretinoin, would also improve the efficacy of checkpoint blockade by sensitisation of tumours to ICB, and (ii) to demonstrate utility of retinoid in a variety of other tumour models, and (iii) to test whether the use of retinoids in sensitizing tumours to ICB could be achieved by oral dosing, Renca kidney cancer-bearing mice were exposed to ICB treatment with anti-CTLA4 and anti-PD-L1 antibodies (anti-CTLA4 dosed day 12, 100 μg i.p.; anti-PD-L1 dosed days 12, 14, 16, 100 μg i.p.) in combination with retinoids which were dosed daily for 6 days through oral gavage starting on day 9. The following retinoids and dosages were employed: Bexarotene (Saphire Bioscience) was dosed at 100 mg/kg; tretinoin and isotretinoin (Sigma-Aldrich) were all dosed at 10 mg/kg.

To test whether retinoids induce the ICB response-associated phenotype characterised by increased NK cells, inflammatory markers and pSTAT1 positive leukocytes in the tumour, Renca kidney cancer-bearing mice were treated for 5 days with oral tretinoin (10 mg/kg) starting on day 12 and harvested tumours on day 15 for flow cytometry. In subsequent experiments AB1-HA mesothelioma or CT26 colorectal cancer tumour-bearing mice were treated with tretinoin for 3 days at 10 mg/kg i.p., starting on day 10 and harvested tumours on day 13 for flow cytometry and immunohistochemistry.

### 17. Statistics

The sample size calculation for in vivo mouse experiments was based on prior experiments in it was found that the median survival time on the control treatment (ICB alone) was 35 days (W. J. Lesterhuis et al., (2015) Sci Rep 5, 12298). Using a proportional hazards model it was determined that, if the true hazard ratio (relative risk) of control subjects relative to experimental subjects is 5, it would be need to study 10 experimental subjects and 10 control subjects to be able to reject the null hypothesis that the experimental and control survival curves were equal with probability (power) 0.8. The type I error probability associated with this test of this null hypothesis was 0.05.The sample size for the bulk RNAseq experiments was estimated using the method developed by Hart et al (S. N. Hart et al., (2013) Comput Biol 20, 970-978); for sample sizes of n=12 and a within group coefficient of variation of 0.3 there was >90% power to detect a 1.7-fold change in gene expression. Differences in population frequencies in responders and non-responders using flow cytometry and CIBERSORT were determined using Mann-Whitney U testing on means.

Prism software (GraphPad) was used to analyse tumour growth and to determine statistical significance of differences between groups by applying a Mann-Whitney U test. P-values were adjusted for multiple comparisons using the Benjamini-Hochberg (B-H) method; those <0.05 were considered significant. The Kaplan-Meir method was used for survival analysis, and p-values were calculated using the log-rank test (Mantel-Cox).

### Example 3: Tumours Derived from Clonal Cancer Cell Lines, Grown in Inbred Mouse Strains Display Two Distinct Gene Signatures, Predicting Sensitivity to ICB

This example demonstrates that it is possible to differentiate microenvironments of neoplastic cell populations and tumours that were going to be respond to immunotherapy with ICB agents from non-responders even before they are treated with the immunotherapy. Equally, this example also demonstrates that it is possible to differentiate those subjects which are predicted to be responders to immunotherapy with ICB agents from non-responders. Accordingly, this example also demonstrates that it is possible to predict whether or not a neoplastic cell population or tumour or a subject having the neoplastic cell population or tumour is going to response to immunotherapy with ICB agents even before treatment with the immunotherapy.

Previous attempts to define a signature predicting response to ICB have not been successful. There are many potential reasons why a definitive biomarker for the response to ICB has not emerged, including differences in host genetics, environmental factors and the diverse genetic and cellular make up of cancers between patients. Interestingly, even inbred mouse strains bearing transplantable tumours display a dichotomous outcome after immunotherapy (see FIG. 1A). This is surprising since the genomes of these mice are nominally identical and the tumours are derived from a clonal cell line, thus excluding the possibility that a difference in tumour rejection antigen expression caused these disparate responses. In these experiments, the mice were of the same age and gender, and were kept under controlled, pathogen-free conditions, and received identical treatment. Yet, they responded very differently. Without being bound by any theory or any mode of action, it is possible that differences in outcomes between animals were related to differences in T cell repertoire, which was not encoded in the germline, (Madi, A. et al. (2017) Elife 6) or stochastic immunological events; (Germain, R. N. (2001) Science 293, 240-245). Regardless of the cause of this dichotomy, the inventors reasoned that this dichotomy in response to ICB observed inbred mouse strains bearing transplantable tumours which are derived from a clonal cell line, would allow to assess potentially small differences in microenvironmental regulation of therapeutic responses in a controlled background.

To assess the differences in tumour microenviromental regulation of therapeutic responses in a controlled background, inbreed mice strains (BALB/cArc, BALB/cJAusb and C57BL6/J mice 8-12 weeks of age) were inoculated bilaterally with either AB1 mesothelioma cells or Renca kidney cancer cells and treated them with anti-CTLA4 and anti-PD-L1 antibodies as described in Example 2. As demonstrated in FIGS. 1A, 1B and 1C, this resulted in symmetric, yet dichotomous responses i.e., tumours either responded or did not respond (FIG. 1B, FIG. 1C).

These mouse models thus allowed the biological assessment of a whole tumour at any time point, by surgically removing it, while still being able to infer the therapeutic fate of that tumour if it had been left in situ by monitoring the fate of the remaining contralateral tumour. Using these models, the pre-treatment tumour cellular microenvironment was characterised. One tumour was surgically for detailed analysis using bulk RNAseq, flow cytometry and single cell RNAseq shortly before administering ICB agents such as anti-CTLA4 and anti-PD-L1 antibodies. The remaining tumour was assessed to determine the therapeutic outcome of ICB agents such as anti-CTLA4 and anti-PD-L1 antibodies (FIG. 1D).

As shown in FIG. 1E and FIG. 1F use of principle component analysis (PCA) of bulk RNAseq data (as outlined in Example 2) revealed that responsive and non-responsive neoplastic cell population such as tumours clustered separately in both models. Unsupervised hierarchical clustering of the top differentially expressed genes resulted in clear separation of responsive and non-responsive tumours (FIG. 1G, FIG. 1H).

Accordingly, the results shown in FIG. 1 confirmed the upregulation of several response-associated genes at the protein level in the cellular microenvironment of tumours which respond to ICB agents including PD-L1 protein, which is in accordance with clinical studies in many different cancers (FIG. 1i) (Herbst, R. S. et al. (2014) Nature 515, 563-567; and Reck, M. et al. (2016) N Engl J Med 375, 1823-1833). More specifically, the results provided herein demonstrate that untreated, ostensibly identical tumours in inbred mice displayed a surprising heterogeneity in gene expression profiles, allowing to differentiate neoplastic cell populations and/or tumours as well as animals having such neoplastic cell populations and/or tumours that were going to be responders from non-responders even before they were treated with the immunotherapy.

### Example 4: ICB Responsive Tumours are Characterized by an Inflammatory Microenvironment Driven by STAT1

This example demonstrates that STAT1 activation is a driver of the ICB response-associated tumour microenvironment and can serve as a potential biomarker to identify neoplastic cell population and/or tumours and/or patients having such neoplastic cell populations and/or tumours more likely to respond to ICB immunotherapy.

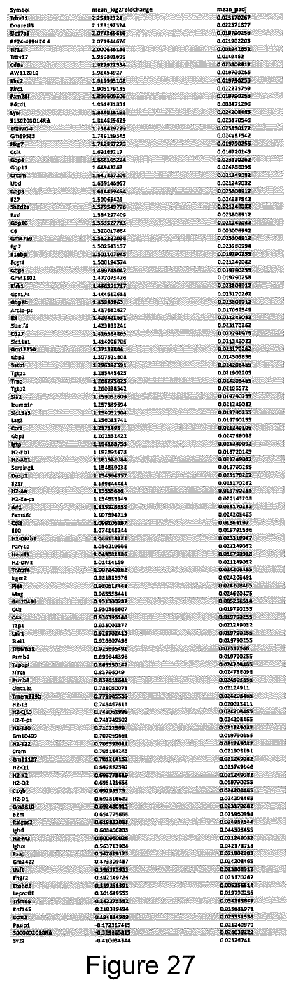

The inventors aimed to gain insight into the biological relevance of the differentially expressed genes in responsive and non-responsive tumours. Firstly, they noted a striking difference between the two models in terms of differentially expressed genes between responders and non-responders, with AB1 tumours having more than 10,000 genes differentially expressed, while in Renca tumours only 127 genes were differentially expressed. However, the inventors also noted that the majority of the 127 genes which were differentially expressed in Renca tumours overlapped with AB1 (118 genes), resulting in a refined response-associated signature. A list of these 118 genes which were found to be differentially expressed in both Renca tumours and AB1 tumours are shown in FIG. 27.

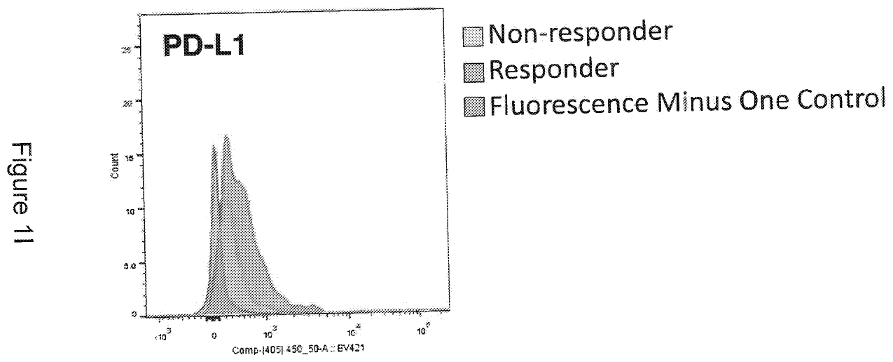

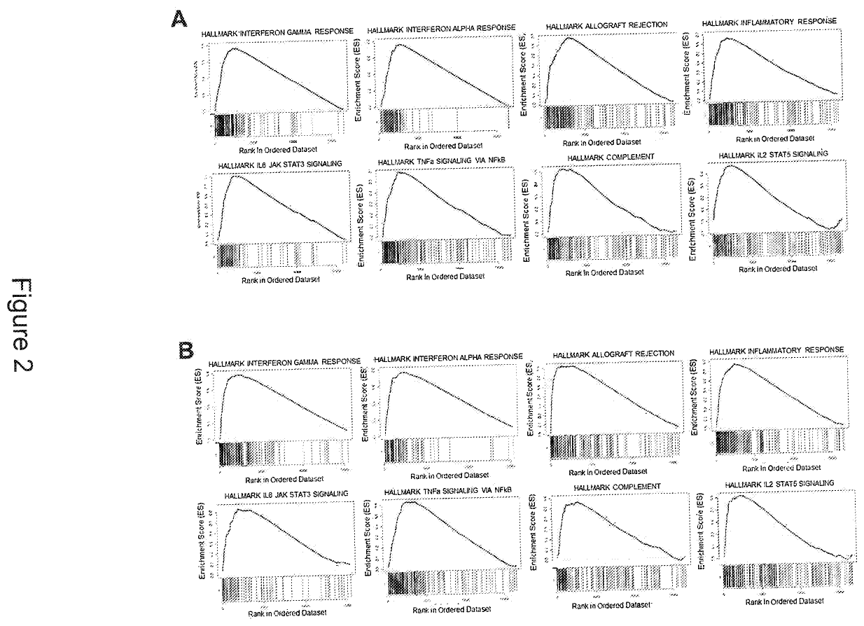

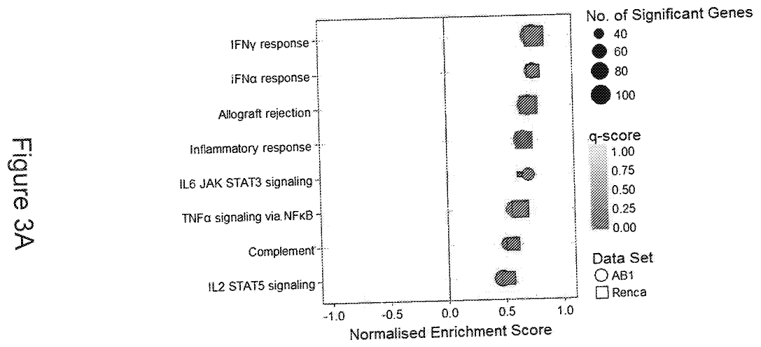

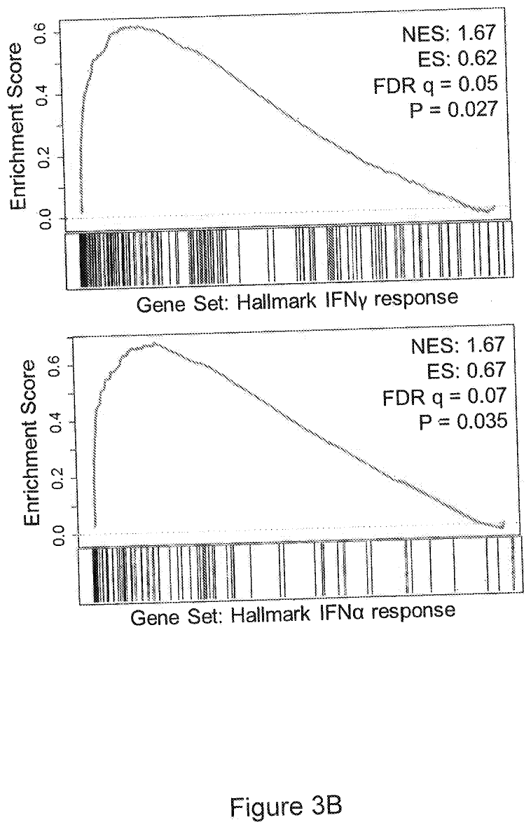

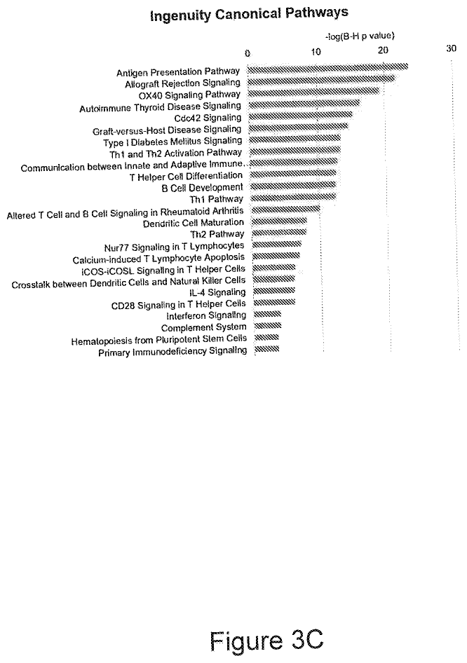

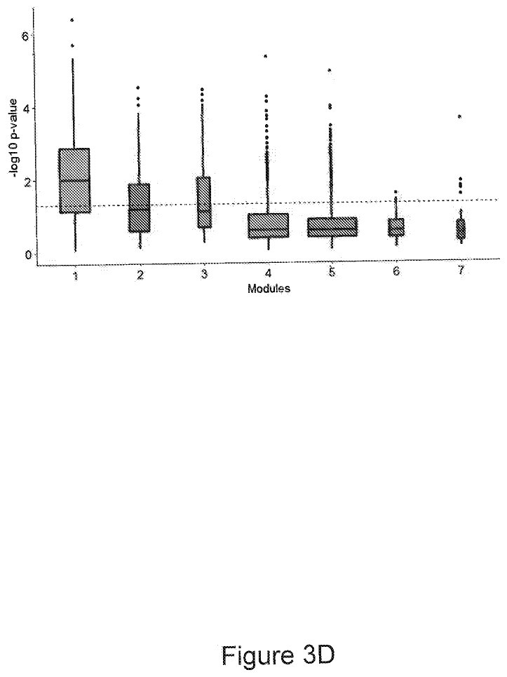

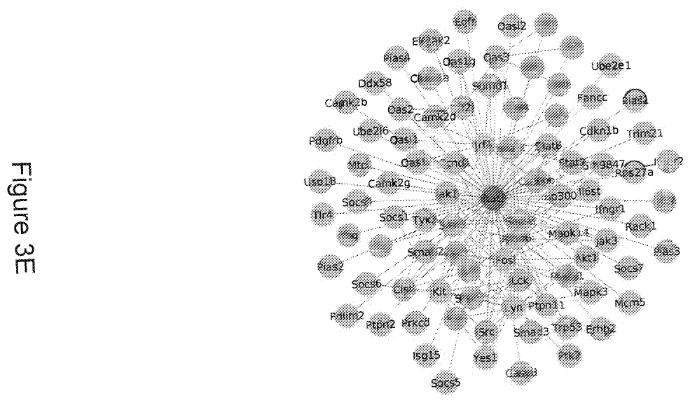

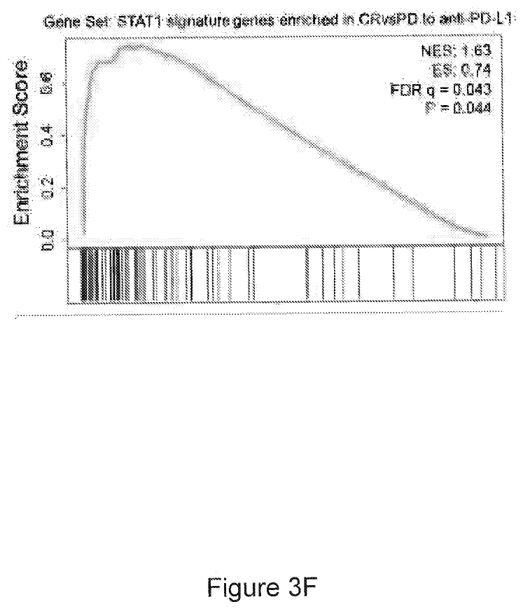

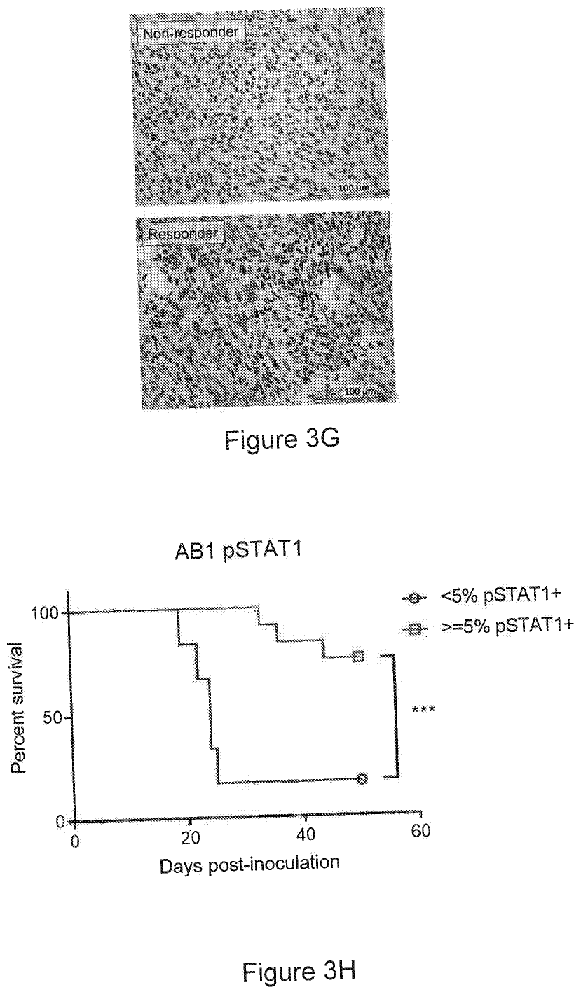

To further characterize the overarching biological processes associated with this refined response-associated signature with differentially expressed genes between responders and non-responders, the inventors analysed the distribution of a well-characterized and curated collection of 80 ‘hallmark’ gene sets using gene set enrichment analysis (GSEA) (Liberzon, A. et al. (2015) Cell Syst 1, 417-425) (see FIG. 3A and FIG. 2). As shown in FIG. 3A and FIG. 2 This revealed enrichment of genes associated with inflammatory response, allograft rejection, type I and II interferon response, and IL6-JAK-STAT3 signalling in responsive tumours in both murine models. The inventors then performed GSEA of the 80 Hallmark gene sets on a publically available dataset generated from pretreatment tumour biopsies from a cohort of urothelial cancer patients treated with ICB agents, the PD-L1 antibody atezolizumab who went on to respond (S. Mariathasan et al., (2018) Nature 554, 544-548.). These gene sets were indeed also enriched in the pretreatment tumour biopsies from who went on to respond, as shown in FIG. 3B, thereby validating the translational relevance of the findings of this example.

Pathway analysis of common differentially expressed genes in the two models identified antigen presentation and Th1 type immune responses as the most enriched pathways in responsive tumours (see FIG. 3C). These data accord with published data in human melanoma patients (M. Ayers et al., (2017) J Clin Invest 127, 2930-2940), and suggest that the activation of inflammatory pathways, in particular the IFN response, renders tumours sensitive to ICB in animal cancer models and patients alike.

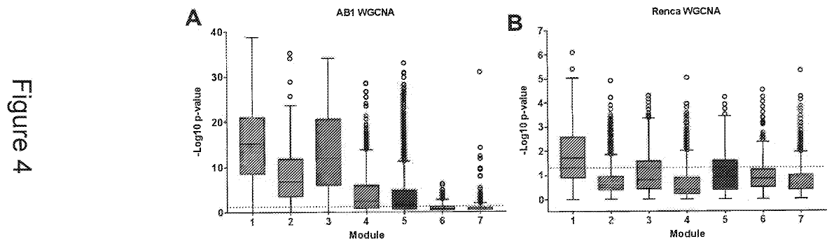

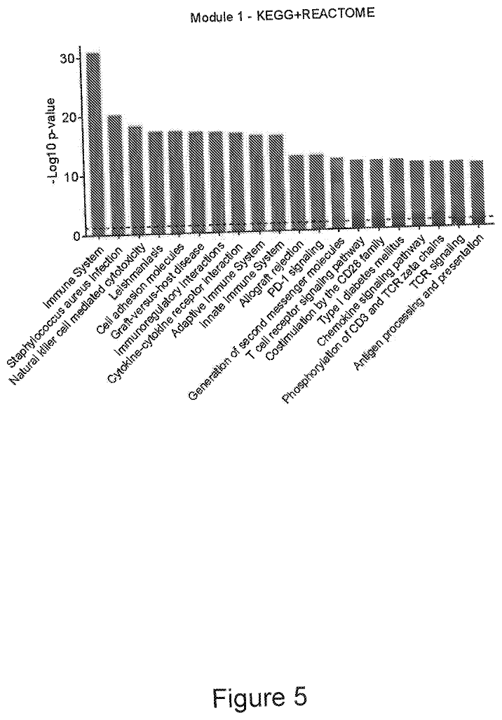

Next, weighted gene correlation network analysis (WGCNA) was used essentially as described in P. Langfelder and S. Horvath (2008) BMC Bioinformatics 9, 559 to map the molecular networks underlying responsiveness to ICB. This identified 7 modules of highly co-expressed genes operating within tumours, of which one was significantly upregulated in responsive tumours in both models; see module 1 in FIG. 3D and FIG. 4. This response-associated module was enriched for genes involved in adaptive immunity, in particular NK cell mediated cytotoxicity, and IFNγ, PD-1 signalling and costimulation (see FIG. 5). Based on the overall strength of correlation patterns between genes within the same module using WCGNA Network Analyst substantially as described in (J. Xia et al., (2014) Nucleic Acids Res 42, W167-174.) inventors subsequently assembled a putative network that identified STAT1 as a hub (see FIG. 3E). This result demonstrated that STAT1 was the highest connected gene in the response-associated inflammatory module. Promotors of the genes in this module were predicted t0 be significantly enriched for STAT1 binding sites, using TRANSFAC and JASPAR (p 5.7e-7; Z-score -2.12).

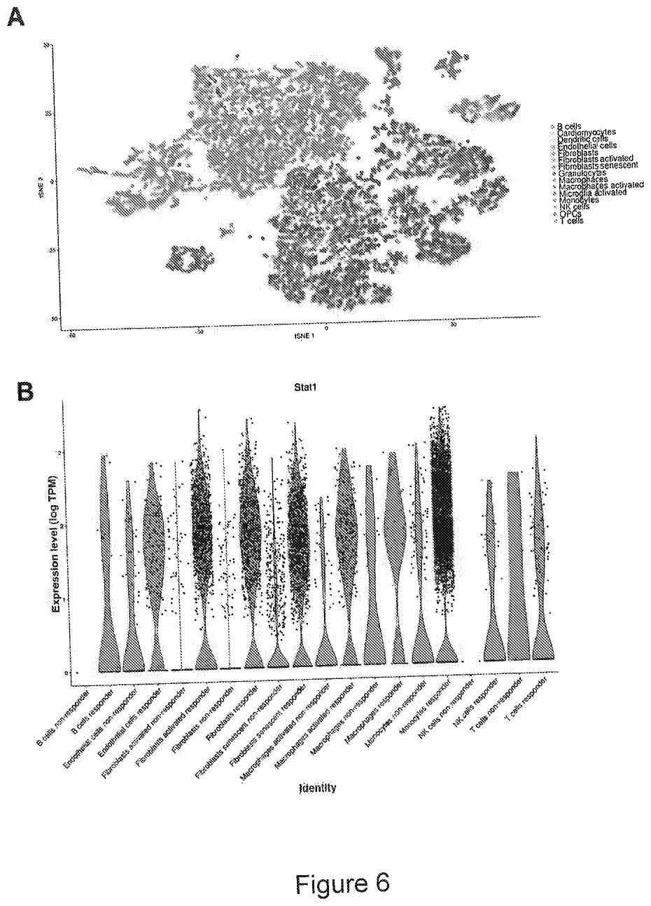

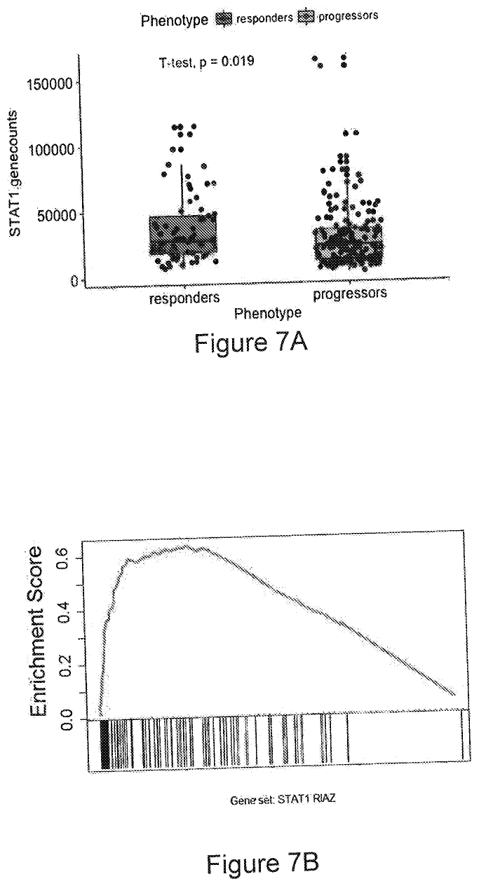

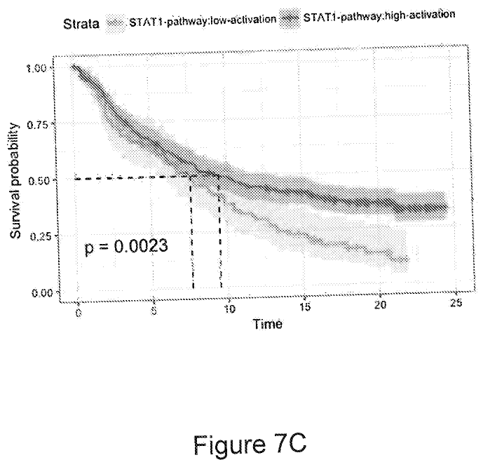

These findings were corroborated in single cell analysis of AB1 tumours, which identified greater STAT1 gene expression in both responsive and non-responsive AB1 cells (see FIG. 6). In addition, there was a significant (p=0.019) enrichment of a STAT1 signature in responsive tumours from the urothelial cancer patients treated with Atezolizumab when compared to non-responsive patients (S. Mariathasan et al., (2018) Nature 554, 544-548) as well as in a second cohort of patients with melanoma treated with nivolumab (p=0.044) (N. Riaz et al., (2017) Cell 171, 934-949 e915); see FIG. 3F, FIG. 7A and FIG. 7B.

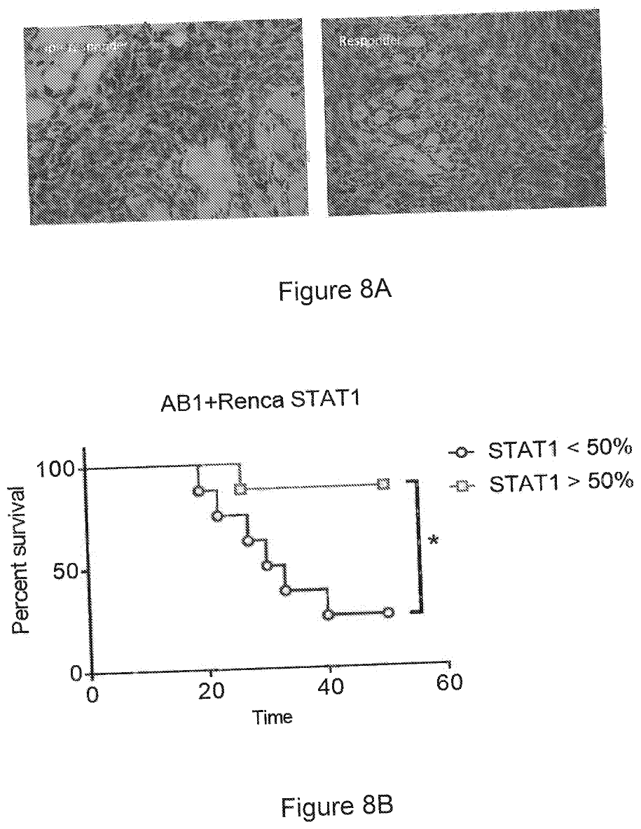

Since STAT1 activation results in nuclear translocation, inventors immunohistochemically assessed nuclear STAT1 in responding and non-responding tumours. Inventors found that the presence of nuclear (i.e., as a proxy for activation) STAT1 correlated with response to checkpoint blockade; all responding mice displayed positivity of above 50% nuclear STAT1 while none of the non-responding mice did (FIG. 8).

Since STAT1 is activated through phosphorylation, which can be assessed by immunohistochemistry, the total and phosphorylated STAT1 abundance was assessed in the mouse models described herein. As demonstrated in FIG. 3G, FIG. 3H, when the inventors used antibodies specific against phosphorylated (i.e. activated) STAT1, they found that responsive tumours had higher percentages of phosphorylated STAT1 positive cells than non-responsive tumours. Similarly, the absence of a STAT1 gene signature in human pretreatment samples was associated with a decreased progression-free survival in the urothelial cancer/atezolizumab clinical dataset (see FIG. 7C), which accords with a previous report on the predictive value of tumour STAT1 activation in melanoma patients treated with anti-PD1 (P. C. Tumeh et al., (2014) Nature 515, 568-571).

Together, these data suggest that STAT1 activation is a driver of the ICB response-associated tumour microenvironment and can serve as a potential biomarker to identify patients more likely to respond.

### Example 5: Cellular Analyses of Resistant and Sensitive Tumours Identify the Presence of NK Cells as a Prerequisite for Response to ICB

This example demonstrates that very different cellular tumour microenvironments between models are still conducive to rendering tumours responsive to ICB therapy.

This example further demonstrates that pretreatment tumour NK cell infiltration may be required for tumour response to ICB therapy.

As the inventors we observed an increase (i.e., enrichment) of genes associated with an inflammatory IFN/STAT1-driven environment in tumours responsive to ICB agents, and because it has been previously found that ‘hot’ tumours may be characterized by increased CD8 T cell infiltration (P. C. Tumeh et al., (2014) Nature 515, 568-571), the inventors examined the immune cell infiltrates of responsive and non-responsive tumours.

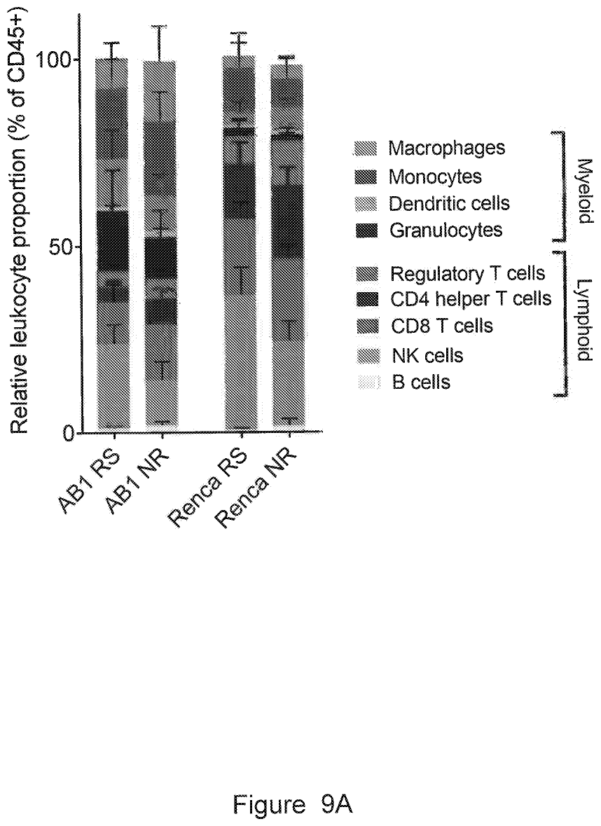

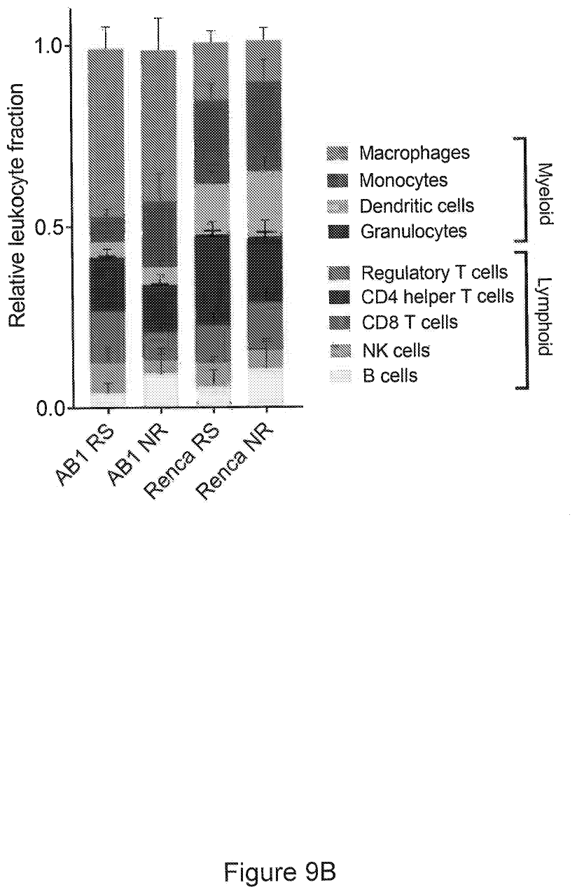

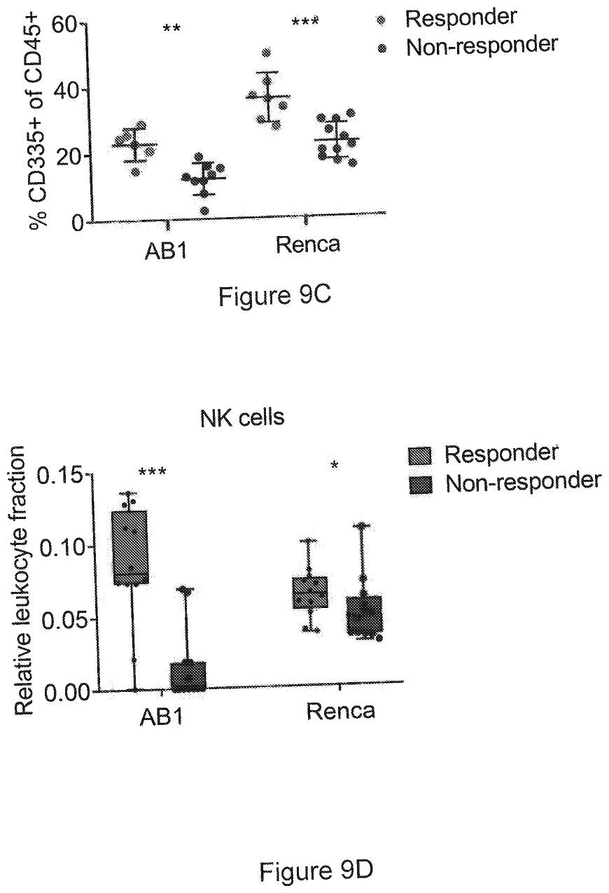

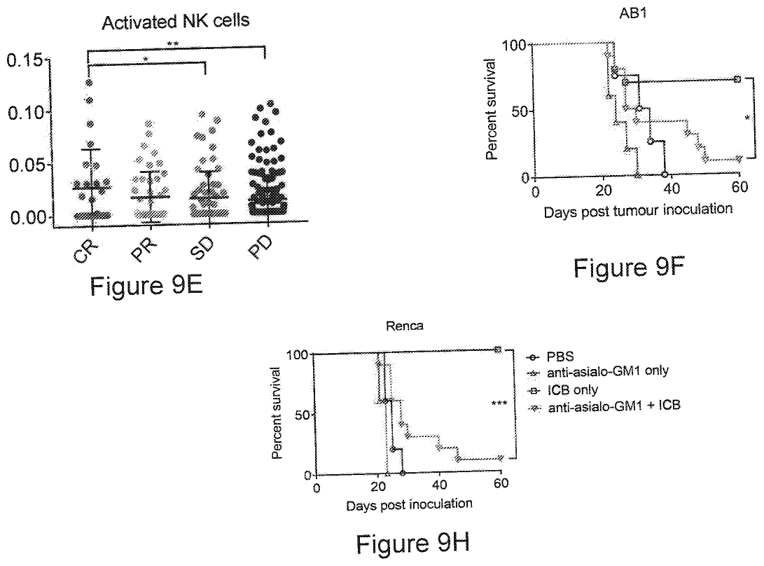

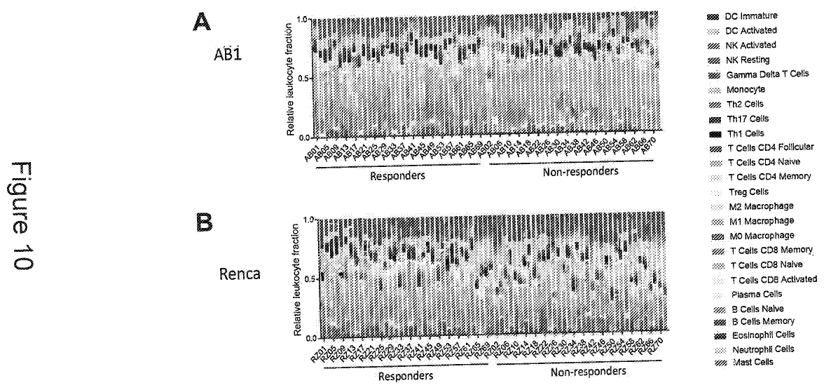

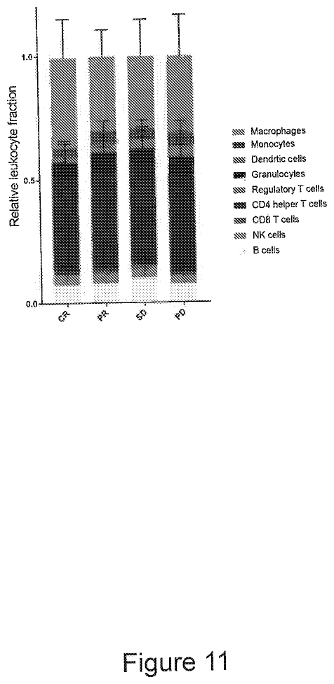

Specifically, flow cytometry was employed on dissociated tumours and the data was compared using a CIBERSORT analysis substantially as described in by A. M. Newman et al., (2015) Nat Methods 12, 453-457. In this respect, CIBERSORT is a deconvolution approach for characterizing cell composition of complex tissues from gene expression data and the inventors applied to the single cell RNAseq data obtained herein. As demonstrated in FIG. 9, AB1 tumours were characterized by a predominantly myeloid infiltrate, whilst the infiltrate in Renca tumours was mostly lymphoid (See FIG. 9A and FIG. 9B respectively). Interestingly, despite the striking differences in cellular infiltrates, these tumour mouse models respond similarly to checkpoint blockade. There were no observed consistent difference between responding tumours and non-responding tumours with regard to infiltrating CD8 or CD4 T cells, B cells, macrophages, monocytic cells or dendritic cells (See FIG. 9A and FIG. 9B, and FIG. 10). However, there was a greater proportion of NK cells in responding tumours in both tumour mouse models (in other words responding tumours has more NK cells); see FIG. 9C and FIG. 9D. The percentage of overall leukocytes was not markedly different between responsive and non-responsive tumours, as measured by CD45⁺ cells of all tumour-containing cells using flow cytometry (see FIG. 11). The percentage of NK cells of all tumour cells was significantly (p<0.0001) increased, as measured by CD335⁺/CD3⁻ cells of all tumour-containing cells using flow cytometry (FIG. 11).

To assess whether tumour NK cell enrichment could be relevant in humans, the inventors interrogated a gene expression dataset of patients with melanoma, head and neck cancer or lung cancer treated with the PD-1 blocking antibodies Nivolumab or Pembrolizumab (Prat, A. et al., (2017) Cancer Res 77, 3540-3550). Using gene set enrichment analysis (GSEA), the inventors found that an NK-specific gene set (Bezman, N. A. et al., (2012) Nat Immunol 13, 1000-1009) was markedly associated with response in these patients (data not shown). In addition, the inventors used CIBERSORT analysis to interrogate another human patients gene expression dataset of the urothelial cancer patient cohort, obtained prior to treatment with the anti-PD-L1 antibody Atezolizumab, as an ICB therapy (S. Mariathasan et al., (2018) Nature 554, 544-548 (2018), and found that responding patients had markedly higher numbers of NK cells in their tumours (FIG. 9E, FIG. 11), similar to the mouse models (FIG. 9C). Accordingly, these results demonstrate that also in humans gene sets specific for activated NK cells are noticeably correlated with response to ICB therapy.

To test whether NK cell infiltration of the pretreatment tumour microenvironment was required for response in the tumour mouse models described herein, NK cells were depleted (with a single injection of anti-asialo GM1) three days before ICB treatment in both AB1 tumours and Renca tumours which resulted in a markedly diminished response (FIG. 9F and FIG. 9G). These data demonstrate that very different cellular tumours microenvironments between models are still conducive to a response to ICB. Furthermore, the data also demonstrates that pretreatment tumour NK cell infiltration is required for response to ICB.

### Example 6: Development of a Treatment Regime Commencing Prior to the ICB Therapy in Order to Sensitize Neoplastic Cell Populations and/or Tumours to ICB

This example demonstrates application of clinically available therapeutics which result in marked sensitization of neoplastic cell populations and/or tumours to ICB agents which commences prior to the ICB therapy and may be continued during ICB immunotherapy.

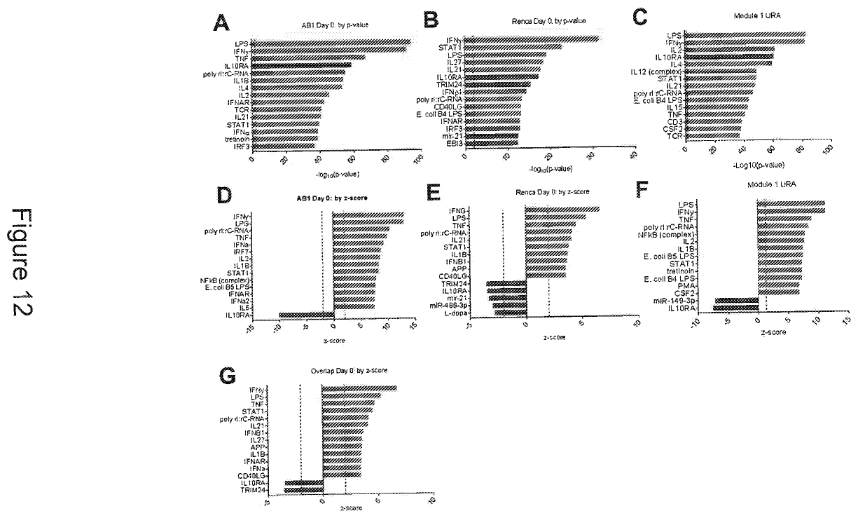

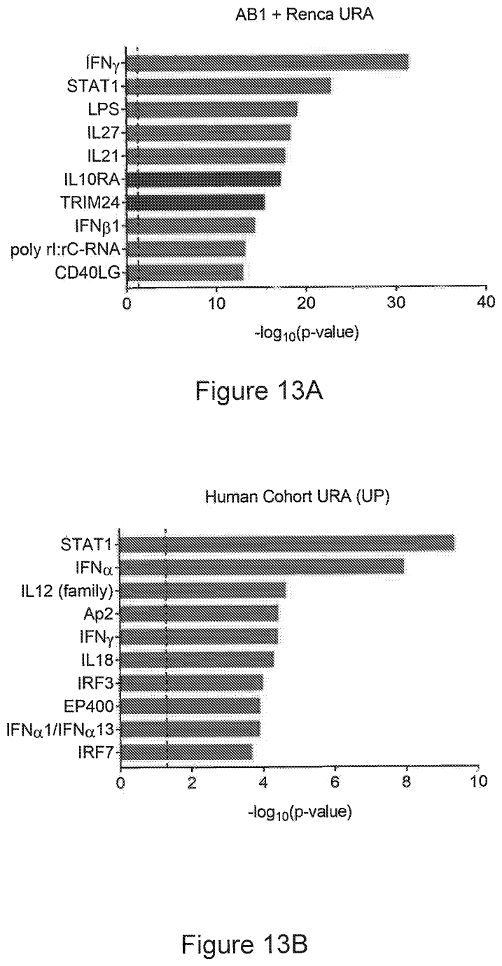

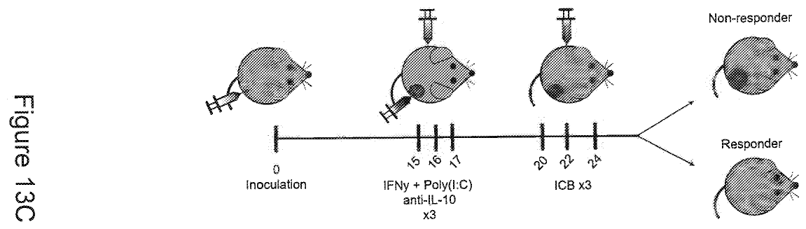

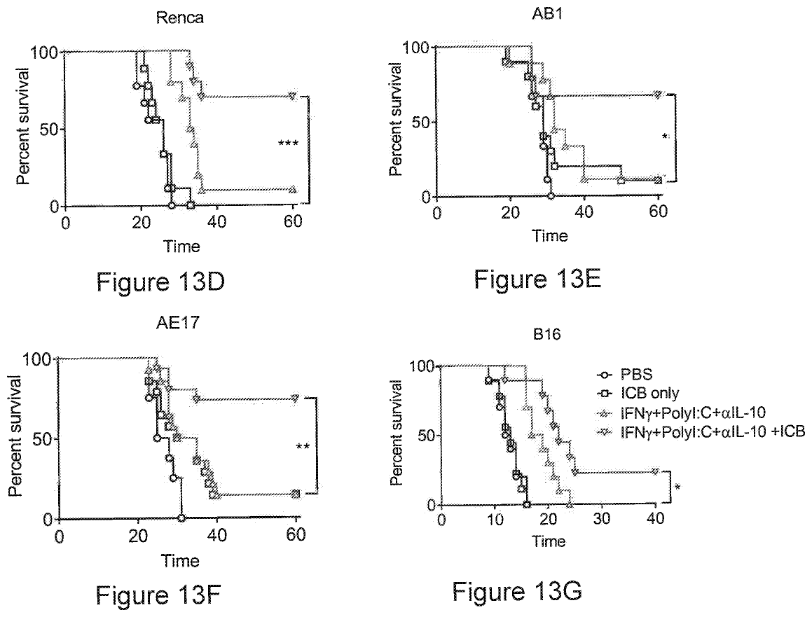

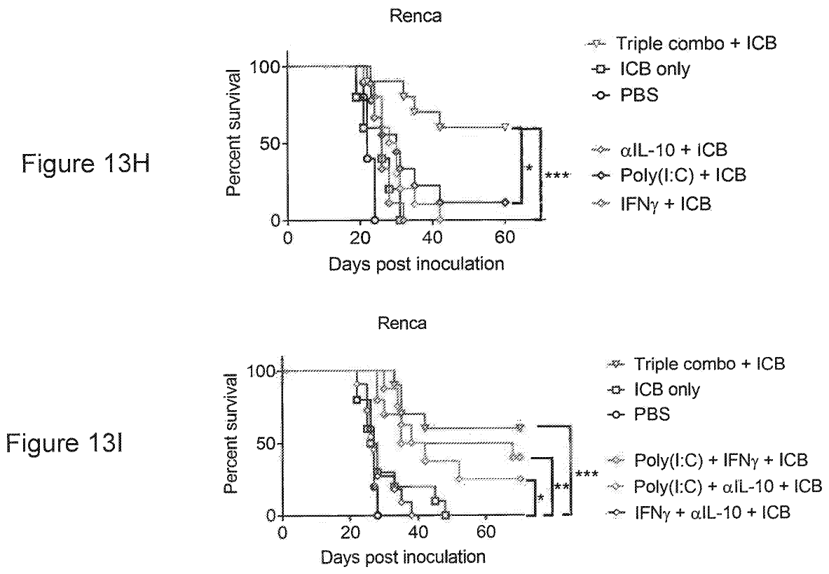

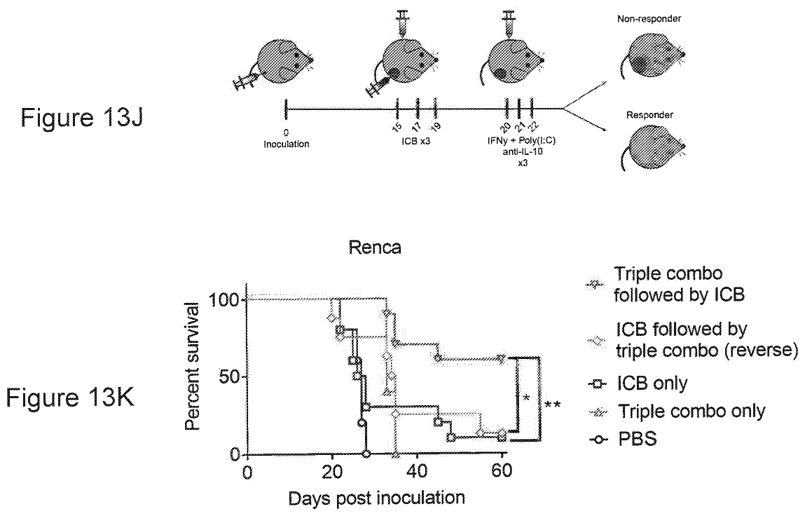

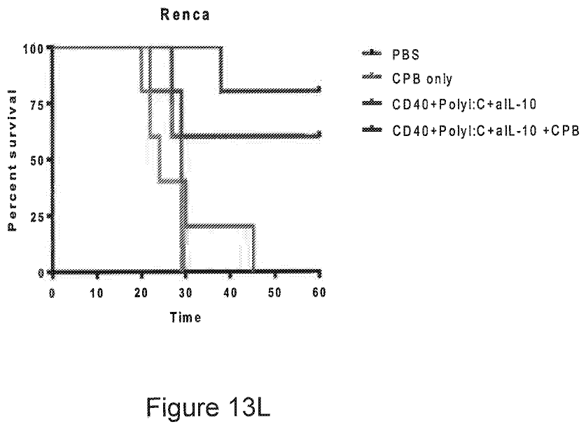

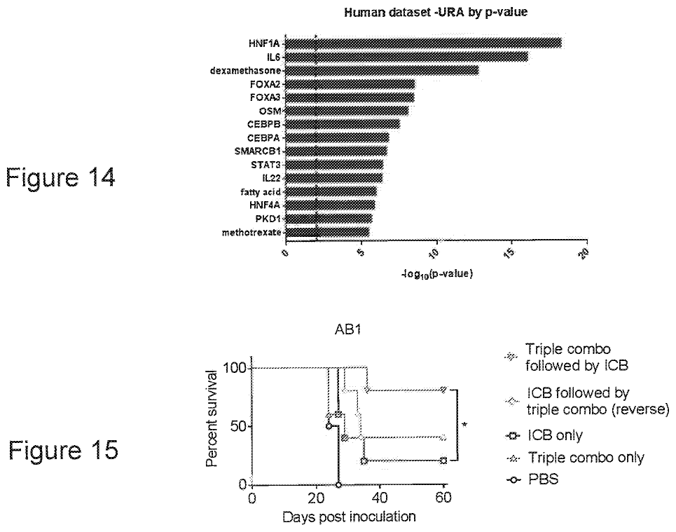

In order to identify higher-level regulators of the inflammatory pathways and networks enriched in responding tumours which could be therapeutically targeted, upstream regulator analysis (URA) was employed substantially as described by Kramer, A., et al., ((2014) Bioinformatics 30, 523-530) to identify higher-level regulators of the inflammatory pathways and networks enriched in responsive tumours. URA identifies transcriptional regulators that, based on prior experimental data, are known to modulate expression levels of differentially expressed genes. Despite the marked difference in the number of differentially expressed genes and cellular infiltrates between the AB1 and Renca models, the regulators associated with response to ICB were highly similar, with the top positive regulators for both AB1 and Renca tumours models were IFNγ and STAT1 and the top negative regulator was identified as IL-10 (see FIG. 13A and FIG. 12). Accordingly, despite the marked difference in the number of differentially expressed genes between the models, the upstream regulators associated with response were very similar. Next, URA was used to identify potential drivers of the gene co-expression module associated with response. Again, IFNγ and STAT1 were identified as the top positive regulators and IL-10 as the top negative regulator (data not shown). Importantly, the patient cohort (Mariathasan, Nature 2018;554(7693):544-548) showed similar results (see FIG. 13B). Other negative regulators were also identified in this patient cohort as shown in FIG. 14.

Having identified a regulator signature associated with response, the inventors reasoned that, by targeting these regulators predicted to phenocopy this signature before ICB, it would be possible to convert non-responsive to responsive tumours. To this end, the inventors focused on therapeutics that have been clinically readily available (at least in phase II clinical trials), and therefore chose a pretreatment schedule of IFNγ, anti-IL10 antibody, and/or the TLR3 agonist Poly-I:C, which was also demonstrated in the results shown in FIG. 13A as one of the top positive regulators and which was known to induce STAT1 (Dempoya et al., (2012) J Virol 86, 12760-12769). To this effect, inventors administered a short course of these treatments, for three days only, followed by ICB therapy two days later (FIG. 13C). In addition to using the AB1 and Renca models as described above, inventors also used AE17 mesothelioma and B16 melanoma-bearing C57BL/6 mice, as both were relatively resistant to ICB (Mosely et al., (2017) Cancer Immunol Res 5, 29-41). For example, in all cases, it was observed that mice pretreated with a IFNγ, anti-IL10 antibody, and Poly-I:C were sensitized to ICB (i.e., to therapy with an ICB agent), with significantly increased response rates, from 0-10% for ICB alone, to up to 80% for the combination of IFNγ, anti-IL10 antibody, and Poly-I:C (FIG. 13D to G).

To determine whether the sensitising outcome was the effect of a single drug or due to the combination of the three sensitising agents, mice were pretreated separately with one of the sensitising agent alone followed by ICB. In each case some sensitisation of the tumour to the ICB was noted using each sensitising agent when used separately (FIG. 13H). In addition, pretreatment with a combination of Poly-I:C and IFNγ or a combination of Poly-I:C and anti-IL10 antibody resulted in sensitization of some tumours with an observed added benefit to ICB therapy alone (i.e., ICB agent when applied without a pre-treatment with a sensitising agent) (FIG. 13I). Furthermore, pretreatment with the combination of all three sensitizing agents i.e., Poly-I:C, IFNγ and anti-IL10 antibody resulted in even better sensitisation of the tumours to ICB therapy i.e., compared to ICB therapy alone (FIG. 13H).

In addition, Renca tumour-bearing mice were pretreated with a combination CD40 agonistic antibody, Poly-I:C and IL-10, followed by ICB (anti-CTLA4/anti-PD-L1) treatment and the results are shown in FIG. 13L. These results also implicate use of agonist of CD40 such as a CD40 antibody as a further agent for promoting or enhancing sensitivity of neoplastic cell populations and/or tumours to ICB.

Next, to confirm further whether use of these sensitising agents was indeed promoting or enhancing sensitivity of tumours to ICB immunotherapy, the inventors used the triple combination of Poly-I:C, IFNγ and anti-IL10 antibody to test for this. To this effect, rather than enforcing the effector response, the changed the scheduling and the response of tumours to ICB was compared when the combination of the three sensitizing agents was added to the tumours following ICB (FIG. 13C) to the results observed when ICB followed treatment with the combination of the three sensitizing agents (FIG. 13J). When checkpoint blockade was given first, followed by the triple combination, response rates were similar to ICB alone. However, when the triple combination was given first, even though start of ICB was substantially delayed compared to the controls, the tumours had been sensitized and responded to therapy, in both Renca (FIG. 13K) and AB1 (FIG. 15) mice tumour models.

Accordingly, using these multiple mouse tumour models (AB1 and AE17 mesothelioma, Renca kidney cancer and B16 melanoma), inventors demonstrated that mice that were pre-treated with the one or more sensitising agents such as those selected from Poly-I:C, IFNγ and anti-IL10 were able to become sensitised to checkpoint blockade therapy These data demonstrate a rational for use of one or more sensitising agents, for example two or more of such agents as therapeutics which promote or enhance sensitization of neoplastic cells and/or tumours to ICB. This clears the way to a two-step approach to treating cancer patients where, based on tumour profiling, a decision to treat with ICB can be made initially or after pre-treatment with sensitizing therapeutics.

### Example 7: Induction and/or Enhancement of IFN Alpha/Beta Signalling such as by Induction/Activation of the IFN Alpha/Beta Receptor (IFNAR) such as by Treatment of Tumours with IFN Alpha also Promotes or Enhances Sensitivity to ICB Therapy

This example demonstrates that composition such as a therapeutic, for example antibodies, which is capable of activating and/r enhancing activity of the any drug that would induce IFN alpha/beta would be capable of inducing, promoting or enhancing sensitivity of one or more neoplastic cell populations such tumours ICB such as by increased NK cell numbers and STAT1 phosphorylation in the tumour cellular microenvironment environment.

Based on the above results obtained demonstrating utility of Poly-I:C and any Poly-LC-based combination of sensitising agents, the inventors hypothesised that the induction, promotion or enhancement of sensitivity of tumours to ICB following treatment with poly(I:C) alone or by poly(I:C)-based combinations was due to the induction of IFN alpha/beta pathway.

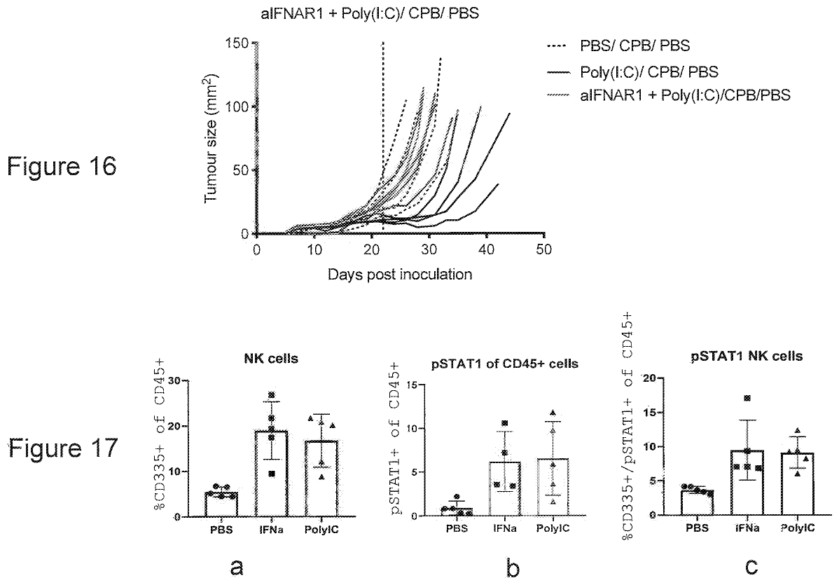

To test this, AE17-bearing mice were pre-treated with poly(I:C) followed by ICB, while at the same time blocking the IFN alpha/beta receptor (IFNAR) by cotreatment with blocking antibodies. Indeed, the beneficial effect of poly(I:C) pretreatment was abrogated by IFNAR blockade (FIG. 16). In other words, following on from the inventors' reasoning that poly(I:C) may work to induce, promote or enhance sensitivity inter alai through the induction of interferon alpha/beta pathway, the inventors tested the anti-tumour effect of pretreatment with poly(I:C) against interferon alpha/beta receptor and observed that this effect was abolished (FIG. 16).

Based on this data, the inventors then reasoned that any compound, drug or composition of matter that capable of activating or enhancing activity of IFN alpha/beta signalling would be capable of inducing, promoting or enhancing the response-associated tumour microenvironment environment, characterised by increased NK cell numbers and STAT1 phosphorylation. Accordingly the inventors next sought to establish whether other therapeutic compounds or compositions that would induce IFN alpha/beta signalling would be capable of promoting or enhancing sensitivity of neoplastic cell populations such as tumours to ICB. To test for this, inventors pretreated AE-17-bearing mice with poly(I:C) or recombinant IFN alpha and tested for their effects on attracting NK cells and activating STAT1 in the tumour microenvironment. Inventors found that IFN alpha indeed induced a highly similar profile in tumours, characterised by increased NK infiltration and STAT1 phosphorylation (i.e., pSTAT1 activation) (FIG. 17).

### Example 8: Sensitizing Treatment Induces a Responsive Phenotype Characterized by Increase in NK Cells (Enhanced NK Infiltration) and Activation/Phosphorylation of STAT1 in the Cellular Microenvironment of Tumours

This example demonstrates that sensitising increased phosphorylation of STAT1 (i.e., STAT1 activation) and also increased NK cell population in the tumour cellular microenvironment.

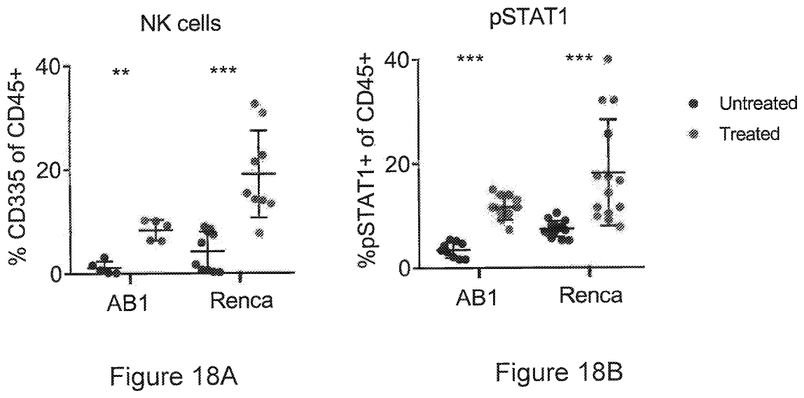

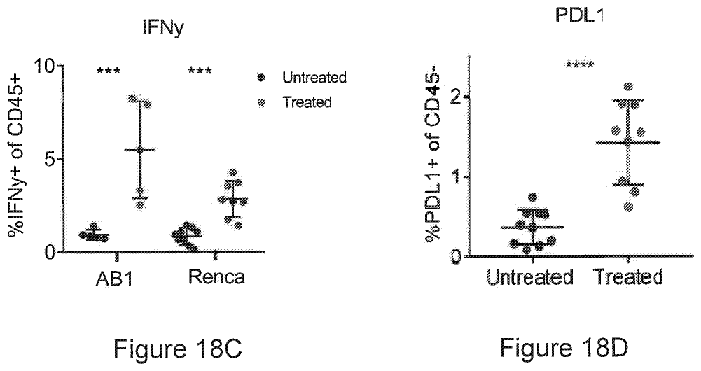

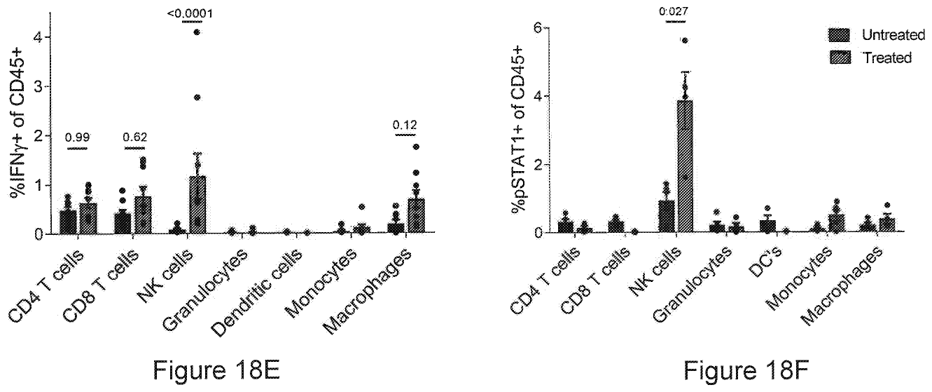

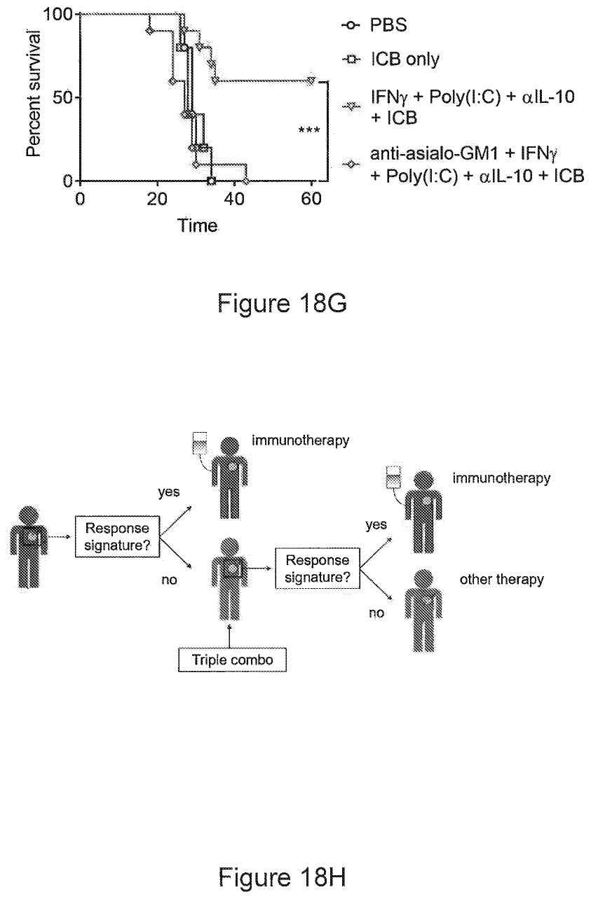

To test whether the sensitizing therapeutic agents such as the combination of sensitising agents described in the proceeding examples, induced response-associated phenotype of promoting or enhancing sensitivity of of the tumours to ICB, the tumours were tested for STAT1 activation and NK infiltration after 3 days of pretreatment of mice bearing tumours with a combination of IFNγ, poly(I:C) and anti-IL10, or vehicle controls (FIG. 18A). Indeed, it was found that the sensitizing pretreatment markedly increased the frequencies of CD335⁺ cells (FIG. 18A and FIG. 19A). In addition, there was an increase in phospho-STAT1 positive and IFNγ-producing leukocytes infiltrating the tumours (FIGS. 18B, C and D). In addition, the pretreatment increased PD-L1 and pSTAT1 positive tumour cells (FIG. 18D and FIG. 19B). Although the pre-treatment-resulted in an increase in IFNγ production which was derived from multiple leukocyte subsets in the tumour, this was significant for the NK cell population only (p<0.001) but not for the other cell subsets (FIG. 18E). This was also the case for phosphorylation of STAT1 (FIG. 18F). Further phenotypic characterization of these CD335⁺ NK cells in the tumours revealed that they were conventional NK cells, and not tissue resident CD335⁺ ILC1 or ILC3 (FIG. 20) (Vivier et al., (2018) Cell 174, 1054-1066). High expression of activation markers CD11b and KLRG1 confirmed the activated and terminally differentiated state of these conventional NK cells (data not shown). When NK cells were depleted prior to pretreatment, the sensitizing effect to ICB was completely abolished (FIG. 18G), demonstrating that indeed this was mediated through treatment-induced infiltration of circulating NK cells in the tumour microenvironment.

Taken together, the data presented herein demonstrates that a pretreatment tumour microenvironment dominated by infiltrating NK cells and an inflammatory gene expression signature characterized by STAT1 activation which is sensitive to ICB and that this profile can be therapeutically exploited. These data clear the way to a two-step approach to treating cancer patients, in which tumour profiling allows a decision to treat with ICB either initially (e.g., if based on profiling the tumours are shown to be sensitive to ICB) or, alternatively, after pretreatment with one or more sensitizing therapeutics/agents (e.g., if tumour profiling shown the tumours to be resistant or partially sensitive to ICB) (FIG. 18H).

### Example 9: Retinoids Sensitize Tumour Cellular Microenvironment to ICB

This example demonstrates that retinoids (such as all-trans retinoic acid, bexarotene and/or isotretinoin) are able to sensitize tumour cellular microenvironment to ICB.

Since tretinoin (also known as all-trans retinoic acid) was identified as one of the top predicted upstream regulators of the response-associated gene expression signature with a p value of 0.000954 and a z-score of 2.565, the inventors sought to test the ability of retinoids in general, and as only example tretinoin more specifically, to sensitize the tumour cellular microenvironment to checkpoint blockade (ICB).

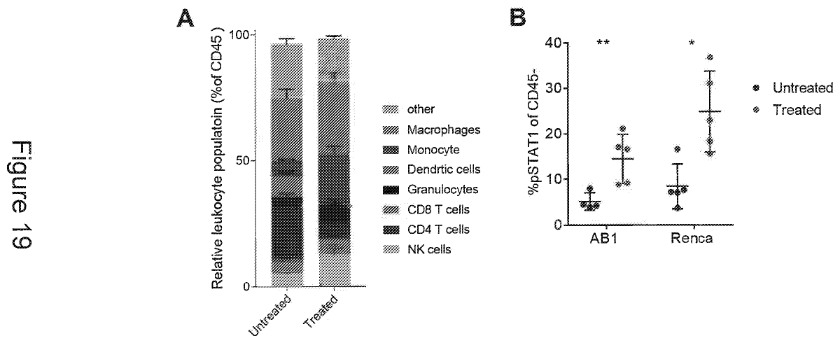

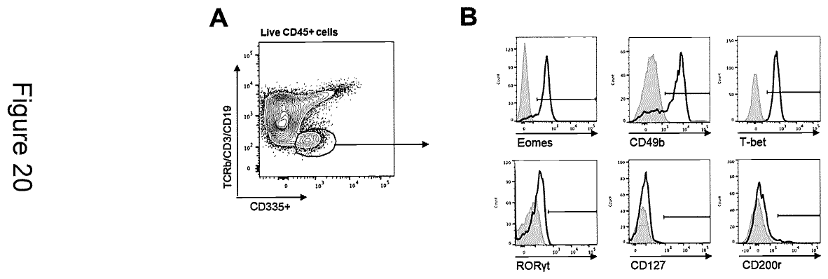

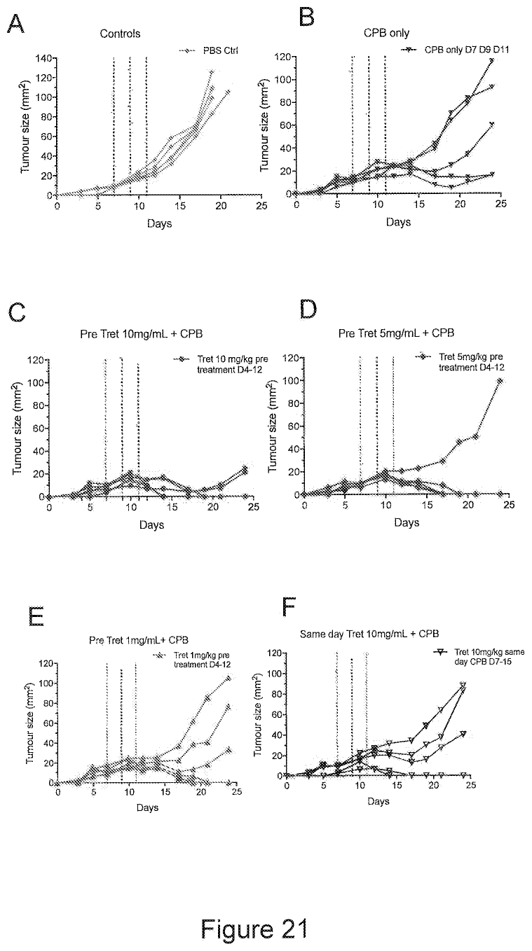

As shown in FIG. 21 mice with AB1-HA mesothelioma tumours were treated with checkpoint blockade (ICB) antibodies (anti-CTLA4 antibody and anti-PD-L1 antibody) with or without tretinoin. The tretinoin treatment started either 3 days before CPB (‘Pre Tret’)) or at the same time as the CPB (‘Same day Tret’). Tretinoin was dosed at three dosages; 10 mg/kg, 5 mg/kg or 1 mg/kg, as indicated. CPB antibodies were given day 7-9-11 after tumour inoculation, tretinoin was given daily for 9 days in total (grey shaded area) and started either 3 days (day 4) before or concomitantly with CPB (starting day 7). The results shown in FIG. 21 demonstrate that treatment of mice bearing cancers with a retinoid such as for example tretinoin prior to ICB, improved the response of the tumours to ICB in a dose-dependent manner. Furthermore, this data demonstrates that administering the retinoid, such as tretinoin, to mice before ICB markedly improved the efficacy of ICB compared to when the retinoid was administered at same time as ICB.

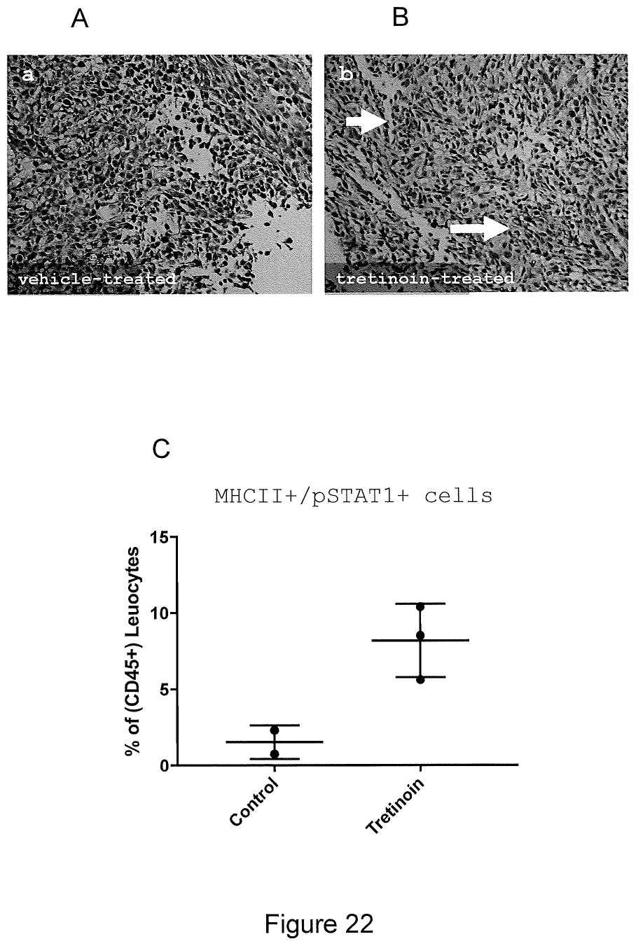

In another experiment, AB1-bearing mice were treated with tretinoin 10 mg/kg for 5 days, and tumours were harvested for immunohistochemistry. As shown in FIG. 22 (panels A and B), a retinoid such as tretinoin induced the checkpoint blockade response-associated phenotype with Immunohistochemistry characterized by increased patches of pSTAT1 immune cell infiltrates in tumours after treatment with a retinoid such as tretinoin in CT26 colorectal tumours. As show in FIG. 22, areas of pSTAT1 positive infiltrates in the tretinoin-treated group, but not in vehicle controls. These findings were corroborated in Renca kidney cancer using flow cytometry, showing increased pSTAT1 positive leukocytes in tumours, particularly in MHC class II positive cells (FIG. 22A).

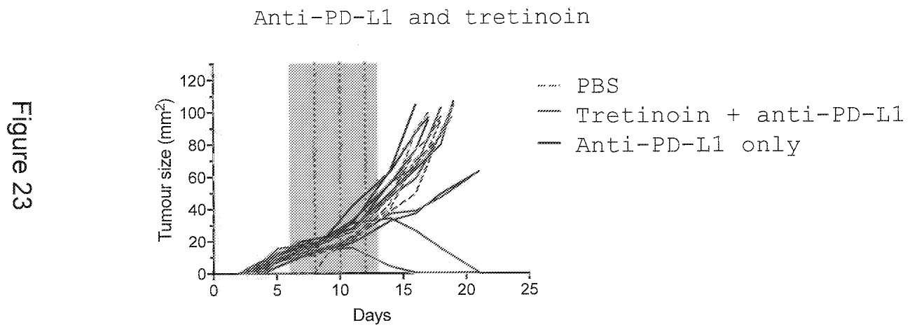

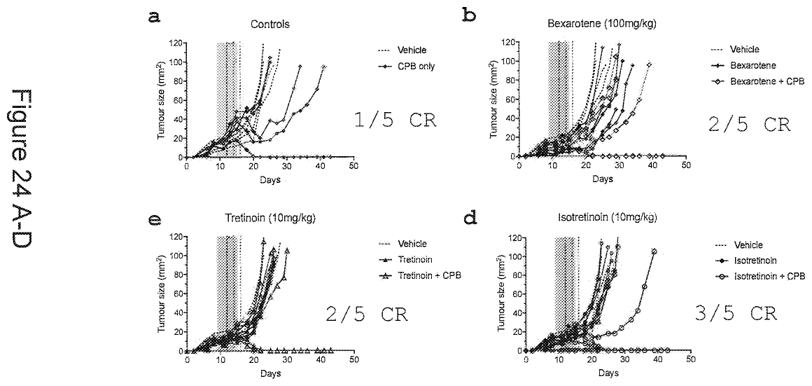

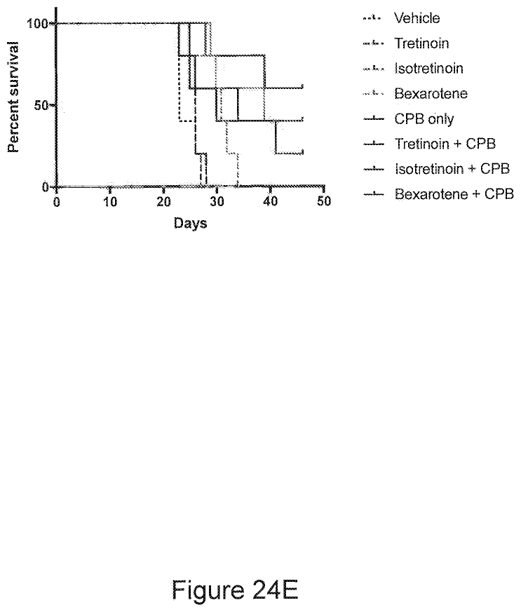

The sensitizing beneficial effects of retinoids were not limited to use of ICB therapy with a combination of the two ICB antibodies against anti-CTLA4 and anti-PD-L1. This is because treatment with anti-PD-L1 antibody alone (which blocks the PD-1/PD-L1 axis) as ICB (i.e., in absence of anti-CTLA4 antibody) also resulted in increased efficacy (FIG. 23). Similarly, the sensitizing beneficial effects would apply to the broader class of retinoids compounds and were not limited to tretinoin alone. This is because the sensitizing effect was achieved also with other retinoids, including bexarotene and isotretinoin (FIG. 24), which showed an increased percentage of complete regression after combination therapy with ICB, compared to ICB alone.

In summary, the results of this example demonstrate that retinoids such as tretinoin, bexarotene and isotretinoin are able to improve the tumour response to ICB in a dose-dependent manner, and are most efficacious when the retinoids are administered to subjects prior to ICB and less efficacious when administered at the same time as ICB (see e.g., FIG. 22).

Retenoids, for example tretinoin, bexarotene and isotretinoin, are also able to induce the checkpoint blockade response-associated phenotype characterised in CT26 colorectal tumours by increased pSTAT1 immune cell infiltrates in tumours after administration of the retinoids (FIGS. 22A and 22B). These findings were corroborated in Renca kidney cancer using flow cytometry, showing increased pSTAT1 positive leukocytes in tumours, particularly in MHC class II positive cells (FIG. 22C).

The beneficial effects of retinoids, for example tretinoin, bexarotene and isotretinoin, were not limited to the type of ICB immunotherapy (FIG. 23). Similarly, the beneficial effects of retinoids in sensitising tumours to ICB were not limited to tretinoin alone, but could be extended to other drugs from the retinoid class, including bexarotene and isotretinoin (FIG. 24). The results outlined in this example, support inclusion of retinoids such as all trans retinoic acid/tretinoin, bexarotene and/or isotretinoin, as sensitising agents for promoting or enhancing sensitivity of one or more neoplastic cells to immune check point blockade. The results outlined herein provide further support to the finding outlined in the proceeding examples demonstrating that a pre-treatment tumour microenvironment dominated inflammatory gene expression signature driven by STAT1 sensitizes to ICB and that this profile can be therapeutically exploited, for example in the two-step approach to treating cancer patients outlined earlier above (where based on tumour profiling a decision to treat with ICB can be made initially or after pre-treatment with one or more sensitizing agents).

1. A method for promoting or enhancing the sensitivity of one or more neoplastic cells and/or neoplastic tumours to immune checkpoint blockade agents, said method comprising the step of: administering to a neoplastic cell and/or a neoplastic tumour, prior to treatment of immune checkpoint blockade agents, one or more immune checkpoint sensitising agents or exposing the cell and/or tumour to the one or more sensitising agents to thereby cause the cells and/or tumour to become sensitized to an immune checkpoint blockade agent.

2. A method for promoting or enhancing the sensitivity of one or more neoplastic cells and/or neoplastic tumours to immune checkpoint blockade agents, said method comprising the step of:
a) administering to a neoplastic cell and/or a neoplastic tumour, prior to treatment of immune checkpoint blockade agents, one or more immune checkpoint sensitising agents, or exposing the cell and/or tumour to the one or more sensitising agents, for a period of time and/or at a therapeutic amount that causes a tumour to become sensitized to an immune checkpoint blockade agent.

3. A method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase the numbers of NK cells and thereby promote or enhance the sensitivity of the neoplastic cell and/or tumour to an immune check point blockage agent.

4. A method for promoting or enhancing the sensitivity of one or more neoplastic cells and/or neoplastic tumours to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by the cell and/or tumour thereby promoting or enhancing the sensitivity of the one or more neoplastic cells an immune check point blockage agent.

5. A method for promoting or enhancing the sensitivity of a neoplastic cell and/or neoplastic tumour to immune checkpoint blockade agents, said method comprising the step of administering to a neoplastic cell and/or a neoplastic tumour, prior to treatment of immune checkpoint blockade agents one or more immune checkpoint sensitising agents, or exposing the cell and/or tumour to the one or more sensitising agents, to increase production of IFNγ and/or activated STAT1 protein by NK cells thereby promoting or enhancing the sensitivity of the neoplastic cell and/or tumour to an immune check point blockage agent.

6. A method for promoting or enhancing the sensitivity of a neoplastic cell and/or tumour to immune checkpoint blockade agents, said method comprising the step of:
a) identifying a neoplastic cell and/or tumour that is resistant to one or more immune checkpoint blockade agents; and
b) administering or exposing the neoplastic cell and/or tumour identified in step (a) to a therapeutically effective amount of one or more immune checkpoint sensitising agents, for at least 3 days prior to immunotherapy or until the tumour is at least partially sensitized to an immune checkpoint blockade agent.

7. A method according to any one of claims 1 to 6 wherein the immune checkpoint sensitising agents are selected from: a CD40 agonist, an anti-IL10 antibody, an inducer of interferon alpha/beta signalling, an interferon gamma or a functional variant thereof and/or a retinoid.

8. A method according to any one of claims 1 to 6 wherein a combination of immune checkpoint sensitising agents are used in the method, said combination being at least a plurality of the identified sensitising agents selected from the group comprising: a CD40 agonist, an anti-IL10 antibody, an inducer of interferon alpha/beta signalling, and an interferon gamma or a functional variant thereof.

9. A method according to claims 8 wherein the immune checkpoint sensitising agents comprise at least a retinoid.

10. A method according to claims 8 wherein the immune checkpoint sensitising agents comprise at least a retinoid and any one or more of a CD40 agonist and/or an anti-IL10 antibody and/or an inducer of interferon alpha/beta signalling and/or an interferon gamma or a functional variant thereof.

11. A method according to claims 8 wherein the immune checkpoint sensitising agents comprise at least an inducer of interferon alpha/beta signalling such as Poly(I:C).

12. A method according to claims 8 wherein the immune checkpoint sensitising agents comprise an inducer of interferon alpha/beta signalling such as Poly(I:C), an anti-IL10 antibody and interferon gamma or a functional variant thereof.

13. A method according to claims 8 wherein the immune checkpoint sensitising agents comprise at least an inducer of interferon alpha/beta signalling such as Poly(I:C), and any one or more of: anti-IL10 antibody and/or interferon gamma or a functional variant thereof and/or a CD40 agonist such as agonistic CD40 antibody.

14. A method according to claims 8 wherein the immune checkpoint sensitising agents comprise at least an inducer of interferon alpha/beta signalling such as Poly(I:C), and any one or both of an anti-IL10 antibody and/or interferon gamma or a functional variant thereof.

15. A method according to claims 8 wherein the immune checkpoint sensitising agents comprise at least a CD40 agonist such as an agonistic anti-CD40 antibody.

16. A method according to anyone of the preceding claims wherein the neoplastic cell population in step (a) of this method is selected by either (i) exposing the cells to one or more immune checkpoint blockade agents and identifying those cells that are resistant to the immune checkpoint blockade agents or (ii) by measuring the activity of STAT 1 in a cell population wherein by the absence of activation of the STAT1 protein (less than 50% of cells positive for nuclear STAT1 or phosphorylated STAT1) presents as a biomarker for resistance of the cell population of step (a) to immune checkpoint blockade agents.

17. A method according to anyone of the preceding claims wherein the cells of step (b) are exposed to an immune checkpoint blockade agent after measurable amounts of (i) the STAT1 biomarker are detected in the cell population and/or (ii) measurable amount of natural killer cells are detected in the tumour cellular microenvironment.

18. A method according to anyone of the preceding claims wherein the method includes the step of administering an immunotherapy or an immune checkpoint blockade agent once the one or more checkpoint sensitising agents have resulted in sufficient increase in the amount of the immune effectors IFNγ and/or activated STAT1 at the tumour or neoplastic cell population environment for enhancing the efficacy of the immune therapy or immune checkpoint blockade agent(s) on a malignant condition.

19. Use of one or more sensitising therapeutic agents selected from an agonistic CD40 antibody, anti-IL10, TLR3 ligand Poly(I:C), a retinoid (such as all-trans retinoic acid) and/or Interferon gamma, in the manufacture of a medicament for treating a tumour wherein said tumour is resistant to an immune checkpoint blockade agent.

20. Use of a therapeutically effective amount of one or more immune checkpoint sensitising agents, in the manufacture of a medicament for sensitising a tumour wherein said tumour is resistant to an immune checkpoint blockade agent, wherein said medicament increases the numbers of NK cells (such as NK cells producing activated STAT1- and/or IFNγ) and/or increases IFNγ and/or activated STAT1 production by neoplastic cells and/or tumour cells and/or NK cells.

21. A use according to claim 19 or 20 wherein the medicament includes instructions to administered to a tumour that is resistant to one or more immune checkpoint blockade agents, the immune checkpoint sensitising agents at least 3 days prior to an immunotherapy.

22. A method for treating a patient with a malignant condition or a patient that is predicted to develop a malignant condition, said method comprising the step of:
a) identifying a tumour or neoplastic cell population(s) that is resistant to one or more immune checkpoint blockade agents; and
b) administering or exposing the tumour or neoplastic cell population(s) identified in step (a) to a therapeutically effective amount of one or more immune checkpoint sensitising agents, for at least 3 days prior to immunotherapy until the tumour is at least partially sensitized to an immune checkpoint blockade agent.

23. The method according to claim 22 wherein the neoplastic cell population in step (a) of this method is selected by either (i) exposing the cells to one or more immune checkpoint blockade agents and identifying those cells that are resistant to the immune checkpoint blockade agents or (ii) by measuring the activity of STAT 1 in a cell population wherein the absence of activation of the STAT1 protein (less than 50% of cells positive for nuclear STAT1 or phosphorylated STAT1) presents as a biomarker for resistance of that cell population in step (a) to immune checkpoint blockade agents.

24. The method according to claim 22 wherein the cells of step (b) are exposed to an immune checkpoint blockade agent after measurable amounts of (i) the STAT1 biomarker are detected in the cell population and/or (ii) measurable amount of natural killer cells are detected in the tumour cellular microenvironment.

25. Use of one or more sensitising therapeutic agents selected from an agonistic CD40 antibody, anti-IL10, TLR3 ligand Poly(I:C), a retinoid (such as all-trans retinoic acid) and/or Interferon gamma, for promoting or enhancing, in a patient, the sensitivity of a tumour to immune checkpoint blockade agents wherein the therapeutic agent is administered to the tumour that is resistant to one or more immune checkpoint blockade agents at least 3 days prior to immunotherapy.

26. Use of one or more therapeutic agents selected from an agonistic CD40 antibody, anti-IL10, TLR3 ligand Poly(I:C), a retinoid (such as all-trans retinoic acid) and/or Interferon gamma, in the manufacture of a medicament for treating a tumour in a patient, wherein said tumour is resistant to an immune checkpoint blockade agent.

27. A therapeutic composition for use in sensitizing a tumour to immune checkpoint blockade agents comprising: one or more of an agonistic CD40 antibody, anti-IL10, TLR3 ligand Poly(I:C), a retinoid such as all-trans retinoic acid and/or Interferon gamma, and a pharmaceutically acceptable carrier.

28. A therapeutic according to claim 27 wherein comprising a therapeutically effective amount of a combination of at least two therapeutics selected from: anti-IL10, Poly(I:C) and interferon gamma or anti-CD40, anti-IL10, retinoids such as all-trans retinoic acid and interferon gamma.

29. A therapeutic according to claim 27 wherein comprising a therapeutically effective amount of a combination of at least three therapeutics selected from: anti-IL10, Poly(I:C) and interferon gamma or anti-CD40, anti-IL10, retinoids such as all-trans retinoic acid and interferon gamma.

30. A therapeutic for use in sensitizing a tumour to immune checkpoint blockade agents comprising a therapeutically effective amount of a combination of anti-IL10, Poly(I:C) and interferon gamma or anti-CD40, anti-IL10 and interferon gamma.

31. A kit for treating a tumour or a population of neoplastic cells the kit comprising:
a) a therapeutically effective amount of one or more immune checkpoint sensitising agents, and
b) instructions to administer the immune checkpoint sensitising agents to a tumour that is resistant to one or more immune checkpoint blockade agents at least 3 days prior to an immunotherapy.

32. A kit according to claim 31 wherein the kit includes one or more immune checkpoint blockade agents and/or immunotherapeutic agents, and instructions to administer the agent or agents to the tumour or neoplastic cell population once the one or more checkpoint sensitising agents have attracted sufficient effector immune cells (e.g. IFNγ and/or activated STAT1 producing NK cells) to the tumour or neoplastic cell population environment.

33. A diagnostic or method for predicting a response to immune checkpoint blockade comprising the steps of:
a. measuring STAT1 activation in a cell population; and
b. determining whether the cell population is resistant to immune checkpoint blockade agents wherein the activation and/or localisation of the biomarker STAT1 is indicative of the cells sensitivity to one or more immune checkpoint blockade agents.

34. A diagnostic or method for predicting a response to immune checkpoint blockade comprising the steps of:
a. measuring natural killer cell presence in a tumour; and
b. determining whether the tumour is resistant to immune checkpoint blockade agents wherein the activation and/or presence of natural killer cells is indicative of the cells sensitivity to one or more immune checkpoint blockade agents.

35. A method for immobilising NK cells or increasing the number of NK cells at site of a neoplastic cell and/or tumour in a subject and/or to the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

36. A method of inducing or increasing production of IFNγ and/or activated STAT1 protein by NK cells in a subject, for example at a site of the neoplastic cell and/or tumour in the subject and/or in the cellular microenvironment of the neoplastic cell and/or tumour in the subject, said method comprising administering to the subject one or more sensitising agents selected from a CD40 agonist, an anti-IL10 antibody, prior to treatment of the subject with one or more immune check point blockade agents.

37. A method of inducing or increasing production of IFNγ and/or activated STAT1 protein by a neoplastic cell and/or tumour in a subject, said method comprising administering to the subject one or more sensitising agents selected from a CD40 agonist, an anti-IL10 antibody, prior to treatment of the subject with one or more immune check point blockade agents.
